(19) United States
(12) Patent Application Publication
(10) Pub. No.: US 2023/0136331 A1
(43) Pub. Date: May 4, 2023

(54) IMMUNOCYTOKINE CONTAINING IL-21R MUTEIN

(71) Applicant: ILDONG PHARMACEUTICAL CO., LTD., Seoul (KR)

(72) Inventors: Hong Seok BAN, Hwaseong-si (KR); Seok-Woo YANG, Hwaseong-si (KR); Hye Mi KWON, Hwaseong-si (KR); Jea Won CHO, Hwaseong-si (KR); Suh-Youn SHON, Hwaseong-si (KR); Do Sup LEE, Hwaseong-si (KR); Ji-Hyung LEE, Hwaseong-si (KR); Seong Wook LEE, Hwaseong-si (KR); Ukjin SON, Hwaseong-si (KR); Jun Su BAN, Hwaseong-si (KR); Jihye KIM, Hwaseong-si (KR)

(21) Appl. No.: 17/937,428

(22) Filed: Sep. 30, 2022

Related U.S. Application Data
(60) Provisional application No. 63/250,911, filed on Sep. 30, 2021.
(60) Provisional application No. 63/351,298, filed on Jun. 10, 2022.

(51) Int. Cl. C07K 16/18; A61P 35/00

(52) U.S. Cl. CPC C07K 16/18; A61P 35/00; C07K 2317/52; C07K 2317/565

(57) ABSTRACT

Provided herein is an immunocytokine comprising (i) an antigen binding protein (ABP) specific to a target protein; (ii) an IL-21 domain; and (iii) an IL-21Rα mutein, wherein the IL-21Rα mutein has a reduced binding affinity to the IL-21 domain compared to a wild-type IL-21Rα. Further provided includes a method of using the immunocytokine to selectively activate an IL-21Rα on a target cell, thereby enhance immune response or treat cancer.

### 1. CROSS REFERENCE TO RELATED APPLICATIONS

This application claims priority to and the benefit of U.S. Provisional Patent Application Nos. 63/250,911 filed on Sep. 30, 2021 and 63/351,298 filed on Jun. 10, 2022, the entire contents of which are incorporated by reference herein.

### 2. SEQUENCE LISTING

The instant application contains a sequence listing with 240 sequences which has been submitted via USPTO Patent Center is hereby incorporated by reference in its entirety. Said XML copy, created Sep. 29, 2022, is named “50042US_015US_CRF_sequencelisting.xml” and is 301,157 bytes in size.

### 3. BACKGROUND OF THE INVENTION

For the last decade, immune checkpoint blockade (ICB) represented by anti-PD-1 or anti-CTLA-4 antibody has led to considerable success in cancer immunotherapy, in which ICBs reprogram the immune system of patients to be against cancer. Despite the outstanding effectiveness of these types of therapeutics, few patients have benefitted from ICBs because the most patients failed to develop durable immune responses and stop the progression of cancer growth. The long-lasting and durable effector function of activated T cells is essential for eliminating cancer cells from our body through T cell-mediated immune response. In chronic infection and cancer, most of the T cells exposed to persistent antigens followed by continuous T cell receptor stimulation are exhausted. The exhausted T cells in the tumor microenvironment show dysfunction of cytokine releases like IFN-γ and TNF-α, which is their major effector function and loss of proliferation capacity. Exhausted T cells are distinguished from effector and memory T cells by high level expression of co-inhibitory receptors such as PD-1, TIM-3, or CTLA-4 on their surface. Another noticeable feature of fully differentiated exhausted T cells is epigenetic stability which might be the main reason for the resistance to ICB treatment.

A recent study done by Kristen E. Pauken reported that the epigenetic fate inflexibility of the genome of exhausted T cells impedes the transition of exhausted T cell into memory T cell, which is expected to be triggered by ICB treatment. This suggests that epigenetic reprogramming of exhausted T cells into memory T cells which have the potential for self-renewal and durable effector function, might be a solution for the limitation of current cancer immunotherapeutic.

Epigenetic reprogramming is accompanied by changes in the expression level of writer enzymes such as histone methyl transferases (HMT), histone acetyl transferases (HAT), or DNA methyltransferase (DNMT), all of which can alter the chromatin states determining the expression or suppression of a gene. It is well known that the signal triggered by a cytokine in immune cells regulates the expression level or activity of writer enzymes, which determines the differentiation fate of immune cells. From all types of cytokines, gamma chain cytokines, namely IL-2, IL-4, IL-7, IL-9, and IL-21, are known that have prominent roles in the activation of effector T cells or differentiation of memory T cells, suggesting that they can be potential candidates for anti-cancer immunotherapeutic. These cytokines can cause changes in chromosome accessibility and chromatin structure by altering the expression level of several transcription factors responsible for epigenetic modification. For example, TCF-1, a transcription factor expressed in T cells, is known that has intrinsic HDAC (histone deacetylase) activity and regulates gene expression by modifying chromatin accessibility. It was reported that the expression of TCF-1 in T cells can be induced by the treatment of cytokines like IL-7, IL-15, or IL-21 in vitro culture or in vivo experiment. Recently, lineage tracing based on single-cell sequencing analysis elucidated that TCF-1 is a key biomarker for progenitor exhausted CD4+ or CD8+ T cells (TPEX) respond to ICB treatment. This means that manipulating the expression of transcription factors like TCF-1 induced by cytokine in T cells can be another option for cancer immunotherapy. For several decades, there have been attempts to use these cytokines for cancer immunotherapy.

However, the clinical utility is minimal because of severe dose-limiting toxicities, leading a patient to death. In general, the expression of a cytokine receptor is ubiquitous all over the body, and the treatment of high doses of cytokine is related to systemic toxicities. Therefore, enhancing the specificity of a cytokine to increase the tolerable dose for systemic administration is required to solve toxicity-relating problems.

### 4. SUMMARY OF THE INVENTION

The present disclosure provides a novel immunocytokine specific to a target cell. The immunocytokine has activity specific to target cells by comprising a cytokine molecule (IL-21) fused to antigen binding protein (ABP) specific to a target protein and a capping moiety, interfering nonspecific binding of the cytokine molecule to a non-target cell. As a capping moiety, the present disclosure provides IL-21Rα mutein that has a reduced binding affinity to the IL-21 domain compared to a wild-type IL-21Rα.

This immunocytokine binds to a target protein expressed on the surface of a certain cell type (e.g., immune cells) through its ABP, which results in accumulation of a cytokine close to the target cell. If a cytokine of the immunocytokine randomly binds to non-target cells before reaching to its target cell, a high dose of the cytokine might induce various side effects, and it may cause a narrow therapeutic index of the immunocytokine. To avoid this problem, the extracellular domain of IL-21Rα is used as a capping moiety to interfere with the binding of IL-21 to endogenous IL-21Rα (e.g., wild type IL21Rα (IL21RαWT)) on non-target cells. Since non-target cells lack a target protein that the ABP can recognize, the immunocytokine is not targeted to non-target cells and IL-21 stays capped by the capping moiety. Once immunocytokine with the capped IL-21 is delivered to a target cell, the capping moiety, the extracellular domain of IL-21Rα, is stripped off by competition with the endogenous IL21Rα (e.g., IL21RαWT) of a target cell, which can make IL-21 bind to the endogenous IL21Rα and transduce a signal to the target cell.

Since high binding affinity of IL-21 (approximately K_{D}=50 pmol) to the extracellular domain of IL-21Rα can interfere with the competition between the extracellular domain of IL-21Rα of the immunocytokine and endogenous IL-21Rα of target cells, an extracellular domain of IL-21Rα in the immunocytokine was mutated (IL-21RαMutein) to have a lower binding affinity to IL-21. ABP of the immunocytokine can guide the complex comprising IL-21 and IL21RαMutein to specific target cells and the IL-21 brought to the target cells can bind and transduce signal to the target cells by competition between IL-21Rα mutein of the immunocytokine and endogenous IL-21 receptors on the surface of target cells.

Accordingly, the present disclosure provides: an immunocytokine, comprising:
a. an antigen binding protein (ABP) specific to a target protein;
b. an IL-21 domain; and
c. an IL-21Rα mutein,
   wherein the IL-21Rα mutein has a reduced binding affinity to the IL-21 domain compared to a wild-type IL-21Rα.

In some embodiments, the target protein is an immune checkpoint molecule. In some embodiments, the target protein is PD-1, PD-L1, TIGIT, LAG-3, CTLA-4, TIM-3, CD39, CD38, CD73, CD36, CD25, CD47, CD24, CD20, SIPRα, CD40, or CD20.

In some embodiments, the ABP is an antibody against the target protein. In some embodiments, the ABP is an immune check point inhibitor. In some embodiments, the ABP is anti-PD-1 antibody. In some embodiments, the anti-PD-1 antibody is IgG.

In some embodiments, the ABP comprises Fc fragment selected from a human IgG1 Fc fragment, a human IgG2 Fc fragment, a human IgG3 Fc fragment, and a human IgG4 Fc fragment. In some embodiments, the Fc fragment is a human IgG4 Fc fragment. In some embodiments, the Fc fragment comprises the sequence selected from SEQ ID NOs: 16, 185-190.

In some embodiments, the ABP comprises an Fc fragment with two Fc moieties. In some embodiments, the IL-21Rα mutein is linked to the first of the two Fc moieties, and the IL-21 domain is linked to the second of the two Fc moieties. In some embodiments, the IL-21 domain and the IL-21Rα mutein are respectively linked through a non-cleavable peptide linker or without a peptide linker. In some embodiments, the non-cleavable peptide linker is G4S linker having the sequence of SEQ ID NO: 17. In some embodiments, the non-cleavable peptide linker has a sequence selected from SEQ ID NOs: 212-224.

In some embodiments, the ABP is selected from nivolumab, pembrolizumab, cemiplimab, atezolizumab, dostarlimab, durvalumab, avelumab, ipilimumab, tremelimumab, tiragolumab, relatlimab, or a functional variant thereof. In some embodiments, the ABP comprises V_{H} CDR1, V_{H} CDR2, V_{H} CDR3, V_{L} CDR1, V_{L} CDR2, and V_{L} CDR3 sequences of nivolumab, pembrolizumab, cemiplimab, atezolizumab, dostarlimab, durvalumab, avelumab, ipilimumab, tiragolumab, relatlimab, or tremelimumab. In some embodiments, the ABP comprises heavy chain and/or light chain of nivolumab, pembrolizumab, cemiplimab, atezolizumab, dostarlimab, durvalumab, avelumab, ipilimumab, tiragolumab, relatlimab, or tremelimumab. In some embodiments, the ABP comprises a heavy chain variable domain and/or a light chain variable domain of nivolumab, pembrolizumab, cemiplimab, atezolizumab, dostarlimab, durvalumab, avelumab, ipilimumab, tiragolumab, relatlimab, or tremelimumab. In some embodiments, the heavy chain variable domain and/or the light chain domain are linked to a human IgG1 Fc fragment, a human IgG2 Fc fragment, a human IgG3 Fc fragment, or a human IgG4 Fc fragment. In some embodiments, the Fc fragment includes a mutation for knob-in-hole interaction,

In some embodiments, the ABP comprises:
a. a heavy chain having the sequence of SEQ ID NO: 1 and a light chain having the sequence of SEQ ID NO: 2;
b. a heavy chain having the sequence of SEQ ID NO: 3 and a light chain having the sequence of SEQ ID NO: 4;
c. a heavy chain having the sequence of SEQ ID NO: 5 and a light chain having the sequence of SEQ ID NO: 6;
d. a heavy chain having the sequence of SEQ ID NO: 7 and a light chain having the sequence of SEQ ID NO: 8;
e. a heavy chain having the sequence of SEQ ID NO: 9 and a light chain having the sequence of SEQ ID NO: 10;
f. a heavy chain having the sequence of SEQ ID NO: 11 and a light chain having the sequence of SEQ ID NO: 12;
g. a heavy chain having the sequence of SEQ ID NO: 13 and a light chain having the sequence of SEQ ID NO: 14;
h. a heavy chain having the sequence of SEQ ID NO: 151 and a light chain having the sequence of SEQ ID NO: 152;
i. a heavy chain having the sequence of SEQ ID NO: 153 and a light chain having the sequence of SEQ ID NO: 154;
j. a heavy chain having the sequence of SEQ ID NO: 225 and a light chain having the sequence of SEQ ID NO: 226; or
k. a heavy chain having the sequence of SEQ ID NO: 227 and a light chain having the sequence of SEQ ID NO: 228.

In some embodiments, the ABP comprises:
a. a heavy chain having the sequence of SEQ ID NO: 1 or a variation thereof, and a light chain having the sequence of SEQ ID NO: 2, wherein the variation comprises a knob-and-hole mutation and/or removal of Lys (K) at the C-terminal end in SEQ ID NO: 1;
b. a heavy chain having the sequence of SEQ ID NO: 3 or a variant thereof, and a light chain having the sequence of SEQ ID NO: 4, wherein the variation comprises a knob-and-hole mutation and/or removal of Lys (K) at the C-terminal end in SEQ ID NO: 3;
c. a heavy chain having the sequence of SEQ ID NO: 5 or a variant thereof, and a light chain having the sequence of SEQ ID NO: 6, wherein the variation comprises a knob-and-hole mutation and/or removal of Lys (K) at the C-terminal end in SEQ ID NO: 5;
d. a heavy chain having the sequence of SEQ ID NO: 7 or a variant thereof, and a light chain having the sequence of SEQ ID NO: 8, wherein the variation comprises a knob-and-hole mutation and/or removal of Lys (K) at the C-terminal end in SEQ ID NO: 7;
e. a heavy chain having the sequence of SEQ ID NO: 9 or a variant thereof, and a light chain having the sequence of SEQ ID NO: 10, wherein the variation comprises a knob-and-hole mutation and/or removal of Lys (K) at the C-terminal end in SEQ ID NO: 9;
f. a heavy chain having the sequence of SEQ ID NO: 11 or a variant thereof, and a light chain having the sequence of SEQ ID NO: 12, wherein the variation comprises a knob-and-hole mutation and/or removal of Lys (K) at the C-terminal end in SEQ ID NO: 11;
g. a heavy chain having the sequence of SEQ ID NO: 13 or a variant thereof, and a light chain having the sequence of SEQ ID NO: 14, wherein the variation comprises a knob-and-hole mutation and/or removal of Lys (K) at the C-terminal end in SEQ ID NO: 13;
h. a heavy chain having the sequence of SEQ ID NO: 151 or a variant thereof, and a light chain having the sequence of SEQ ID NO: 152, wherein the variation comprises a knob-and-hole mutation and/or removal of Lys (K) at the C-terminal end in SEQ ID NO: 151;
i. a heavy chain having the sequence of SEQ ID NO: 153 or a variant thereof, and a light chain having the sequence of SEQ ID NO: 154, wherein the variation comprises a knob-and-hole mutation and/or removal of Lys (K) at the C-terminal end in SEQ ID NO: 153;
j. a heavy chain having the sequence of SEQ ID NO: 225 or a variant thereof, and a light chain having the sequence of SEQ ID NO: 226, wherein the variation comprises a knob-and-hole mutation and/or removal of Lys (K) at the C-terminal end in SEQ ID 225; or
k. a heavy chain having the sequence of SEQ ID NO: 227 or a variant thereof, and a light chain having the sequence of SEQ ID NO: 228, wherein the variation comprises a knob-and-hole mutation and/or removal of Lys (K) at the C-terminal end in SEQ ID 227.

In some embodiments, the IL-21Rα mutein has at least 10-fold decrease in binding affinity to the IL-21 domain compared to a wild-type IL-21Rα. In some embodiments, the IL-21Rα mutein has 10 to 10,000-fold decrease in binding affinity to the IL-21 domain compared to a wild-type IL-21Rα. In some embodiments, the IL-21Rα mutein has at least 100-fold decrease in binding affinity to the IL-21 domain compared to a wild-type IL-21Rα. In some embodiments, the IL-21Rα mutein has at least 1000-fold decrease in binding affinity to the IL-21 domain compared to a wild-type IL-21Rα. In some embodiments, the IL-21Rα mutein has 10 to 5000-fold decrease in binding affinity to the IL-21 domain compared to a wild-type IL-21Rα. In some embodiments, the IL-21Rα mutein has 100 to 5000-fold decrease in binding affinity to the IL-21 domain compared to a wild-type IL-21Rα. In some embodiments, the IL-21Rα mutein has 100 to 1000-fold decrease in binding affinity to the IL-21 domain compared to a wild-type IL-21Rα. In some embodiments, the IL-21Rα mutein has 500 to 1000-fold decrease in binding affinity to the IL-21 domain compared to a wild-type IL-21Rα. In some embodiments, the IL-21Rα mutein has about 1000-fold decrease in binding affinity to the IL-21 domain compared to a wild-type IL-21Rα. In some embodiments, the IL-21Rα mutein has about 500-fold decrease in binding affinity to the IL-21 domain compared to a wild-type IL-21Rα. In some embodiments, the IL-21Rα mutein has about 100-fold decrease in binding affinity to the IL-21 domain compared to a wild-type IL-21Rα.

In some embodiments, the IL-21Rα mutein has a sequence with at least 95% sequence identity to SEQ ID NO: 15 (IL-21Rα WT). In some embodiments, the IL-21Rα mutein has a sequence with at least 96%, 97%, 98%, or 99% sequence identity to SEQ ID NO: 15 (IL-21Rα WT).

In some embodiments, the IL-21Rα mutein comprises at least one amino acid substitution compared to SEQ ID NO: 15 (IL-21Rα WT). In some embodiments, the IL-21Rα mutein comprises one to five amino acid substitutions compared to SEQ ID NO: 15 (IL-21Rα WT). In some embodiments, the IL-21Rα mutein comprises one amino acid substitution compared to SEQ ID NO: 15 (IL-21Rα WT). In some embodiments, the one or more amino acid substitutions are at one or more amino acid positions selected from Y10, Q35, Y36, E38, L39, F67, H68, M70, A71, D72, D73, I74, L94, P126, Y129, M130, K134, S189, S190, and Y191 of the wild-type IL-21Rα sequence. In some embodiments, the one or more amino acid substitutions are at one or more amino acid positions selected from Y36, E38, L39, M70, A71, D72, D73, I74, and L94 of the wild-type IL-21Rα sequence.

In some embodiments, the amino acid substitutions are selected from:
a. Y10A
b. Q35K, Q35R, or Q35Y;
c. Y36A, Y36C, Y36E, Y36G, Y36H, Y36I, Y36K, Y36M, Y36N, Y36P, Y36Q, Y36R, Y36S, Y36T, or Y36V;
d. E38A, E38C, E38K, E38R, or E38Y;
e. L39A, L39C, L39E, L39F, L39H, L39K, L39R, L39W, or L39Y;
f. F67A;
g. H68A;
h. M70C, M70D, M70F, M70G, M70H, M70K, M70L, M70N, M70Q, M70R, M70S, M70T, M70V, M70W, or M70Y;
i. A71E, A71F, A71I, A71L, A71Q, A71R, A71W, or A71Y;
j. D72A, D72C, D72E, D72F, D72G, D72H, D72I, D72K, D72L, D72M, D72Q, D72R, D72W, or D72Y;
k. D73C, D73A, D73E, D73H, D73K, D73R, D73W, or D73Y;
l. I74A, I74H, I74K, I74R, or I74W;
m. L94A, L94F, L94K, L94Q, L94R, or L94Y;
n. P126A;
o. Y129A;
p. M130A;
q. K134A;
r. 5189A;
s. 5190A; and
t. Y191A.

In some embodiments, the amino acid substitutions are selected from:
a. Y36C, Y36E, Y36G, Y36H, Y36I, Y36K, Y36M, Y36N, Y36P, Y36Q, Y36R, Y36S, Y36T, or Y36V;
b. E38C, E38R, or E38Y;
c. L39A, L39C, L39E, L39F, L39H, L39K, L39R, L39W, or L39Y;
d. M70C, M70D, M70F, M70G, M70H, M70K, M70L, M70N, M70Q, M70R, M70S, M70T, M70V, M70W, or M70Y;
e. A71E, A71F, A71I, A71L, A71Q, A71R, A71W, or A71Y;
f. D72A, D72C, D72E, D72F, D72G, D72H, D72I, D72K, D72L, D72M, D72Q, D72R, D72W, or D72Y;
g. D73A
h. I74R, or I74W; and
i. L94A, L94F, L94K, L94Q, L94R, or L94Y.

In some embodiments, the IL-21Rα mutein comprises a sequence selected from SEQ ID NOs: 18-99 and 155-169.

In some embodiments, the immunocytokine comprises a first chain comprising from the N terminus to C terminus:
a. a Fc fragment of a human IgG1, IgG2, IgG3 or IgG4 having any one sequence selected from SEQ ID NOs: 16, and 185-190; and
b. an IL-21Rα mutein having a sequence selected from SEQ ID NOs: 18-99 and 155-169.

In some embodiments, the immunocytokine comprises a first chain comprising from the N terminus to C terminus:
a. a Fc fragment of a human IgG1, IgG2, IgG3 or IgG4 having any one sequence selected from SEQ ID NOs: 16, and 185-190;
b. a peptide linker; and
c. an IL-21Rα mutein having a sequence selected from SEQ ID NOs: 18-99 and 155-169.

In some embodiments, the immunocytokine comprises a first chain comprising from the N terminus to C terminus:
a. a heavy chain of the ABP comprising a sequence selected from SEQ ID NOs: 1, 3, 5, 7, 9, 11, 13, 151, 153, 225, 227, or a variant thereof; and
b. an IL-21Rα mutein.

In some embodiments, the immunocytokine comprises a first chain comprising from the N terminus to C terminus:
a. a heavy chain of the ABP comprising a sequence selected from SEQ ID NOs: 1, 3, 5, 7, 9, 11, 13, 151, 153, 225, 227, or a variant thereof;
b. a peptide linker; and
c. an IL-21Rα mutein.

In some embodiments, the heavy chain of the ABP comprises a knob variant or a hole variant for knobs-in-holes interaction, wherein the knob variant and the hole variant comprise one or more modifications for the knobs-in-holes interaction.

In some embodiments, the heavy chain of the ABP comprises a variant of the sequence selected from SEQ ID NOs: 1, 3, 5, 7, 9, 11, 13, 151, 153, 225 and 227, wherein the variant has deletion of Lys (K) at the C-terminal end of the sequence.

In some embodiments, the heavy chain of the ABP comprises the sequence of SEQ ID NO: 103.

In some embodiments, the peptide linker is a G45 linker having the sequence of SEQ ID NO: 17. In some embodiments, the peptide linker has a sequence selected from SEQ ID NOs: 212-224.

In some embodiments, the IL-21Rα mutein comprises a sequence selected from SEQ ID NOs: 18-99 and 155-169. In some embodiments, the first chain has a sequence selected from SEQ ID NOs: 104-150 and 192-209.

In some embodiments, the immunocytokine comprises a second chain comprising a heavy chain of the ABP, a peptide linker and the IL-21 domain. In some embodiments, the heavy chain of the ABP comprising a sequence selected from SEQ ID NOs: 1, 3, 5, 7, 9, 11, 13, 151, 153, 225 and 227. In some embodiments, the heavy chain of the ABP comprises a variant of the sequence selected from SEQ ID NOs: 1, 3, 5, 7, 9, 11, 13, 151, 153, 225 and 227. The variant comprises deletion of lysine (Lys or K) at the C-terminal end of the sequence selected from SEQ ID NOs: 1, 3, 5, 7, 9, 11, 13, 151, 153, 225 and 227. In some embodiments, the heavy chain of the ABP may comprise a knob variant or a hole variant for knobs-in-holes interaction. In some embodiments, the peptide linker is selected from SEQ ID NO: 17 and SEQ ID NOs: 212-224. In some embodiments, the second chain has the sequence of SEQ ID NO: 101. In some embodiments, the IL-21 domain is a human IL-21 or a functional variant thereof. In some embodiments, the IL-21 domain has the sequence of SEQ ID NO: 100 (human IL-21).

In some embodiments, the immunocytokine comprises a first heavy chain and a second heavy chain of the ABP. In some embodiments, the first heavy chain comprises a knob mutation and the second heavy chain comprises a hole mutation for knob-and-hole interaction. In some embodiments, the first heavy chain comprises a hole mutation and the second heavy chain comprises a knob mutation for knob-and-hole interaction. In some embodiments, the heavy chain is full length heavy chain or the fragment thereof. In some embodiments, the hole mutation and knob mutation are comprised in a Fc moiety of each heavy chain. In some embodiments, the hole mutation and knob mutation are comprised in a CH3 domain of each heavy chain.

In some embodiments, the immunocytokine comprises a light chain having the sequence of SEQ ID NOs: 2, 4, 6, 8, 10, 12, 14, 102, 152, 154, 226 and 228.

In some embodiments, the IL-21 domain is a human IL-21 or a functional variant thereof. In some embodiments, the IL-21 domain has the sequence of SEQ ID NO: 100 (human IL-21).

In another aspect, the present disclosure provides one or more polynucleotides encoding the immunocytokine provided herein.

In some embodiments, the one or more polynucleotides comprise:
a. a first polynucleotide segment encoding a first chain comprising the heavy chain of the ABP and the IL-21Rα mutein;
b. a second polynucleotide segment encoding a second chain comprising the heavy chain of the ABP and the IL-21 domain; and
c. a third polynucleotide segment encoding the light chain of the ABP.

In some embodiments, the first polynucleotide segment, the second polynucleotide segment, and the third polynucleotide segment are in a single polynucleotide molecule. In some embodiments, the first polynucleotide segment, the second polynucleotide segment, and the third polynucleotide segment are in multiple polynucleotide molecules. In some embodiments, the first polynucleotide segment, the second polynucleotide segment, and the third polynucleotide segment are individually present in separate polynucleotide molecules.

In another aspect, the present disclosure provides one or more vectors comprising the one or more polynucleotides described herein.

In yet another aspect, the present disclosure provides a host cell comprising the one or more polynucleotides or the one or more vectors described herein. In some embodiments, host cell comprises the immunocytokine provided herein. In some embodiments, the host cell is an immune cell. In some embodiments, the immune cell is a T cell.

The host cell can be a eukaryotic cell, for example a fungal cell such as yeast. The host cell can be a mammalian cell (which may be a cell in cell culture, or a cell present in a tissue or organ). In some embodiments, the host cell is a human, mouse, rat, rabbit, bovine or dog (or, for example, any other wild, livestock/domesticated animal) cell. In some embodiments, the host cell is a stable cell line cell, or a primary cell, adherent or suspension cell. As examples, the host cell can be a macrophage, osteosarcoma, or CHO, BHK (baby hamster kidney), Bowes human melanoma cell, 911, AT1080, A549, HEK293, or HeLa cell line cell or a mouse primary cell, but not limited thereto. In some embodiments, the host cell is a bacterial cell, such as E. coli.

The eukaryotic cell can be a plant cell (for example a monocotyledonous or dicotyledonous plant cell; typically an experimental, crop and/or ornamental plant cell, for example Arabidopsis, maize); fish (for example Zebra fish; salmon), bird (for example chicken or other domesticated bird), insect (for example Drosophila; bees), Nematoidia or Protista (for example Plasmodium spp or Acantamoeba spp) cell.

In one aspect, the present disclosure provides a method of enhancing immune response in a subject, comprising administration of the immunocytokine described herein or the host cell described herein to the subject. In some embodiments, the subject is a cancer patient.

In one aspect, the present disclosure provides a method of selectively activating an IL-21Rα on a target cell, comprising: delivering the immunocytokine of the present disclosure to the target cell. In some embodiments, the target cell is an immune cell. In some embodiments, the immune cell is a T cell.

Another aspect of the present disclosure provides an IL-21Rα mutein having a reduced binding affinity to an IL-21 domain compared to a wild-type IL-21Rα.

In some embodiments, the wild-type IL-21Rα comprises the sequence of SEQ ID NO: 15. In some embodiments, the IL-21 domain is a human IL-21 or a functional variant thereof. In some embodiments, the IL-21 domain has the sequence of SEQ ID NO: 100.

In some embodiments, the IL-21Rα mutein has at least 10-fold decrease in binding affinity to the IL-21 domain compared to a wild-type IL-21Rα. In some embodiments, the IL-21Rα mutein has 10 to 10,000-fold decrease in binding affinity to the IL-21 domain compared to a wild-type IL-21Rα. In some embodiments, the IL-21Rα mutein has at least 100-fold decrease in binding affinity to the IL-21 domain compared to a wild-type IL-21Rα. In some embodiments, the IL-21Rα mutein has at least 1000-fold decrease in binding affinity to the IL-21 domain compared to a wild-type IL-21Rα. In some embodiments, the IL-21Rα mutein has 10 to 5000-fold decrease in binding affinity to the IL-21 domain compared to a wild-type IL-21Rα. In some embodiments, the IL-21Rα mutein has 100 to 5000-fold decrease in binding affinity to the IL-21 domain compared to a wild-type IL-21Rα. In some embodiments, the IL-21Rα mutein has 100 to 1000-fold decrease in binding affinity to the IL-21 domain compared to a wild-type IL-21Rα. In some embodiments, the IL-21Rα mutein has 500 to 1000-fold decrease in binding affinity to the IL-21 domain compared to a wild-type IL-21Rα.

In some embodiments, the IL-21Rα mutein has about 1000-fold decrease in binding affinity to the IL-21 domain compared to a wild-type IL-21Rα. In some embodiments, the IL-21Rα mutein has about 500-fold decrease in binding affinity to the IL-21 domain compared to a wild-type IL-21Rα. In some embodiments, the IL-21Rα mutein has about 100-fold decrease in binding affinity to the IL-21 domain compared to a wild-type IL-21Rα.

In some embodiments, the IL-21Rα mutein has a sequence with at least 95% sequence identity to SEQ ID NO: 15 (IL-21Rα WT). In some embodiments, the IL-21Rα mutein has a sequence with at least 96%, 97%, 98%, or 99% sequence identity to SEQ ID NO: 15 (IL-21Rα WT).

In some embodiments, the IL-21Rα mutein comprises at least one amino acid substitution compared to SEQ ID NO: 15 (IL-21Rα WT). In some embodiments, the IL-21Rα mutein comprises one to five amino acid substitutions compared to SEQ ID NO: 15 (IL-21Rα WT). In some embodiments, the IL-21Rα mutein has one amino acid substitution compared to SEQ ID NO: 15 (IL-21Rα WT).

In some embodiments, the one or more amino acid substitutions are at one or more amino acid positions selected from Y10, Q35, Y36, E38, L39, F67, H68, M70, A71, D72, D73, I74, L94, P126, Y129, M130, K134, S189, S190, and Y191 of the wild-type IL-21Rα sequence. In some embodiments, the one or more amino acid substitutions are at one or more amino acid positions selected from Y36, E38, L39, M70, A71, D72, D73, I74, and L94 of the wild-type IL-21Rα sequence.

In some embodiments, the amino acid substitutions are selected from:
a. Y10A
b. Q35K, Q35R, or Q35Y;
c. Y36A, Y36C, Y36E, Y36G, Y36H, Y36I, Y36K, Y36M, Y36N, Y36P, Y36Q, Y36R, Y36S, Y36T, or Y36V;
d. E38A, E38C, E38K, E38R, or E38Y;
e. L39A, L39C, L39E, L39F, L39H, L39K, L39R, L39W, or L39Y;
f. F67A;
g. H68A;
h. M70C, M70D, M70F, M70G, M70H, M70K, M70L, M70N, M70Q, M70R, M70S, M70T, M70V, M70W, or M70Y;
i. A71E, A71F, A71I, A71L, A71Q, A71R, A71W, or A71Y;
j. D72A, D72C, D72E, D72F, D72G, D72H, D72I, D72K, D72L, D72M, D72Q, D72R, D72W, or D72Y;
k. D73C, D73A, D73E, D73H, D73K, D73R, D73W, or D73Y;
l. I74A, I74H, I74K, I74R, or I74W;
m. L94A, L94F, L94K, L94Q, L94R, or L94Y;
n. P126A;
o. Y129A;
p. M130A;
q. K134A;
r. S189A;
s. S190A; and
t. Y191A.

In some embodiments, the amino acid substitutions are selected from:
a. Y36C, Y36E, Y36G, Y36H, Y36I, Y36K, Y36M, Y36N, Y36P, Y36Q, Y36R, Y36S, Y36T, or Y36V;
b. E38C, E38R, or E38Y;
c. L39A, L39C, L39E, L39F, L39H, L39K, L39R, L39W, or L39Y;
d. M70C, M70D, M70F, M70G, M70H, M70K, M70L, M70N, M70Q, M70R, M70S, M70T, M70V, M70W, or M70Y;
e. A71E, A71F, A71I, A71L, A71Q, A71R, A71W, or A71Y;
f. D72A, D72C, D72E, D72F, D72G, D72H, D72I, D72K, D72L, D72M, D72Q, D72R, D72W, or D72Y;
g. D73A
h. I74R, or I74W; and
i. L94A, L94F, L94K, L94Q, L94R, or L94Y.

In some embodiments, the IL-21Rα mutein comprises a sequence selected from SEQ ID NOs: 18-99 and 155-169.

In another aspect, the present disclosure provides a polynucleotide comprising a coding sequence of the IL-21 Rα mutein described herein. In yet another aspect, the present disclosure provides a vector comprising the polynucleotide. In some embodiments, the vector is a viral vector. In some embodiments, the vector is a recombinant AAV or lentiviral vector.

The present disclosure also provides a host cell comprising the IL-21 Rα mutein, the polynucleotide, or the vector described herein.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 provides a schematic representation of an exemplary immunocytokine (αPD-1IL21RαMutein/IL21).

FIGS. 2A-2V provide sensorgrams from SPR full kinetics assay of IL-21Rα muteins against IL21.

FIG. 3 provides experimental results testing 66 different αPD-1IL21RαMutein/IL21. X-axis shows the affinity of immunocytokines to IL21 measured by Bio Layer interferometry (BLI) and y-axis shows efficacy of IL-21 mediated STAT3 phosphorylation (efficacy coefficient). The results show that as IL21Rα mutein in the immunocytokine has a reduced binding affinity to IL-21, the immunocytokine (αPD-1IL21RαMutein/IL21) has a higher efficacy coefficient.

FIG. 4 provides concentration dependent curves of selected six αPD-1IL21RαMutein/IL21, αPD-1IL21RαWT/IL21 (“WT”) and recombinant human IL-21 protein (“IL-21”). Specifically, the graph shows IL-21 mediated activation (efficacy coefficient) in PD-1(+) H9 cells. The max potency of αPD-1IL21RαMutein/IL21 with M70Q and M70H mutation was comparable to the recombinant human IL-21 (“IL-21”), and the others showed at least above 80%.

FIG. 5 provides concentration dependent curves of selected six αPD-1IL21RαMutein/IL21, αPD-1IL21RαWT/IL21 (“WT”) and recombinant human IL-21 protein (“IL-21”). Specifically, the graph shows IL-21 mediated activation (efficacy coefficient) in PD-1(−) H9 cells. The max potency of six αPD-1IL21RαMutein/IL21 was similar to the recombinant human IL-21 (“IL-21”), but EC50 increased in all variants.

FIGS. 6A-6E provide response curve of STAT3 phosphorylation observed in PD-1(−) H9 cells and PD-1(+) H9 cells in response to 16 variants of αPD-1IL21RαMutein/IL21.

FIGS. 7A-7Q provide sensorgrams from SPR full kinetics assay of immunocytokines (αPD-1IL21RαMutein/IL21) against PD-1.

FIG. 8A shows measurements of the binding affinities of the αPD-1 antibody or Immunocytokine to FcRn using ForteBio Octet RED96e instruments. FIG. 8B is a table summarizing binding kinetics of the αPD-1 antibody or Immunocytokine to FcRn.

FIGS. 9A, 9B and 9C provide sensorgrams data from SPR full kinetics assay of immunocytokines (αCTLA-4IL21RαMutein/IL21; αTIGITIL21RαMutein/IL21; or αLAG-3IL21RαMutein/IL21) against their targets (hCTLA-4, hTIGIT, or hLAG-3).

FIG. 10 shows IFNγ concentrations (pg/ml) released from CTLs (effector cells) co-cultured with immunocytokines (αPD-1IL21RαMutein/IL21 including M70D, M70Q, L94K and E38R) as described in Section 5.7. The data are compared against IFNγ release in response to αPD-1 antibody or αPD-1IL21RαWT/IL21 (“WT”).

FIGS. 11A and 11B show fluorescent signals (RFU) of Calcein AM released from dead tumor cells as described in Section 5.7.2. The signal indicates tumor killing efficacy of effector cells against tumor cells (MeWo cell line (FIG. 11A) and A375_CMV cell line (FIG. 11B)) treated with immunocytokines (αPD-1IL21RαMutein/IL21 including M70D, M70Q, L94K and E38R) or controls (αPD-1 antibody or αPD-1IL21RαWT/IL21 (“WT”)).

FIG. 12 provides STAT3 phosphorylation curves obtained from HTRF-based high-throughput assay described in Section 5.9. It shows STAT3 phosphorylation induced by rhIL21, ABP-IL21RαWT/IL21, and ABP-IL21RαMutein/IL21, but not by antibodies without IL21 conjugation (i.e., anti-CTLA-4 antibody (Ipilimumab), anti-TIGIT antibody (Tiragolumab), anti-LAG-3 antibody (Relatlimab)).

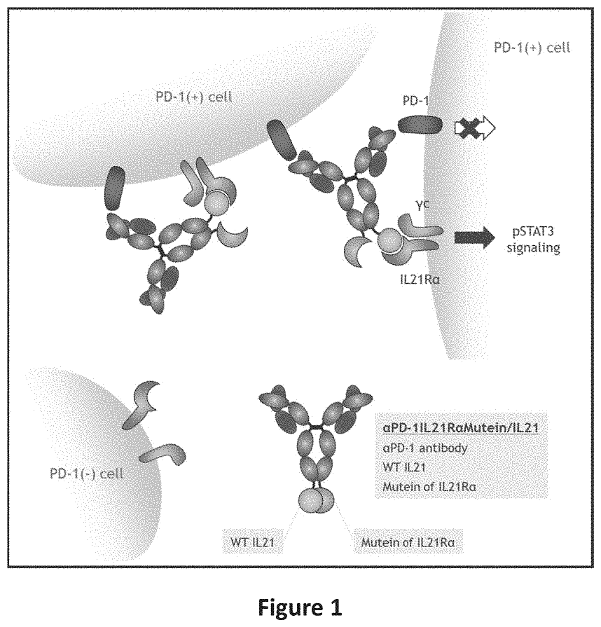

### 6. DETAILED DESCRIPTION OF THE INVENTION

6.1. Definitions

The term “IL-21Rα mutein”, “IL-21RαMutein”, “IL21Rα mutein” or “IL21RαMutein” as used herein refers to the ectodomain of an IL-21Rα having one or more modifications. The modifications can be amino acid substitution, insertion, deletion or other mutation. In some embodiments, IL-21Rα mutein includes one or more biological, chemical, or both modifications compared to wild-type human IL-21Rα or its ectodomain. In some embodiments, the ectodomain of the wild-type human IL-21Rα comprises the sequence of SEQ ID NO: 15.

The term “pharmaceutically acceptable carrier” as used herein refers to a carrier or diluent that does not impair the biological activity and characteristics of an immunocytokine according to the present invention. As a pharmaceutically acceptable carrier in a composition that is formulated as a liquid solution, a sterile and biocompatible carrier can be used. The pharmaceutically acceptable carrier can be physiological saline, sterile water, Ringer's solution, buffered saline, albumin injection solution, dextrose solution, maltodextrin solution, glycerol, ethanol, or a mixture of two or more thereof. In addition, the composition of the present invention may, if necessary, comprise other conventional additives, including antioxidants, buffers, and bacteriostatic agents. Further, the composition of the present invention can be formulated as injectable forms such as aqueous solutions, suspensions or emulsions with the aid of diluents, dispersants, surfactants, binders and lubricants. In addition, the composition according to the present invention can be formulated in the form of pills, capsules, granules, or tablets. Other carriers known in the art, e.g., as described in a literature [Remington's Pharmaceutical Sciences (E. W. Martin)], can be used.

The term “antigen-binding protein (ABP)” refers to a protein comprising one or more antigen-binding domains that specifically bind to an antigen or epitope. In some embodiments, the antigen-binding domain binds the antigen or epitope with specificity and affinity similar to that of naturally occurring antibodies. In some embodiments, the ABP comprises an antibody. In some embodiments, the ABP consists of an antibody. In some embodiments, the ABP consists essentially of an antibody. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. In some embodiments, the ABP binds the extracellular domain of the target protein. In certain embodiments, the ABP provided herein binds to an epitope of the target protein that is conserved between or among various species.

In some embodiments, the ABP is an antibody and the antibody can be a monoclonal antibody, a polyclonal antibody, a multi-specific antibody, a dual-specific or bispecific antibody, an anti-idiotypic antibody, or a bifunctional hybrid antibody. In some embodiments, the ABP comprises one or more heavy chain or a fragment thereof. In some embodiments, the ABP comprises one or more light chain or a fragment thereof. In some embodiments, the antibody comprises two heavy chains and two light chains, or fragments thereof. In some embodiments, the fragment of the heavy chain comprises Fc fragment, CH3 domain, or CH2 domain of the heavy chain.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of naturally occurring antibodies. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), diabody, gamma-B-crystallin/ubiquitin (e.g., Affilins), CTLD₃ (e.g., Tetranectins), Fynomers, and LDLR-A module (e.g., Avimers). Additional information on alternative scaffolds is provided in Binz et al., Nat. Biotechnol., 2005 23:1257-1268; Skerra, Current Opin. In Biotech., 2007 18:295-304; and Silacci et al., J. Biol. Chem., 2014, 289:14392-14398; each of which is incorporated by reference in its entirety. An alternative scaffold is one type of ABP.

The term “antibody fragment” comprises a portion of an intact antibody, such as the antigen-binding or variable region of an intact antibody. Antibody fragments include, for example, Fv fragments, antigen-binding fragments (Fab), F(ab′)₂ fragments, Fab′ fragments, single chain variable fragments (scFv, sFv), scFv-Fc fragments. Disulfide-linked Fv fragments, and a single domain antibody (sdAb).

The term “antigen-binding domain” means the portion of an ABP that is capable of specifically binding to an antigen or epitope.

The term “Fe fragment” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125: S41-52, incorporated by reference in its entirety. The Fc fragment can comprise two Fc moieties. The Fc moiety can comprise a CH2-CH3 domain of a heavy chain. In some embodiments, the ABP comprises an Fc fragment comprising two Fc moieties, wherein each Fc moiety is independently selected from IgG subclasses, e.g., IgG1, IgG2, IgG3, and IgG4. In some embodiments, the ABP comprises two Fc moieties of IgG1. In some embodiments, the ABP comprises two Fc moieties of IgG4. In some embodiments, the ABP comprises an Fc fragment comprising two Fc moieties, wherein the first Fc moiety is an Fc moiety of IgG1 and the second Fc moiety is an Fc moiety of IgG4. In some embodiments, the ABP comprises an Fc fragment comprising two Fc moieties, wherein the first Fc moiety comprises an C_{H}3 of IgG1 and the second Fc moiety comprises an C_{H}3 of IgG4.

The Fc region may be a naturally occurring Fc region, or an Fc region modified as described elsewhere in this disclosure. For example, the Fc moiety can be a knob variant or a hole variant for knobs-in-holes interaction. The Fc fragment can comprise a knob variant and a hole variant of a C-terminal region of an immunoglobulin heavy chain.

In some cases, the Fc fragment is engineered to introduce mutations to reduce effector function of immunoglobulin, which minimize ADCC by reducing the binding affinity for FcγR. Those mutations are the so-called LALA mutation(L234A/L235A) for human IgG1 type and SPLE mutation (S228P/L235E). (see, e.g., Hezareh et al. J. Virol. (2001) 75(24): 12161-8). In further embodiments, the LALA or SPLE mutations are present in the Fc fragment with the knobs-into-holes mutations.

The Fc fragment can comprise the M252Y/S254T/T256E (“YTE”) mutations. The YTE mutations allow the simultaneous modulation of serum half-life, tissue distribution and activity of IgG₁ (see DalFAcqua et al., J Biol Chem. (2006) 281:23514-24; and Robbie et al., Antimicr oh Agents Chemother. (2013) 57(12):6147-53). In further embodiments, the YTE mutations are present in the antibody with the knobs-into-holes mutations.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5^{th} ed. (1991) Public Health Service, National Institutes of Health, Bethesda, Md., incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“Aho” numbering scheme); each of which is incorporated by reference in its entirety.

Table 1 provides exemplary positions of CDR1-L (CDR1 of V_{L}), CDR2-L (CDR2 of V_{L}), CDR3-L (CDR3 of V_{L}), CDR1-H (CDR1 of V_{H}), CDR2-H (CDR2 of V_{H}), and CDR3-H (CDR3 of V_{H}), as identified by the Kabat and Chothia schemes. For CDR1-H, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839 or bioinf.org.uk—Prof. Andrew C. R. Martin's group at UCL, incorporated by reference in its entirety.

TABLE 1
Exemplary CDR residues according to
Kabat and Chothia numbering schemes.

| CDR | Kabat | Chothia |
|---|---|---|
| CDR1-L | 24-34 | 24-34 |
| CDR2-L | 50-56 | 50-56 |
| CDR3-L | 89-97 | 89-97 |
| CDR1-H (Kabat Numbering) | 31-35B | 26-32 or 34* |
| CDR1-H (Chothia Numbering) | 31-35 | 26-32 |
| CDR2-H | 50-65 | 52-56 |
| CDR3-H | 95-102 | 95-102 |

*The C-terminus of CDR1-H, when numbered using the Kabat numbering convention, varies between 32 and 34, depending on the length of the CDR.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing metastasis, decreasing the rate of disease progression, amelioration or palliation of the disease state, and remission or improved prognosis.

6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

6.3. Summary of Experimental Observation

The present disclosure provides IL21RαMutein having a reduced affinity to IL-21 compared to IL21RαWT, and immunocytokines comprising the IL21RαMutein as a capping moiety.

As one example, the present disclosure provides an immunocytokine (αPD-1IL21RαMutein/IL21) comprising an ABP targeting PD-1. The immunocytokine can be targeted to PD-1 expressing cells, such as CD4+ or CD8 T+ cells. The immunocytokine comprises four polypeptide chains—two identical light chains and two different heavy chains—joined to form a heterodimer by knobs-into-holes (KiH) interaction. In the immunocytokine, one of the two heavy chains is fused to IL-21 and the other one is fused to a capping moiety, which is a mutant of ectodomain of IL-21Rα (IL-21RαMutein). IL-21 and the capping moiety are fused to the heavy chains through a non-cleavable and flexible polypeptide linker.

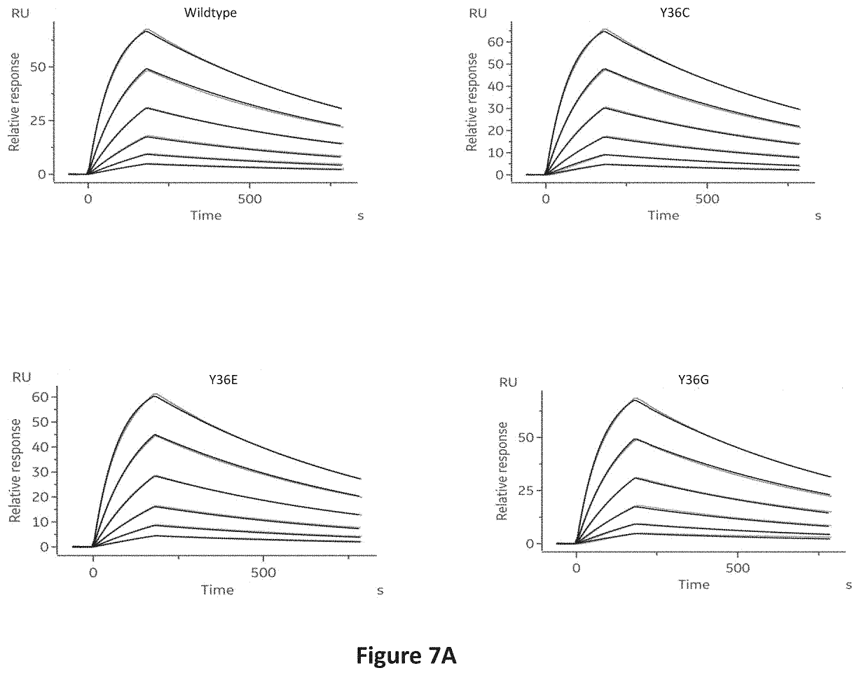

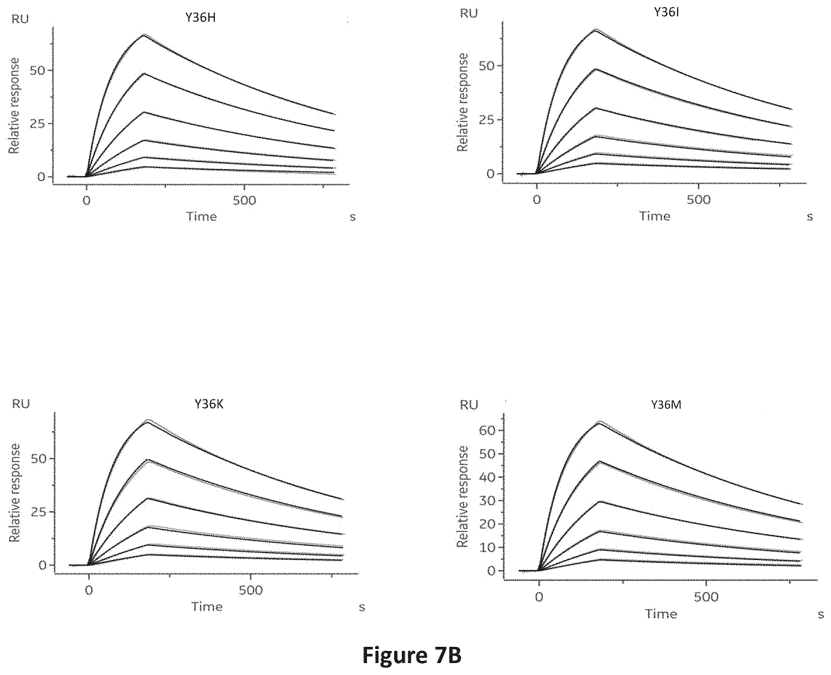

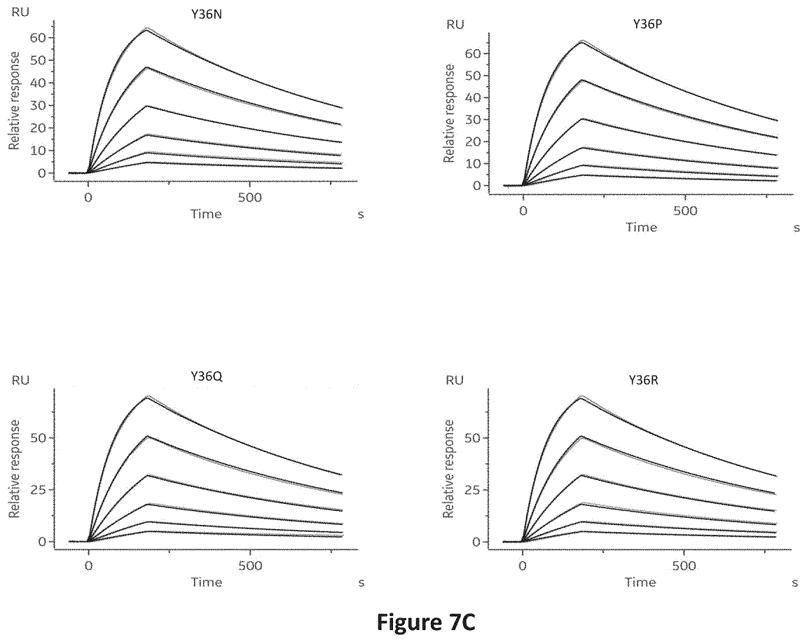

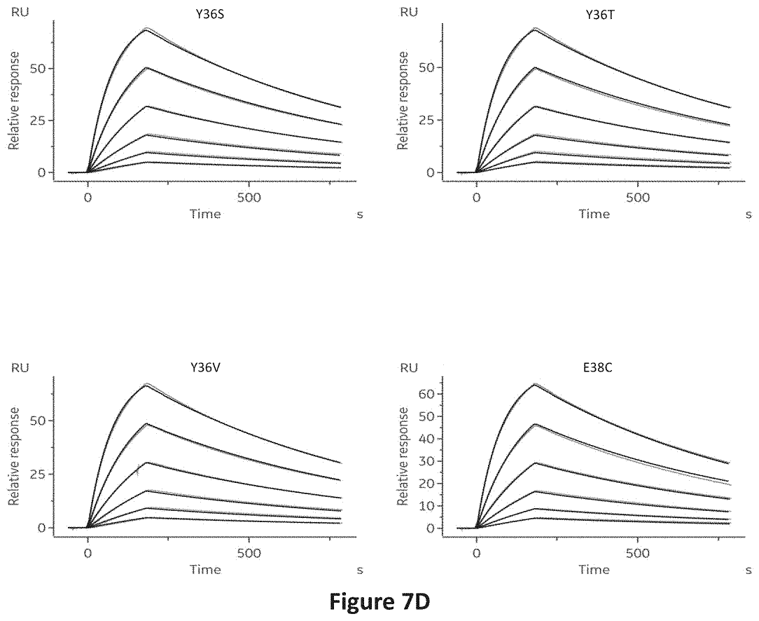

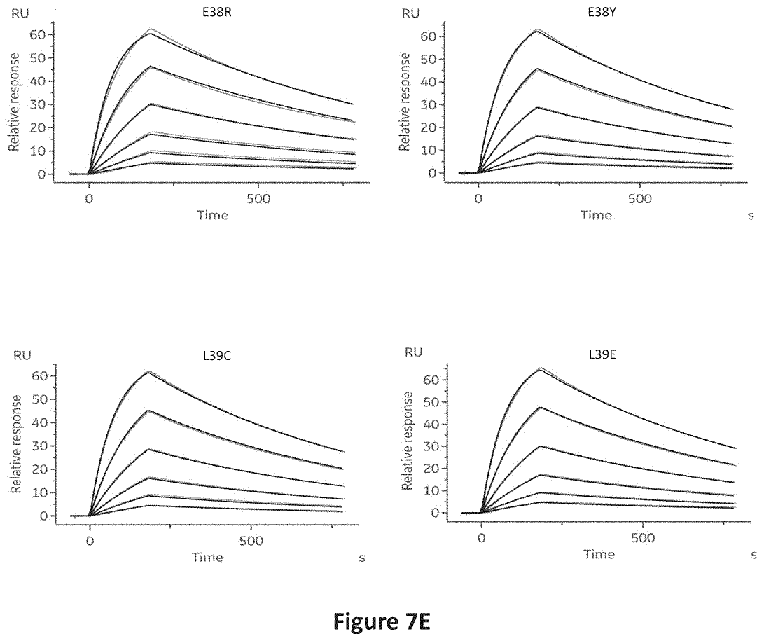

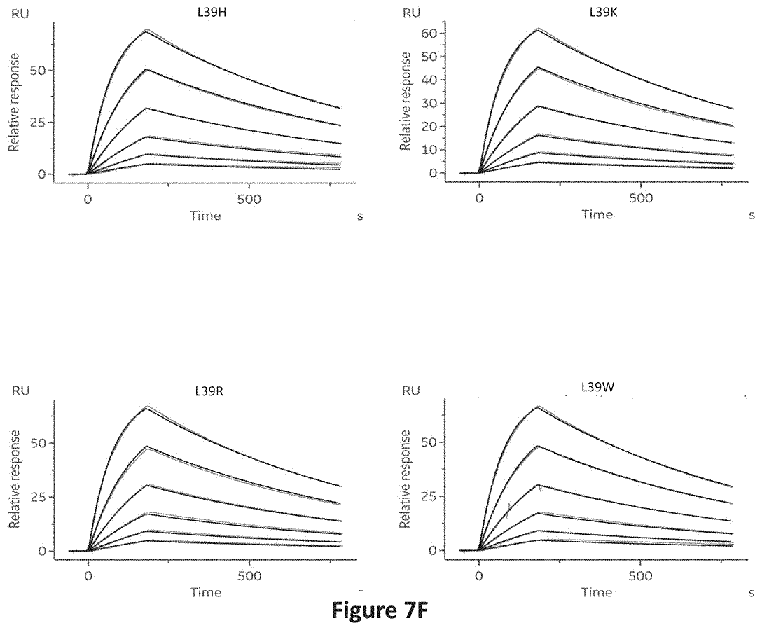

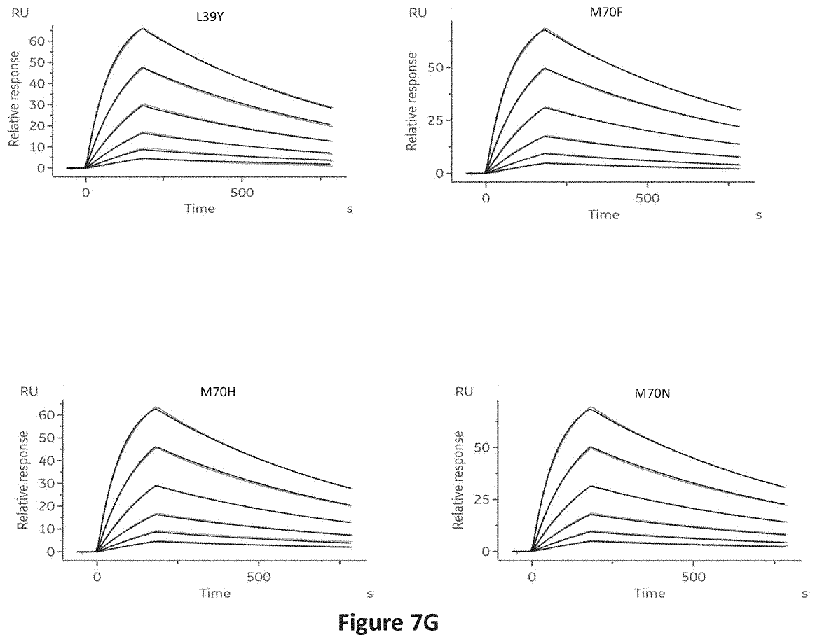

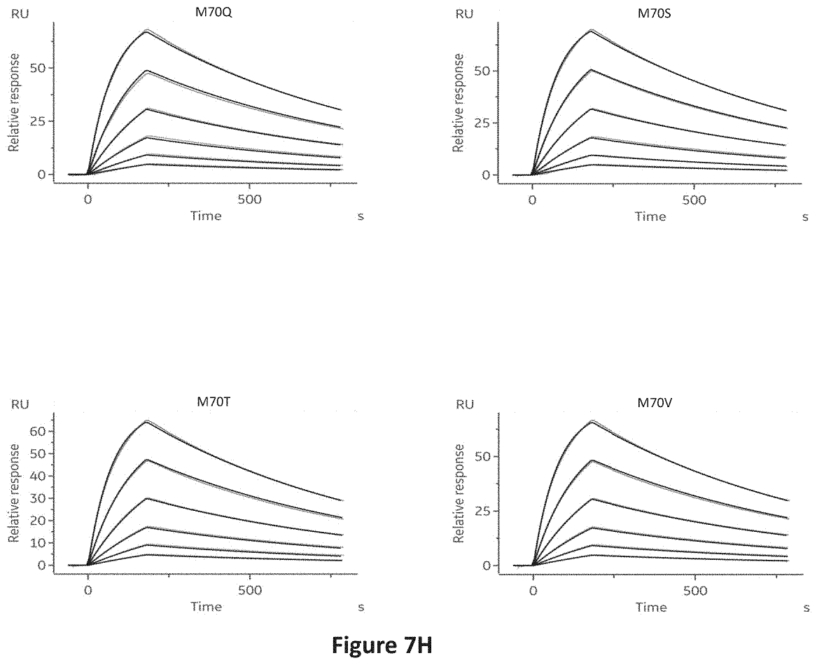

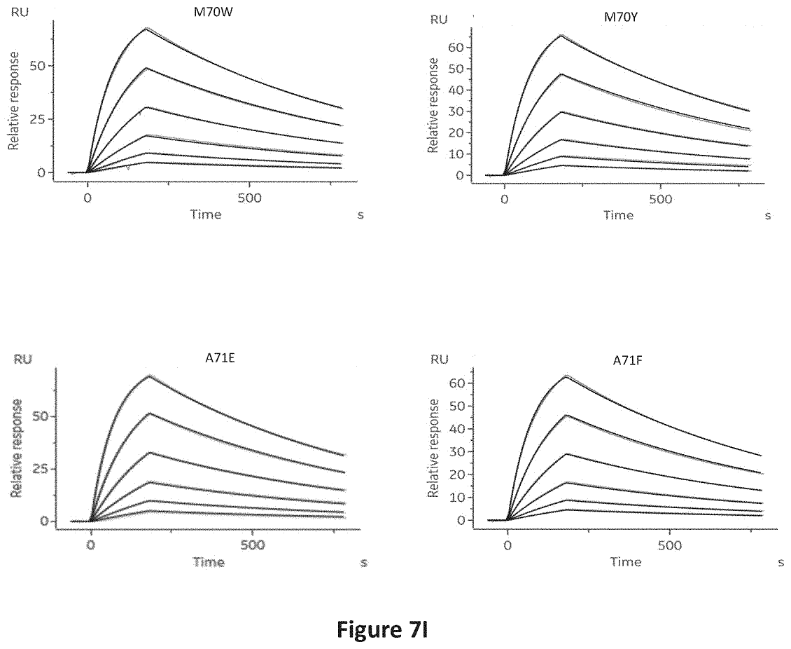

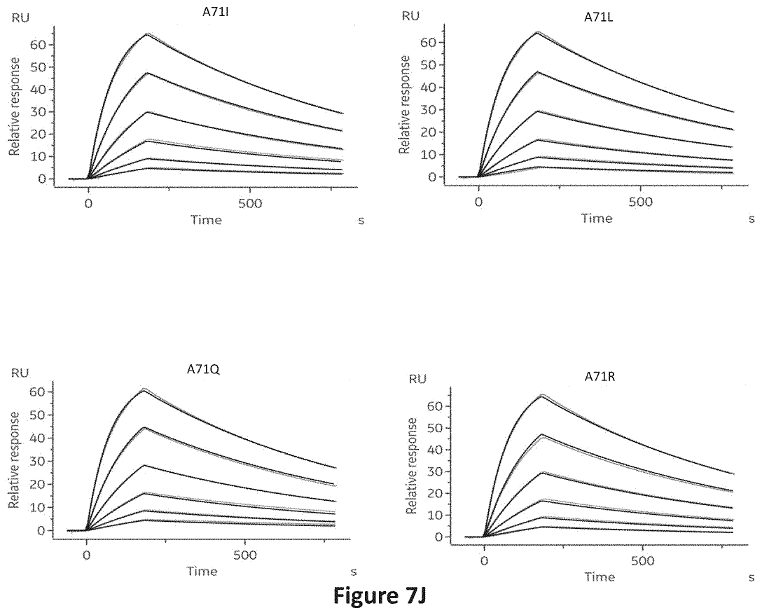

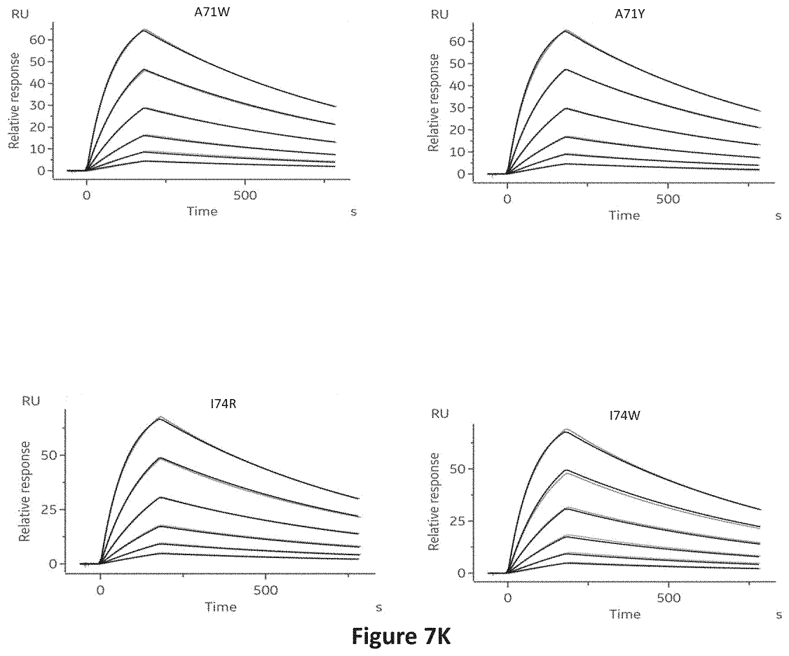

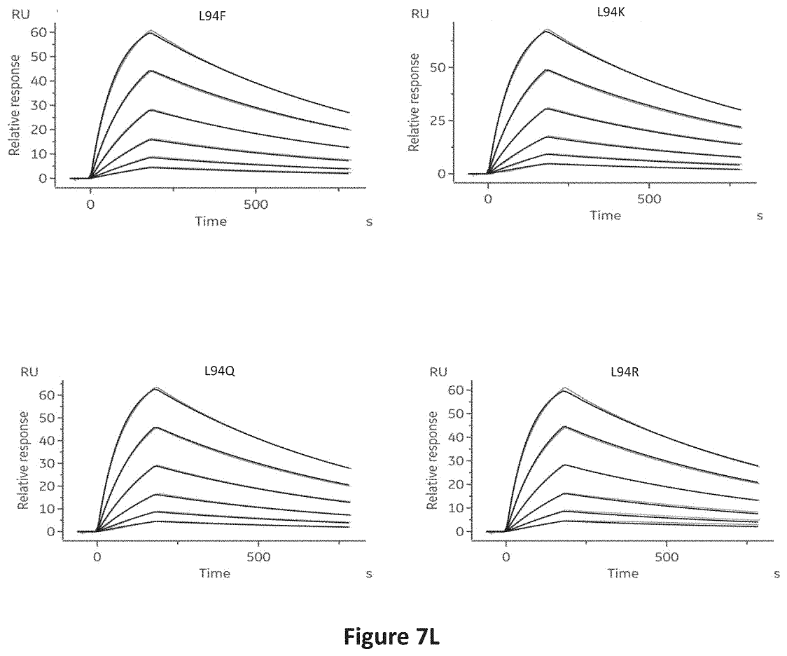

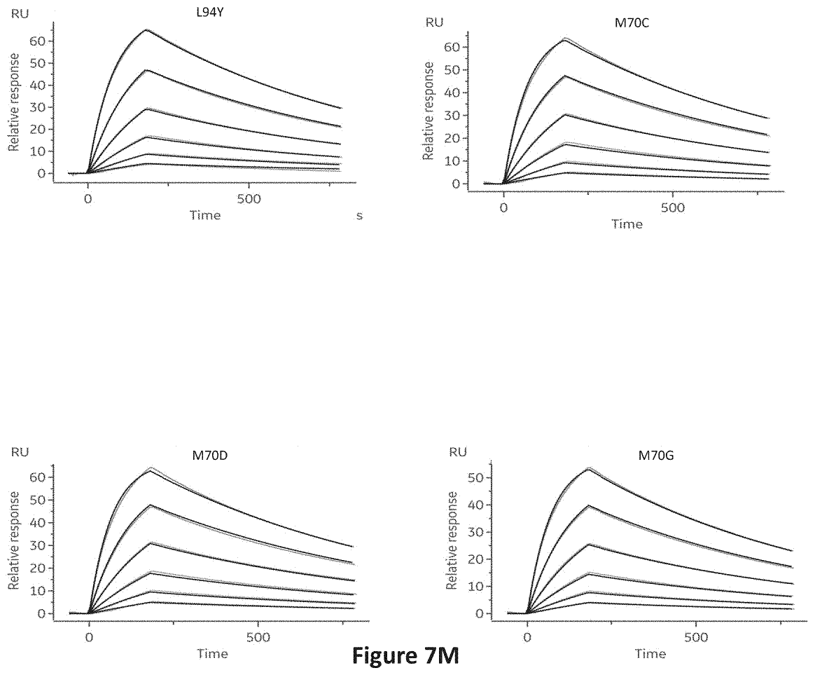

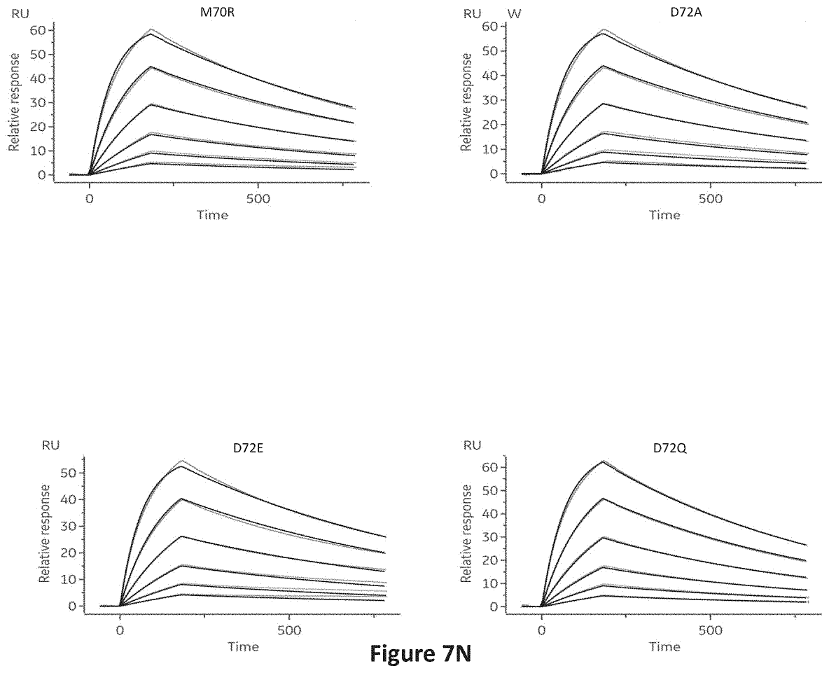

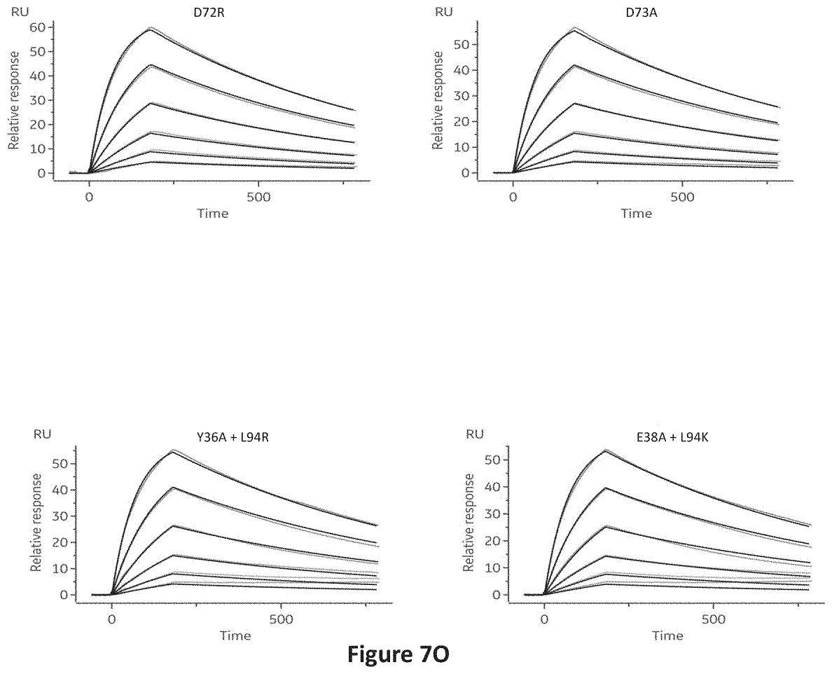

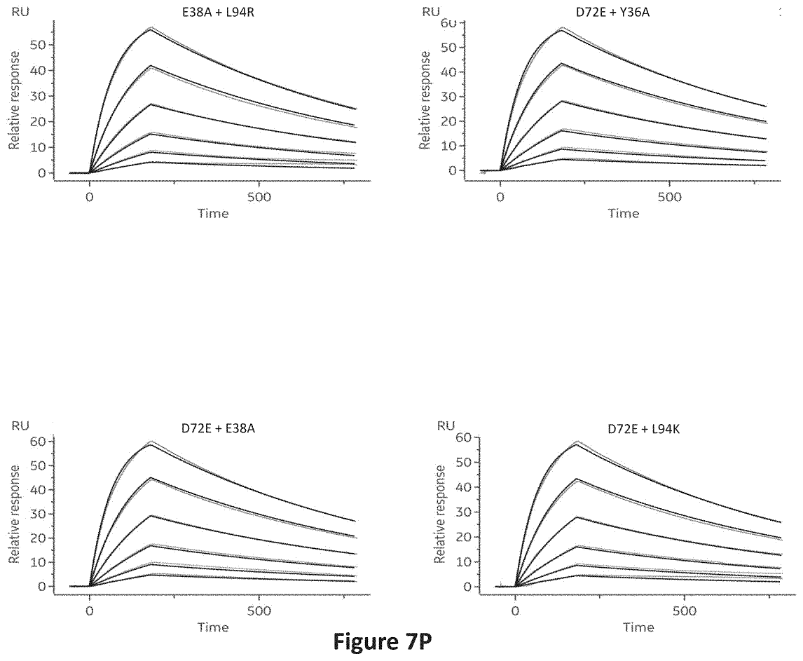

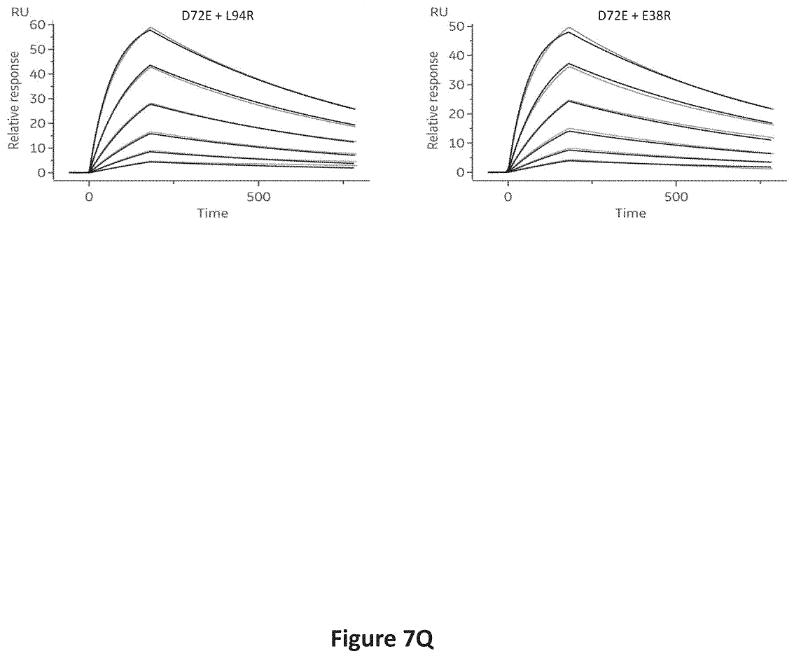

Applicant expressed the immunocytokines in CHO cells and purified them with a purity of ≥95%. The immunocytokine had 185 kDa molecular weight in the de-glycosylated form and 195 kDa in the glycosylated form when measured by mass spectrometry. Applicant also confirmed that over 90% of the molecules were present in the heterodimeric form of αPD-1IL21RαMutein/IL21. Applicant further measured activity of anti-PD-1 antibody using the PD-L1/TCR activator-CHO recombinant cell line (BPS bioscience) which can measure the intensity of TCR signaling through a luciferase reporter system driven by an NFAT-response element. The experiment showed that the fusion of IL21RαMutein/IL21 to IgG had little effect on the activity of anti-PD-1 antibody. Furthermore, SPR analysis demonstrated that the fusion of IL-21WT or IL21RαMutein and IL-21 to the anti-PD-1 antibody did not affect the affinity to PD-1 (FIG. 7A-7Q).

Applicant generated 66 candidates of immunocytokine comprising anti-PD-1 IgG, IL-21, and one of various muteins of IL-21Rα. Next, using a high throughput HTRF assay, Applicant tested whether application of αPD-1IL21RαMutein/IL21 increases phosphorylation of STAT3 in PD-1(+) T cell. Based on the results from the HTRF assay, six αPD-1IL21RαMutein/IL21 candidates were selected. The selected candidates showed max potency at lower concentration compared to control αPD-1IL21RαWT/IL21 treatment and acted selectively on PD-1(+) cells. They showed superior potency at lower concentration compared to the control immunocytokine (αPD-1IL21RαWT/IL21).

Anti-cancer efficacy of the selected candidates can be tested in a humanized PDX mouse model. When αPD-1IL21RαMutein/IL21 binds to PD-1 expressed on PD-1(+) T cells, the reduced binding affinity of IL21RαMutein to human IL-21 can allow IL-21 of the immunocytokine to compete with and bind to endogenous IL21Rα (e.g., IL21RαWT), and lead to the invigoration of PD-1(+) T cells for the generation of durable anti-cancer immunity.

In summary, the present disclosure provides an immunocytokine that can exclusively deliver IL-21 to PD-1(+) T cells and reinvigorate the T cells to acquire a memory-like phenotype for long-lasting anti-cancer immunity.

6.4. IL-21Rα Muteins

In one aspect, the present disclosure provides IL-21Rα muteins having a reduced binding affinity to an IL-21 domain compared to a wild-type IL-21Rα. In some embodiments, the IL-21Rα mutein has a mutation at the binding site of IL-21Rα against IL-21. In some embodiments, the IL-21Rα mutein has one or more amino acid substitution, insertion, or deletion at a binding site of IL-21Rα against IL-21.

In some embodiments, the IL-21Rα mutein specifically binds to the IL-21 domain, but with a reduced affinity. In some embodiments, the IL-21Rα mutein has at least 10-fold decrease in binding affinity to the IL-21 domain compared to a wild-type IL-21Rα. In some embodiments, the IL-21Rα mutein has at least 50-fold decrease in binding affinity to the IL-21 domain compared to a wild-type IL-21Rα. In some embodiments, the IL-21Rα mutein has at least 100-fold decrease in binding affinity to the IL-21 domain compared to a wild-type IL-21Rα. In some embodiments, the IL-21Rα mutein has at least 200-fold decrease in binding affinity to the IL-21 domain compared to a wild-type IL-21Rα. In some embodiments, the IL-21Rα mutein has at least 300-fold decrease in binding affinity to the IL-21 domain compared to a wild-type IL-21Rα. In some embodiments, the IL-21Rα mutein has at least 500-fold decrease in binding affinity to the IL-21 domain compared to a wild-type IL-21Rα. In some embodiments, the IL-21Rα mutein has at least 1000-fold decrease in binding affinity to the IL-21 domain compared to a wild-type IL-21Rα. In some embodiments, the IL-21Rα mutein has at least 5000-fold decrease in binding affinity to the IL-21 domain compared to a wild-type IL-21Rα.

In some embodiments, the IL-21Rα mutein has 10 to10,000-fold decrease in binding affinity to the IL-21 domain compared to a wild-type IL-21Rα. In some embodiments, the IL-21Rα mutein has 10 to 5,000-fold decrease in binding affinity to the IL-21 domain compared to a wild-type IL-21Rα. In some embodiments, the IL-21Rα mutein has 100 to 5,000-fold decrease in binding affinity to the IL-21 domain compared to a wild-type IL-21Rα. In some embodiments, the IL-21Rα mutein has 10 to 1,000-fold decrease in binding affinity to the IL-21 domain compared to a wild-type IL-21Rα. In some embodiments, the IL-21Rα mutein has 100 to 1,000-fold decrease in binding affinity to the IL-21 domain compared to a wild-type IL-21Rα. In some embodiments, the IL-21Rα mutein has 500 to 1,000-fold decrease in binding affinity to the IL-21 domain compared to a wild-type IL-21Rα. In some embodiments, the IL-21Rα mutein has 500 to 2,000-fold decrease in binding affinity to the IL-21 domain compared to a wild-type IL-21Rα. In some embodiments, the IL-21Rα mutein has 1,000 to 2,000-fold decrease in binding affinity to the IL-21 domain compared to a wild-type IL-21Rα. In some embodiments, the IL-21Rα mutein has 2,000 to 5000-fold decrease in binding affinity to the IL-21 domain compared to a wild-type IL-21Rα.

In some embodiments, the IL-21Rα mutein has about 5000-fold decrease in binding affinity to the IL-21 domain compared to a wild-type IL-21Rα. In some embodiments, the IL-21Rα mutein has about 2500-fold decrease in binding affinity to the IL-21 domain compared to a wild-type IL-21Rα. In some embodiments, the IL-21Rα mutein has about 1000-fold decrease in binding affinity to the IL-21 domain compared to a wild-type IL-21Rα. In some embodiments, the IL-21Rα mutein has about 500-fold decrease in binding affinity to the IL-21 domain compared to a wild-type IL-21Rα. In some embodiments, the IL-21Rα mutein has about 100-fold decrease in binding affinity to the IL-21 domain compared to a wild-type IL-21Rα.

In some embodiments, the wild-type IL-21Rα is the ectodomain of a human IL-21Rα. In some embodiments, the wild-type IL-21Rα has the sequence of SEQ ID NO: 15. In some embodiments, the IL-21Rα mutein has a sequence with at least 95% sequence identity to SEQ ID NO: 15. In some embodiments, the IL-21Rα mutein has a sequence with at least 96% sequence identity to SEQ ID NO: 15. In some embodiments, the IL-21Rα mutein has a sequence with at least 97% sequence identity to SEQ ID NO: 15. In some embodiments, the IL-21Rα mutein has a sequence with at least 98% sequence identity to SEQ ID NO: 15. In some embodiments, the IL-21Rα mutein has a sequence with at least 99% sequence identity to SEQ ID NO: 15.

In some embodiments, the IL-21 Rα mutein includes one or more modifications at a binding site involved in the interaction between IL-21 and IL-21 Rα. In some embodiments, the one or more modifications are amino acid substitution, deletion, insertion, or a combination thereof. In some embodiments, the one or more modifications are chemical modifications. In some embodiments, the modifications can induce structural change in the binding site.

In some embodiments, the IL-21Rα mutein comprises at least one amino acid substitution compared to SEQ ID NO: 15. In some embodiments, the IL-21Rα mutein comprises one amino acid substitution compared to SEQ ID NO: 15. In some embodiments, the IL-21Rα mutein comprises two amino acid substitutions compared to SEQ ID NO: 15. In some embodiments, the IL-21Rα mutein comprises three amino acid substitutions compared to SEQ ID NO: 15. In some embodiments, the IL-21Rα mutein comprises four amino acid substitutions compared to SEQ ID NO: 15. In some embodiments, the IL-21Rα mutein comprises five amino acid substitutions compared to SEQ ID NO: 15. In some embodiments, the IL-21Rα mutein comprises more than five amino acid substitutions compared to SEQ ID NO: 15. In some embodiments, the IL-21Rα mutein comprises one to five amino acid substitutions compared to SEQ ID NO: 15.

In some embodiments, the one or more amino acid substitutions are at one or more amino acid positions selected from Y10, Q35, Y36, E38, L39, F67, H68, M70, A71, D72, D73, I74, L94, P126, Y129, M130, K134, S189, S190, and Y191 of the wild-type IL-21Rα sequence. In some embodiments, the IL-21Rα mutein comprises an amino acid substitution at one amino acid position selected from Y10, Q35, Y36, E38, L39, F67, H68, M70, A71, D72, D73, I74, L94, P126, Y129, M130, K134, S189, S190, and Y191 of the wild-type IL-21Rα sequence.

In some embodiments, the one or more amino acid substitutions are at one or more amino acid positions selected from Y36, E38, L39, M70, A71, D72, D73, I74, and L94 of the wild-type IL-21Rα sequence. In some embodiments, the IL-21Rα mutein comprises an amino acid substitution at one amino acid position selected from Y36, E38, L39, M70, A71, D72, D73, I74, and L94 of the wild-type IL-21Rα sequence.

In some embodiments, the IL-21 Rα mutein comprises a sequence different from the wild-type IL-21Rα sequence only at one or more amino acid positions selected from Y10, Q35, Y36, E38, L39, F67, H68, M70, A71, D72, D73, I74, L94, P126, Y129, M130, K134, S189, S190, and Y191 of the wild-type IL-21Rα sequence. In some embodiments, the IL-21 Rα mutein comprises a sequence different from the wild-type IL-21Rα sequence only at one or more amino acid positions selected from Y36, E38, L39, M70, A71, D72, D73, I74, and L94.

In some embodiments, the IL-21 Rα mutein comprises a sequence different from the wild-type IL-21Rα sequence only at one amino acid position selected from Y10, Q35, Y36, E38, L39, F67, H68, M70, A71, D72, D73, I74, L94, P126, Y129, M130, K134, S189, S190, and Y191 of the wild-type IL-21Rα sequence. In some embodiments, the IL-21 Rα mutein comprises a sequence different from the wild-type IL-21Rα sequence only at one amino acid position selected from Y36, E38, L39, M70, A71, D72, D73, I74, and L94.

In some embodiments, the one or more amino acid substitutions are selected from:
a. Y10A
b. Q35K, Q35R, or Q35Y;
c. Y36A, Y36C, Y36E, Y36G, Y36H, Y36I, Y36K, Y36M, Y36N, Y36P, Y36Q, Y36R, Y36S, Y36T, or Y36V;
d. E38A, E38C, E38K, E38R, or E38Y;
e. L39A, L39C, L39E, L39F, L39H, L39K, L39R, L39W, or L39Y;
f. F67A;
g. H68A;
h. M70C, M70D, M70F, M70G, M70H, M70K, M70L, M70N, M70Q, M70R, M70S, M70T, M70V, M70W, or M70Y;
i. A71E, A71F, A71I, A71L, A71Q, A71R, A71W, or A71Y;
j. D72A, D72C, D72E, D72F, D72G, D72H, D72I, D72K, D72L, D72M, D72Q, D72R, D72W, or D72Y;
k. D73C, D73A, D73E, D73H, D73K, D73R, D73W, or D73Y;
l. I74A, I74H, I74K, I74R, or I74W;
m. L94A, L94F, L94K, L94Q, L94R, or L94Y;
n. P126A;
o. Y129A;
p. M130A;
q. K134A;
r. S189A;
s. S190A; and
t. Y191A.

In some embodiments, the one or more amino acid substitutions are selected from:
a. Y36C, Y36E, Y36G, Y36H, Y36I, Y36K, Y36M, Y36N, Y36P, Y36Q, Y36R, Y36S, Y36T, or Y36V;
b. E38C, E38R, or E38Y;
c. L39A, L39C, L39E, L39F, L39H, L39K, L39R, L39W, or L39Y;
d. M70C, M70D, M70F, M70G, M70H, M70K, M70L, M70N, M70Q, M70R, M70S, M70T, M70V, M70W, or M70Y;
e. A71E, A71F, A71I, A71L, A71Q, A71R, A71W, or A71Y;
f. D72A, D72C, D72E, D72F, D72G, D72H, D72I, D72K, D72L, D72M, D72Q, D72R, D72W, or D72Y;
g. D73A
h. I74R, or I74W; and
i. L94A, L94F, L94K, L94Q, L94R, or L94Y.

In some embodiments, the IL-21Rα mutein comprises a sequence selected from SEQ ID NOs: 18-99 and 155-169. In some embodiments, the IL-21Rα mutein comprises a functional fragment of a protein having a sequence selected from SEQ ID NOs: 18-99 and 155-169. The functional fragment can bind to the IL-21 domain.

6.5. Immunocytokines

In another aspect, the present disclosure provides an immunocytokine comprising: (i) an antigen binding protein (ABP) specific to a target protein; (ii) an IL-21 domain; and (iii) an IL-21Rα mutein, wherein the IL-21Rα mutein has a reduced binding affinity to the IL-21 domain compared to a wild-type IL-21Rα.

The immunocytokine can comprise an IL-21Rα mutein disclosed in section 6.4. In some embodiments, the immunocytokine is one selected from R-kine-1 to 66.

6.5.1. Antigen Binding Protein (ABP)

The immunocytokine disclosed herein comprises an antigen binding protein (ABP) specific to a target protein.

The target protein can be a surface protein of an immune cell. In some embodiments, the target protein is a surface protein specific to a T cell. In some embodiments, the target protein is specific to CD4+ or CD8 T+ cells.

In some embodiments, the target protein is an immune checkpoint molecule. In some embodiments, the target protein is PD-1, PD-L1, TIGIT, LAG-3, CTLA-4, TIM-3, CD39, CD38, CD73, CD36, CD25, CD47, CD24, CD20, SIPRα, CD40, or CD20.

In some embodiments, the ABP is an antibody against the target protein or a fragment thereof.

In some embodiments, the ABP is an immune check point inhibitor. In some embodiments, the ABP is anti-PD-1 antibody. In some embodiments, the anti-PD-1 antibody is IgG. In some embodiments, the ABP is anti-CTLA-4 antibody. In some embodiments, the anti-CTLA-4 antibody is IgG. In some embodiments, the ABP is anti-TIGIT antibody. In some embodiments, the anti-TIGIT antibody is IgG. In some embodiments, the ABP is anti-LAG-3 antibody. In some embodiments, the anti-LAG-3 antibody is IgG.

In some embodiments, the ABP comprises Fc fragment selected from a human IgG1 Fc fragment, a human IgG2 Fc fragment, a human IgG3 Fc fragment, and a human IgG4 Fc fragment. In some embodiments, the Fc fragment is a human IgG4 Fc fragment. In some embodiments, the Fc fragment is a human IgG1 Fc fragment. In some embodiments, the Fc fragment comprises a modification for knob-hole interaction. In some embodiments, the Fc fragment is engineered to introduce mutations to reduce effector function of immunoglobulin, which minimize ADCC by reducing the binding affinity for FcγR. In some embodiments, the Fc fragment comprises the sequence selected from SEQ ID NOs: 16, 185-190. In some embodiments, the Fc fragment is engineered to increase stability of the Fc fragment or the immunocytokine containing the Fc fragment. For example, the Fc fragment is engineered to remove Lys (K) at the C-terminal end.

In some embodiments, the ABP is selected from nivolumab, pembrolizumab, cemiplimab, atezolizumab, dostarlimab, durvalumab, avelumab, ipilimumab, tremelimumab, tiragolumab, relatlimab, or a functional variant thereof. A functional variant refers to an ABP having one or more modification compared to an original ABP but maintaining the binding affinity and/or specificity of the original ABP. In some embodiments, the functional variant comprises a binding domain of the original ABP and a heterologous Fc fragment.

In some embodiments, the ABP comprises V_{H} CDR1, V_{H} CDR2, V_{H} CDR3, V_{L} CDR1, V_{L} CDR2, and V_{L} CDR3 sequences of an antibody selected from nivolumab, pembrolizumab, cemiplimab, atezolizumab, dostarlimab, durvalumab, avelumab, ipilimumab, tiragolumab, relatlimab, tremelimumab. In some embodiments, the ABP comprises a heavy chain variable domain of an antibody selected from nivolumab, pembrolizumab, cemiplimab, atezolizumab, dostarlimab, durvalumab, avelumab, ipilimumab, tiragolumab, relatlimab, tremelimumab. In some embodiments, the ABP comprises a light chain variable domain of an antibody selected from nivolumab, pembrolizumab, cemiplimab, atezolizumab, dostarlimab, durvalumab, avelumab, ipilimumab, tiragolumab, relatlimab, tremelimumab. In some embodiments, the ABP comprises a heavy chain variable domain and a light chain variable domain of an antibody selected from nivolumab, pembrolizumab, cemiplimab, atezolizumab, dostarlimab, durvalumab, avelumab, ipilimumab, tiragolumab, relatlimab, tremelimumab.

In some embodiments, the ABP comprises:
a. a heavy chain having the sequence of SEQ ID NO: 1 and a light chain having the sequence of SEQ ID NO: 2;
b. a heavy chain having the sequence of SEQ ID NO: 3 and a light chain having the sequence of SEQ ID NO: 4;
c. a heavy chain having the sequence of SEQ ID NO: 5 and a light chain having the sequence of SEQ ID NO: 6;
d. a heavy chain having the sequence of SEQ ID NO: 7 and a light chain having the sequence of SEQ ID NO: 8;
e. a heavy chain having the sequence of SEQ ID NO: 9 and a light chain having the sequence of SEQ ID NO: 10;
f. a heavy chain having the sequence of SEQ ID NO: 11 and a light chain having the sequence of SEQ ID NO: 12;
g. a heavy chain having the sequence of SEQ ID NO: 13 and a light chain having the sequence of SEQ ID NO: 14;
h. a heavy chain having the sequence of SEQ ID NO: 151 and a light chain having the sequence of SEQ ID NO: 152;
i. a heavy chain having the sequence of SEQ ID NO: 153 and a light chain having the sequence of SEQ ID NO: 154;
j. a heavy chain having the sequence of SEQ ID NO: 225 and a light chain having the sequence of SEQ ID NO: 226; or
k. a heavy chain having the sequence of SEQ ID NO: 227 and a light chain having the sequence of SEQ ID NO: 228.

In some embodiments, the ABP comprises:
a. a heavy chain having the sequence of SEQ ID NO: 1 or a variation thereof, and a light chain having the sequence of SEQ ID NO: 2, wherein the variation comprises a knob-and-hole mutation and/or removal of Lys (K) at the C-terminal end in SEQ ID NO: 1;
b. a heavy chain having the sequence of SEQ ID NO: 3 or a variant thereof, and a light chain having the sequence of SEQ ID NO: 4, wherein the variation comprises a knob-and-hole mutation and/or removal of Lys (K) at the C-terminal end in SEQ ID NO: 3;
c. a heavy chain having the sequence of SEQ ID NO: 5 or a variant thereof, and a light chain having the sequence of SEQ ID NO: 6, wherein the variation comprises a knob-and-hole mutation and/or removal of Lys (K) at the C-terminal end in SEQ ID NO: 5;
d. a heavy chain having the sequence of SEQ ID NO: 7 or a variant thereof, and a light chain having the sequence of SEQ ID NO: 8, wherein the variation comprises a knob-and-hole mutation and/or removal of Lys (K) at the C-terminal end in SEQ ID NO: 7;
e. a heavy chain having the sequence of SEQ ID NO: 9 or a variant thereof, and a light chain having the sequence of SEQ ID NO: 10, wherein the variation comprises a knob-and-hole mutation and/or removal of Lys (K) at the C-terminal end in SEQ ID NO: 9;
f. a heavy chain having the sequence of SEQ ID NO: 11 or a variant thereof, and a light chain having the sequence of SEQ ID NO: 12, wherein the variation comprises a knob-and-hole mutation and/or removal of Lys (K) at the C-terminal end in SEQ ID NO: 11;
g. a heavy chain having the sequence of SEQ ID NO: 13 or a variant thereof, and a light chain having the sequence of SEQ ID NO: 14, wherein the variation comprises a knob-and-hole mutation and/or removal of Lys (K) at the C-terminal end in SEQ ID NO: 13;
h. a heavy chain having the sequence of SEQ ID NO: 151 or a variant thereof, and a light chain having the sequence of SEQ ID NO: 152, wherein the variation comprises a knob-and-hole mutation and/or removal of Lys (K) at the C-terminal end in SEQ ID NO: 151;
i. a heavy chain having the sequence of SEQ ID NO: 153 or a variant thereof, and a light chain having the sequence of SEQ ID NO: 154, wherein the variation comprises a knob-and-hole mutation and/or removal of Lys (K) at the C-terminal end in SEQ ID NO: 153;
j. a heavy chain having the sequence of SEQ ID NO: 225 or a variant thereof, and a light chain having the sequence of SEQ ID NO: 226, wherein the variation comprises a knob-and-hole mutation and/or removal of Lys (K) at the C-terminal end in SEQ ID 225; or
k. a heavy chain having the sequence of SEQ ID NO: 227 or a variant thereof, and a light chain having the sequence of SEQ ID NO: 228, wherein the variation comprises a knob-and-hole mutation and/or removal of Lys (K) at the C-terminal end in SEQ ID 227.

In preferred embodiments, the ABP comprises two Fc moieties. In some embodiments, the IL-21Rα mutein is linked to the first of the two Fc moieties, and the IL-21 domain is linked to the second of the two Fc moieties. In some embodiments, the IL-21Rα mutein is linked to the C terminus of the first of the two Fc moieties, and the IL-21 domain is linked to the C terminus of the second of the two Fc moieties. Various methods known in the art can be used to link the IL-21Rα mutein to the first of the two Fc moieties, and the IL-21 domain to the second of the two Fc moieties. In some embodiments, the IL-21 domain and the IL-21Rα mutein are respectively linked through a non-cleavable peptide linker or without a peptide linker. In some embodiments, the non-cleavable peptide linker is G4S linker having the sequence of SEQ ID NO: 17. In some embodiments, a non-peptide linker is used. In some embodiments, the non-cleavable peptide linker has a sequence selected from SEQ ID NOs: 212-224.

In some embodiments, the ABP comprises an Fc moiety of a human IgG1, IgG2, IgG3 or IgG4. In some embodiments, the Fc moiety comprises any one sequence selected from SEQ ID NOs: 16, and 185-190. In some embodiments, the Fc moiety comprises an C_{H}3 domain of a human IgG1, IgG2, IgG3 or IgG4.

In some embodiments, the ABP comprises an antibody fragment. In some embodiments, the ABP is a Fv fragment, a Fab fragment, a F(ab′)₂ fragment, a Fab′ fragment, a scFv (sFv) fragment, and a scFv-Fc fragment.

In some embodiments, the ABP comprises a knob variant and a hole variant of Fc fragment.

6.5.2. IL-21 Domain

In some embodiments, the IL-21 domain is a human IL-21. In some embodiments, the IL-21 domain is a functional fragment of human IL-21, which can bind to IL-21Rα and activate the target cell. In some embodiments, the IL-21 domain is a functional variant or a homolog of human IL-21, which can bind to IL-21Rα and activate the target cell.

In some embodiments, the IL-21 domain has the sequence of SEQ ID NO: 100 (human IL-21). In some embodiments, the IL-21 domain has a sequence at least 90%, 95%, 97%, 98%, or 99% identical to SEQ ID NO: 100.

6.5.3. Immunocytokine Structure

In some embodiments, the immunocytokine comprises four polypeptide chains-two identical light chains and two heavy chains, joined to form a heterodimer by knobs-into-holes (KiH) interaction. In some embodiments, one of the two heavy chains (“first chain”) is fused to a capping moiety (e.g., IL-21Rα mutein) and the other one (“second chain”) is fused to IL-21. In some embodiments, IL-21 and the capping moiety are fused to the heavy chains through a peptide linker. In some embodiments, the peptide linker is a non-cleavable and flexible peptide linker.

In some embodiments, the first chain comprising from the N terminus to C terminus:
a. a first Fc moiety of the ABP; and
b. an IL-21Rα mutein.

In some embodiments, the first chain further comprises a linker between the first Fc moiety of the ABP and the IL-21Rα mutein.

In some embodiments, the first Fc moiety is a human IgG1, IgG2, IgG3 or IgG4 having any one sequence selected from SEQ ID NOs: 16, 185-190. In some embodiments, the first Fc moiety comprises an C_{H}3 domain of a human IgG1, IgG2, IgG3 or IgG4.

In some embodiments, the first chain comprising from the N terminus to C terminus:
a. a first heavy chain of the ABP; and
b. an IL-21Rα mutein.

In some embodiments, the first chain further comprises a linker between the first heavy chain of the ABP and the IL-21Rα mutein.

In some embodiments, the first heavy chain of the ABP comprises a sequence selected from SEQ ID NOs: 1, 3, 5, 7, 9, 11, 13, 151, 153, 225 and 227 or a variation thereof. In some embodiments, the variation comprises a knob-and-hole mutation in a sequence selected from SEQ ID NOs: 1, 3, 5, 7, 9, 11, 13, 151, 153, 225 and 227. In some embodiments, the variation comprises removal of Lys (K) at the C-terminal end in a sequence selected from SEQ ID NOs: 1, 3, 5, 7, 9, 11, 13, 151, 153, 225 and 227. In some embodiments, the variation comprises a knob-and-hole mutation and removal of Lys (K) at the C-terminal end in a sequence selected from SEQ ID NOs: 1, 3, 5, 7, 9, 11, 13, 151, 153, 225 and 227. The knob-and-hole mutation can be a mutation for making a knob variant or for making a hole variant for knob-and-hole interaction. In some embodiments, the first heavy chain of the ABP comprises the sequence of SEQ ID NO: 103.

In some embodiments, the IL-21Rα mutein comprises a sequence selected from SEQ ID NOs: 18-99 and 155-169.

In some embodiments, the first chain comprises a sequence selected from SEQ ID NOs: 104-150 and 192-209.

In some embodiments, the first chain comprises a sequence selected from SEQ ID NOs: 170-184.

In some embodiments, the second chain comprises from the N terminus to C terminus:
a. a second Fc moiety of the ABP; and
b. an IL-21 domain.

In some embodiments, the second chain further comprises a linker between the second Fc moiety of the ABP and the IL-21 domain.

In some embodiments, the second Fc moiety is a second heavy chain of the ABP.

In some embodiments, the immunocytokine comprises a first heavy chain and a second heavy chain of the ABP. In some embodiments, the first heavy chain comprises a knob mutation and the second heavy chain comprises a hole mutation for knob-and-hole interaction. In some embodiments, the first heavy chain comprises a hole mutation and the second heavy chain comprises a knob mutation for knob-and-hole interaction.

In some embodiments, the second chain has the sequence of SEQ ID NO: 101.

In some embodiments, the immunocytokine comprises two identical light chains. In some embodiments, the light chain has the sequence of SEQ ID NO: 102. In some embodiments, the light chain is the light chain of any one of the ABP is selected from nivolumab, pembrolizumab, cemiplimab, atezolizumab, dostarlimab, durvalumab, avelumab, ipilimumab, tremelimumab, tiragolumab, relatlimab, or a functional variant thereof.

6.6. Polynucleotide, Vector and Host Cells

One aspect of the present disclosure provides one or more polynucleotides encoding the immunocytokine. In some embodiments, the one or more polynucleotides comprise:
a. a first polynucleotide segment encoding a first chain comprising the heavy chain of the ABP and the IL-21Rα mutein;
b. a second polynucleotide segment encoding a second chain comprising the heavy chain of the ABP and the IL-21 domain; and
c. a third polynucleotide segment encoding the light chain of the ABP.

In some embodiments, the first polynucleotide segment comprises a coding sequence of a first chain comprising the heavy chain of the ABP, a peptide linker and the IL-21Rα mutein. In some embodiments, the first polynucleotide segment comprises a coding sequence of a polypeptide having a sequence selected from SEQ ID NOs: 104-150 and 192-209.

In some embodiments, the second polynucleotide segment comprises a coding sequence of a second chain comprising the heavy chain of the ABP, a peptide linker and the IL-21 domain. In some embodiments, the second polynucleotide segment comprises a coding sequence of a polypeptide having the sequence of SEQ ID NO: 101.

In some embodiments, the third polynucleotide segment comprises a coding sequence of a light chain having the sequence of SEQ ID NO: 102.

In some embodiments, the first polynucleotide segment comprises a sequence having at least 85%, 90%, 95%, 96%, 97%, 98%, 98% or 99% identity to SEQ ID NO: 210. In some embodiments, the first polynucleotide comprises a sequence of SEQ ID NO: 210 with one or more nucleotide differences corresponding to the one or more amino acid substitutions in IL21RαMutein.

In some embodiments, the first polynucleotide segment comprises a sequence having at least 85%, 90%, 95%, 96%, 97%, 98%, 98% or 99% identity to SEQ ID NO: 211. In some embodiments, the first polynucleotide comprises a sequence of SEQ ID NO: 211 with one or more nucleotide differences corresponding to the one or more amino acid substitutions in IL21RαMutein.

In some embodiments, the one or more polynucleotides have a sequence which has been codon optimized for expression in a mammalian cell. In some embodiments, the one or more polynucleotides have a sequence which has been codon optimized for expression in a human cell.

In some embodiments, the first polynucleotide segment, the second polynucleotide segment, and the third polynucleotide segment are in a single polynucleotide molecule. In some embodiments, the first polynucleotide segment, the second polynucleotide segment, and the third polynucleotide segment are in multiple polynucleotide molecules.

When more than one polynucleotide segments are present in a single polynucleotide molecule, the multiple polynucleotide segments can be separated by internal ribosome entry site (IRES). In some embodiments, the multiple polynucleotide segments are separated by a self-cleavage site.

In some embodiments, the one or more polynucleotides further comprise a regulatory sequence operably linked to the first, second, or third polynucleotide segment. In some embodiments, the one or more polynucleotides comprise more than one regulatory sequences. In some embodiments, the one or more polynucleotides comprise a regulatory sequence for each of the first, second and third polynucleotide segment.

In some embodiments, the first polynucleotide segment, the second polynucleotide segment, and the third polynucleotide segment are individually present in separate polynucleotide molecules.

In another aspect, the present disclosure provides one or more vectors comprising the one or more polynucleotides. In some embodiments, the first polynucleotide segment, the second polynucleotide segment, and the third polynucleotide segment are individually present in separate vectors. In some embodiments, two or more of the polynucleotide segments are cloned in a single vector.

In some embodiments, the vector is a viral vector. In some embodiments, the vector is an AAV vector or a lentiviral vector. In some embodiments, the vector is non-viral. In some embodiments, the vector is a plasmid.

In some embodiments, the one or more polynucleotides or the one or more vectors are present in a host cell. Accordingly, one aspect of the present disclosure provides a host cell comprising the one or more polynucleotides or the one or more vectors. In some embodiments, the host cell expresses the immunocytokine. In some embodiments, the host cell comprises the immunocytokine. In some embodiments, the host cell releases the immunocytokine. In some embodiments, the host cell is an immune cell. In some embodiments, the host cell is a T cell.

The host cell can be a eukaryotic cell, for example a fungal cell such as yeast. The host cell can be a mammalian cell (which may be a cell in cell culture, or a cell present in a tissue or organ). In some embodiments, the host cell is a human, mouse, rat, rabbit, bovine or dog (or, for example, any other wild, livestock/domesticated animal) cell. In some embodiments, the host cell is a stable cell line cell, or a primary cell, adherent or suspension cell. As examples, the host cell can be a macrophage, osteosarcoma, or CHO, BHK (baby hamster kidney), Bowes human melanoma cell, 911, AT1080, A549, HEK293, or HeLa cell line cell or a mouse primary cell, but not limited thereto. In some embodiments, the host cell is a bacterial cell, such as E. coli.

The eukaryotic cell can be a plant cell (for example a monocotyledonous or dicotyledenous plant cell; typically an experimental, crop and/or ornamental plant cell, for example Arabidopsis, maize); fish (for example Zebra fish; salmon), bird (for example chicken or other domesticated bird), insect (for example Drosophila; bees), Nematoidia or Protista (for example Plasmodium spp or Acantamoeba spp) cell.

In some embodiments, the host cell is used for production of the immunocytokine. In some embodiments, immunocytokine produced from the host cell is purified for therapeutic use. In some embodiments, the host cell is used as therapeutics.

One aspect of the present disclosure provides a polynucleotide encoding the IL-21Rα mutein. In some embodiments, the polynucleotide encoding IL-21Rα mutein having a sequence selected from SEQ ID NOs: 18-99 and 155-169. In some embodiments, the polynucleotide is a viral or non-viral vector. In some embodiments, the polynucleotide further comprises a regulatory sequence operable linked to the coding sequence of IL-21Rα mutein. In another aspect, the present disclosure provides a host cell comprising the polynucleotide encoding the IL-21Rα mutein.

6.7. Method of Treatment

In another aspect, the present disclosure provides a method of administering the immunocytokine or the host cell expressing immunocytokine described above to a subject. In some embodiments, the subject is a cancer patient.

In some embodiments, the administration is effective in enhancing immune response in the subject. In some embodiments, the administration is effective in treating cancer. In some embodiments, the administration is effective in selectively activating an IL-21Rα on a target cell. In some embodiments, the target cell is an immune cell. In some embodiments, the immune cell is a T cell.

In some embodiments, the immunocytokine or the host cell is administered in an amount sufficient to enhance immune response in the subject. In some embodiments, the immunocytokine or the host cell is administered in an amount sufficient to treat cancer. In some embodiments, the immunocytokine or the host cell is administered in an amount sufficient to selectively activate an IL-21Rα on a target cell.

In some embodiments, the method comprises administration of the immunocytokine, the host cell or a pharmaceutical composition comprising the immunocytokine or the host cell.

6.8. Pharmaceutical Composition

In one aspect, the present disclosure provides a pharmaceutical composition comprising the immunocytokine or the host cell comprising the immunocytokine provided herein.

In some embodiments, the pharmaceutical composition comprises the immunocytokine and a pharmaceutically acceptable carrier. In some embodiments, the pharmaceutical composition comprising a host cell expressing the immunocytokine and a pharmaceutically acceptable carrier.

In some embodiments, the pharmaceutically acceptable carrier is a sterile aqueous solution or dispersion and sterile powder for preparation of a sterile injectable solution or dispersion. In some embodiments, the composition is formulated for parenteral injection. The composition can be formulated as a solid, a solution, a microemulsion, a liposome, or other ordered structures suitable to high drug concentration. The carrier can be a solvent or dispersion medium containing, for example, water, ethanol, polyol (e.g., glycerol, propylene glycol and liquid polyethylene glycol), and suitable mixtures thereof. In some cases, the composition contains an isotonic agent, for example, sugar, polyalcohol, for example, sorbitol or sodium chloride.

In some embodiments, the pharmaceutical composition is provided in a unit dose for use as described above.

### 7. EXAMPLES

7.1. Generation of IL21Rα Muteins

Nine (9) amino acid residues of IL-21Rα (M70, A71, D72, D73, Y36, E38, L39, I74, and L94) were predicted to form a binding site to IL-21 based on the predicted structure of IL-21 and IL-21Rα. Some amino acid residues (e.g., Q35) of IL-21Rα were additionally predicted to be involved in the binding affinity from the in-silico analysis (Discovery studio). Their roles in binding to IL-21 were further studied by alanine scanning mutagenesis of each of the amino acid residues of IL-21Rα. IL-21RαMuteins were designed by single amino acid substitution to the 20 amino acid residues in the IL-21Rα amino acid sequence as provided in Table 2.

TABLE 2
WT and IL-21RαMuteins

| No. | Point mutation |
|---|---|
| 1 | WT |
| 2 | Y10A |
| 3 | Q35K |
| 4 | Q35R |
| 5 | Q35Y |
| 6 | Y36A |
| 7 | Y36C |
| 8 | Y36E |
| 9 | Y36G |
| 10 | Y36H |
| 11 | Y36I |
| 12 | Y36K |
| 13 | Y36M |
| 14 | Y36N |
| 15 | Y36P |
| 16 | Y36Q |
| 17 | Y36R |
| 18 | Y36S |
| 19 | Y36T |
| 20 | Y36V |
| 21 | E38A |
| 22 | E38C |
| 23 | E38K |
| 24 | E38R |
| 25 | E38Y |
| 26 | L39A |
| 27 | L39C |
| 28 | L39E |
| 29 | L39F |
| 30 | L39H |
| 31 | L39K |
| 32 | L39R |
| 33 | L39W |
| 34 | L39Y |
| 35 | F67A |
| 36 | H68A |
| 37 | M70C |
| 38 | M70D |
| 39 | M70F |
| 40 | M70G |
| 41 | M70H |
| 42 | M70K |
| 43 | M70L |
| 44 | M70N |
| 45 | M70Q |
| 46 | M70R |
| 47 | M70S |
| 48 | M70T |
| 49 | M70V |
| 50 | M70W |
| 51 | M70Y |
| 52 | A71E |
| 53 | A71F |
| 54 | A71I |
| 55 | A71L |
| 56 | A71Q |
| 57 | A71R |
| 58 | A71W |
| 59 | A71Y |
| 60 | D72A |
| 61 | D72C |
| 62 | D72E |
| 63 | D72G |
| 64 | D72H |
| 65 | D72K |
| 66 | D72Q |
| 67 | D72R |
| 68 | D72W |
| 69 | D72Y |
| 70 | D73A |
| 71 | I74A |
| 72 | I74K |
| 73 | I74R |
| 74 | I74W |
| 75 | L94A |
| 76 | L94F |
| 77 | L94K |
| 78 | L94Q |
| 79 | L94R |
| 80 | L94Y |
| 81 | P126A |
| 82 | Y129A |
| 83 | M130A |
| 84 | K134A |
| 85 | S189A |
| 86 | S190A |
| 87 | Y191A |

The IL21R muteins were generated by introducing one or more point mutations to a plasmid encoding wild type IL-21Rα. Human IgG1Fc (Pro100-Lys330) and IL21R α (Cys20-Glu232) wild type or muteins were conjugated by (G4S)₃ linker. Azurocidin signal peptide was added at the N-terminal for secretion of the expressed protein. After verification of the constructs by sequencing, a large-scale plasmid preparation was performed to obtain enough DNA for transfection.

7.2. SPR Full Kinetics Assay of IL-21Rα Muteins against IL-21

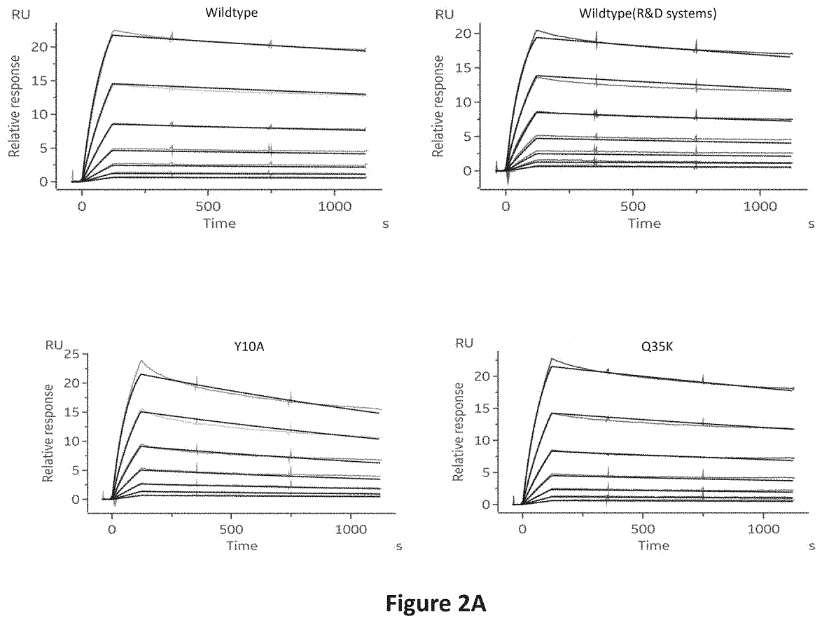

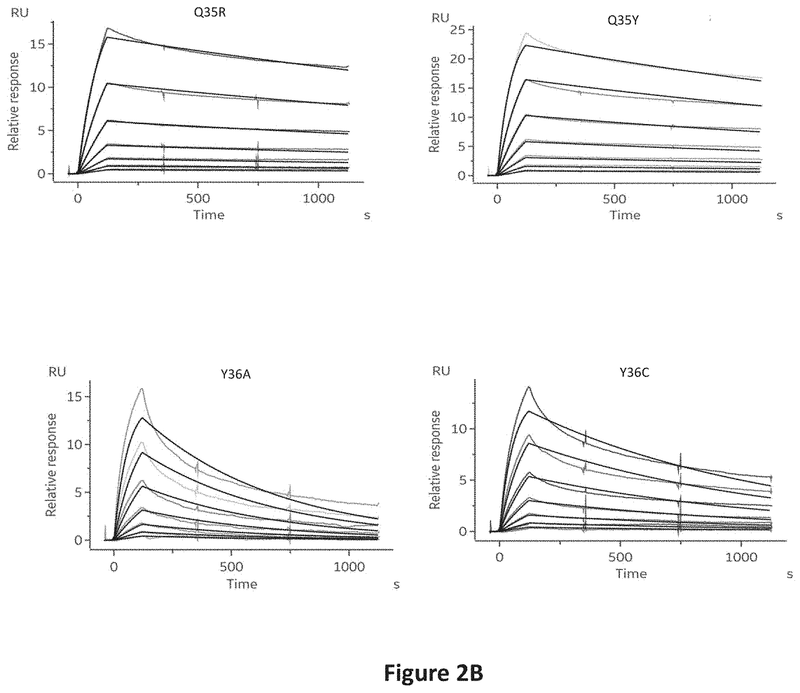

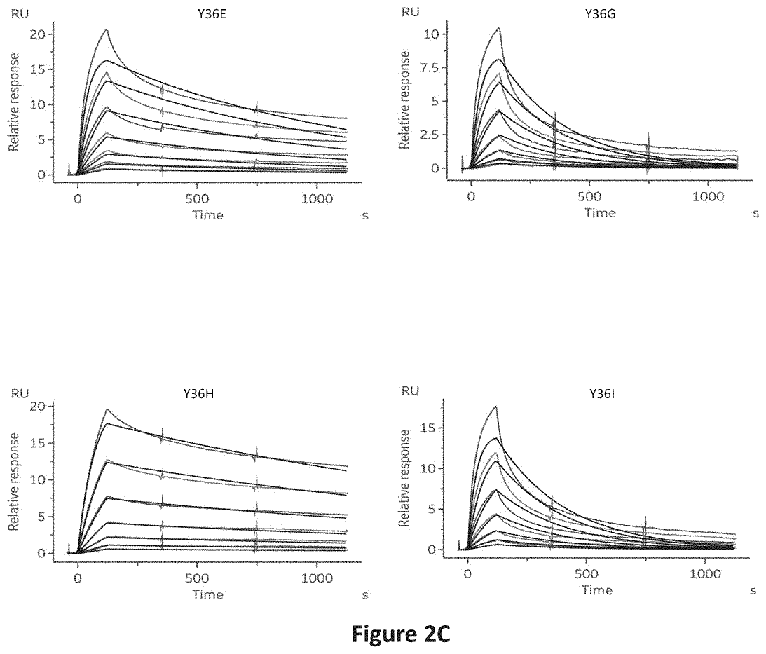

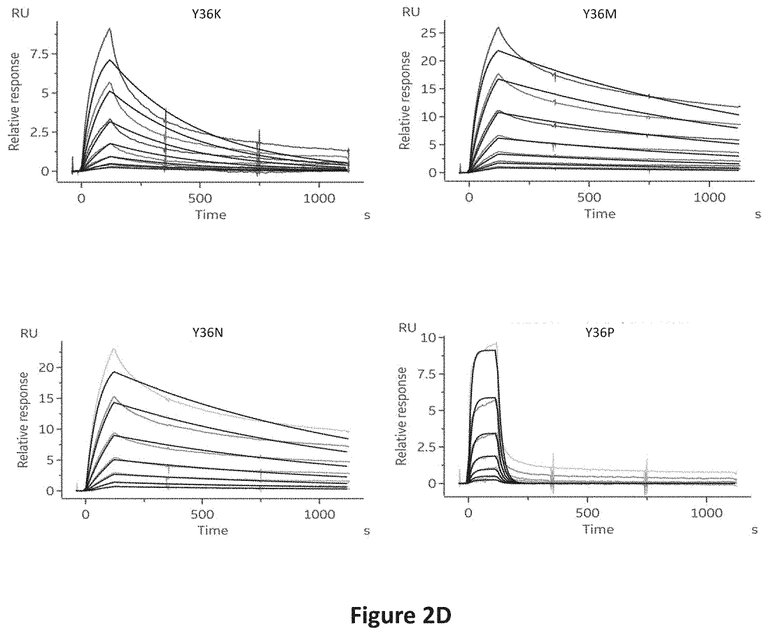

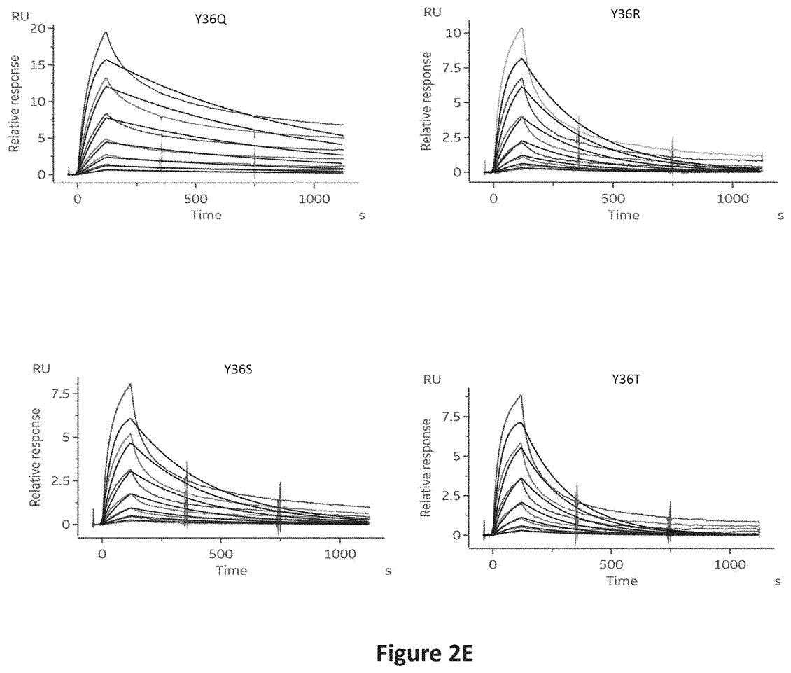

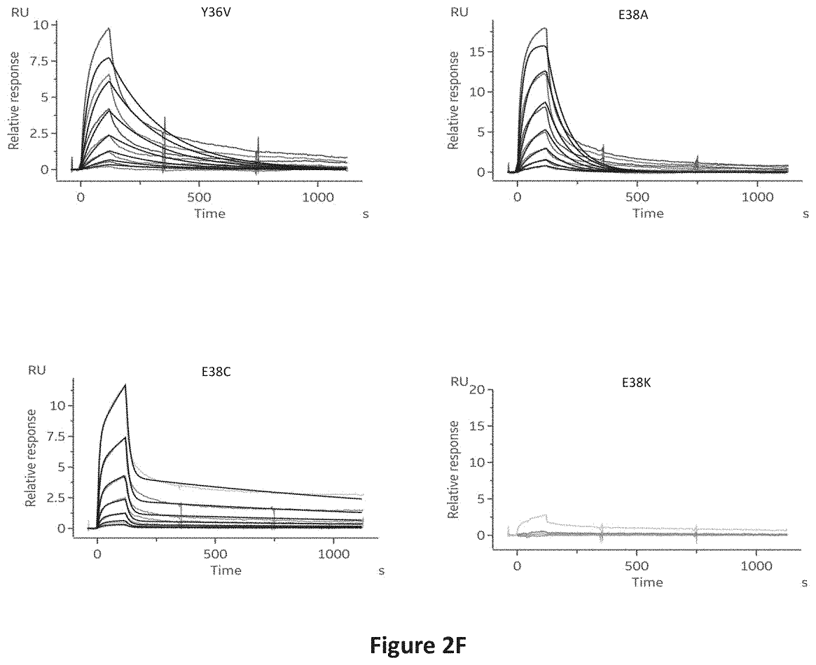

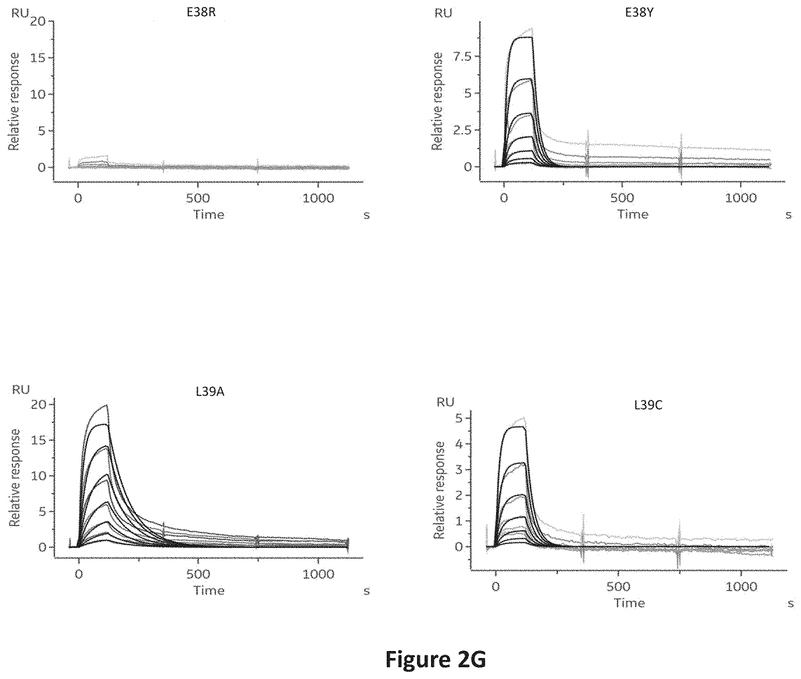

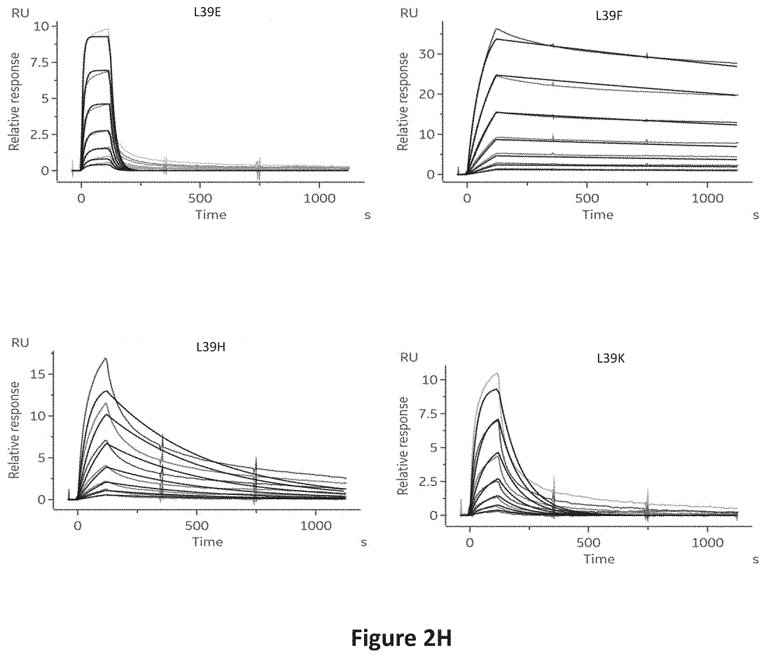

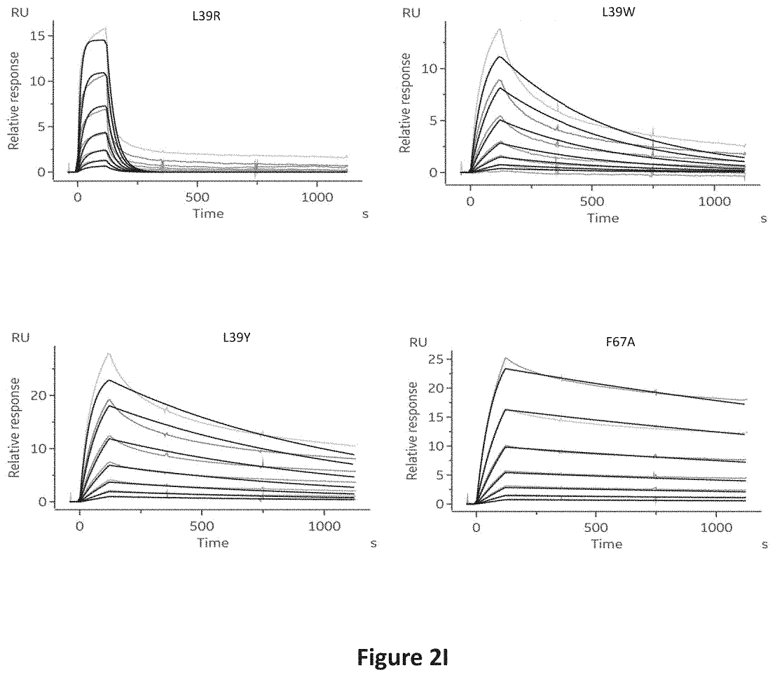

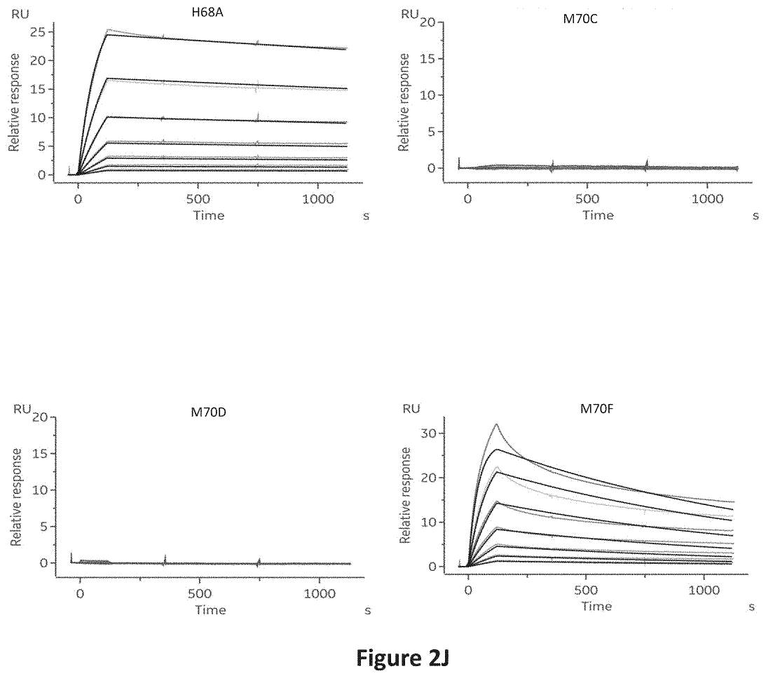

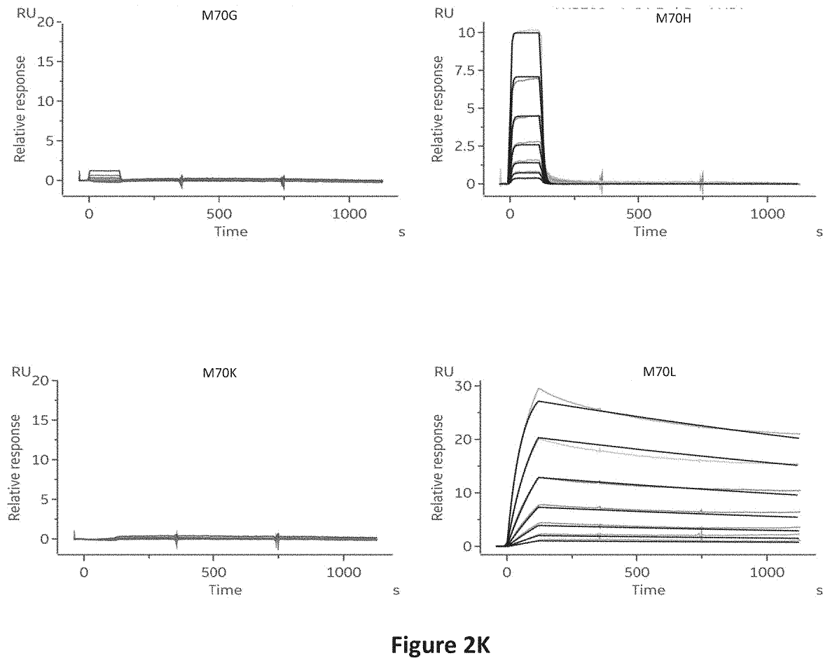

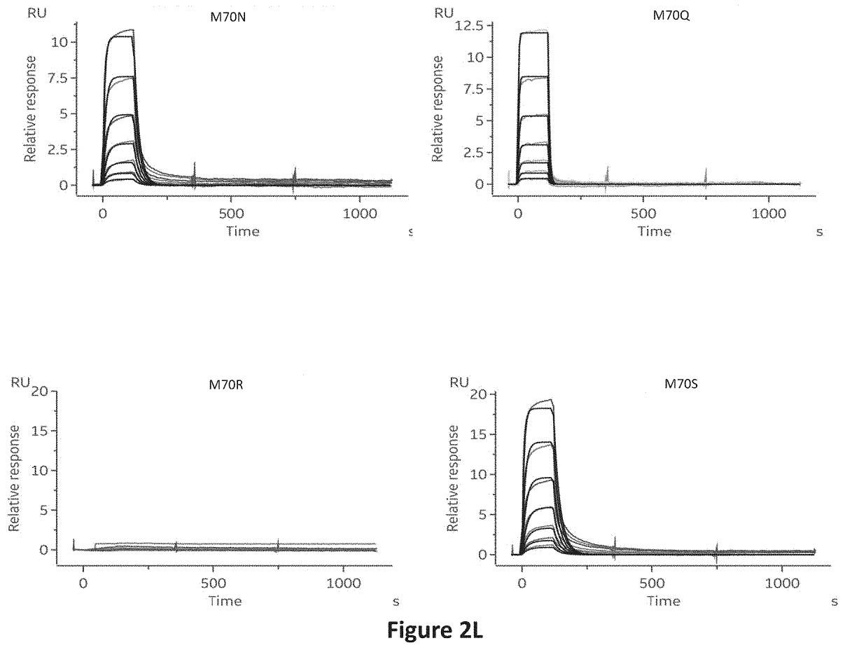

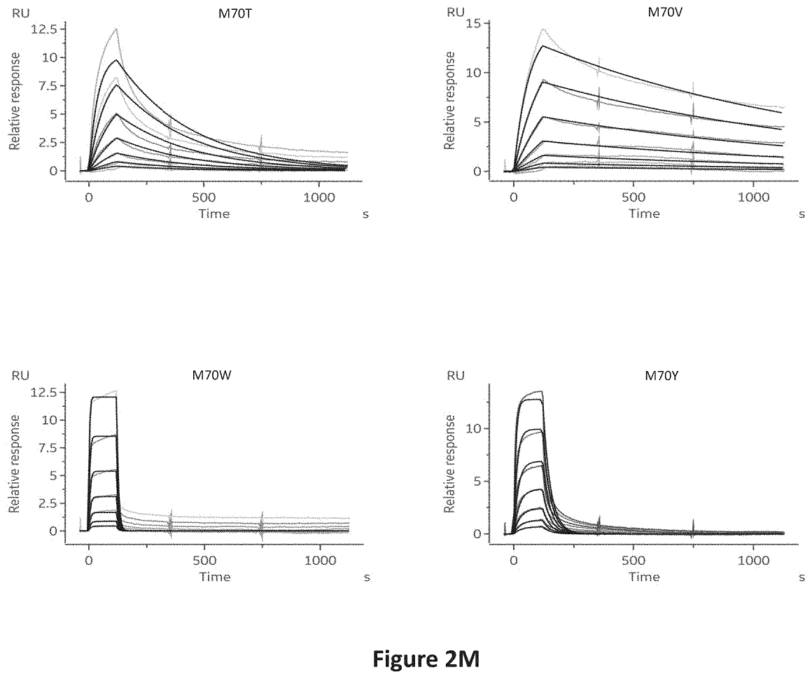

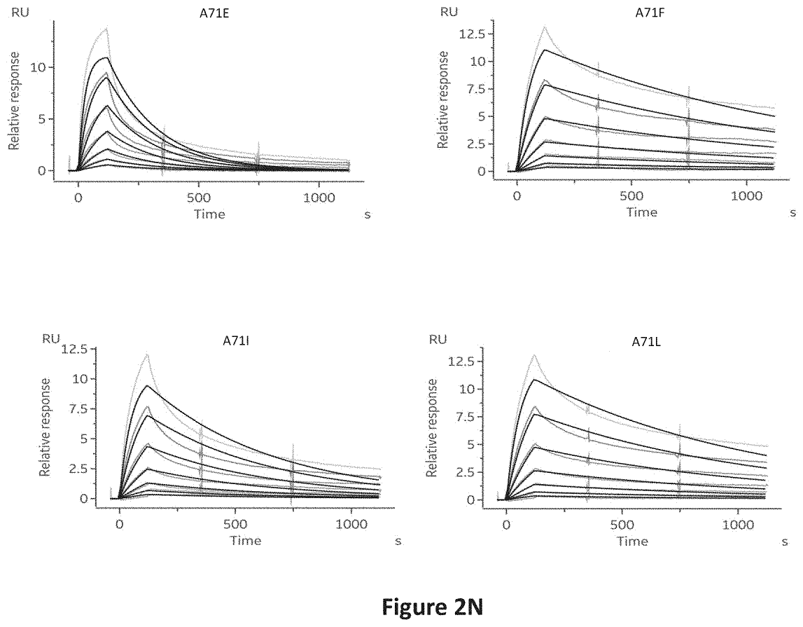

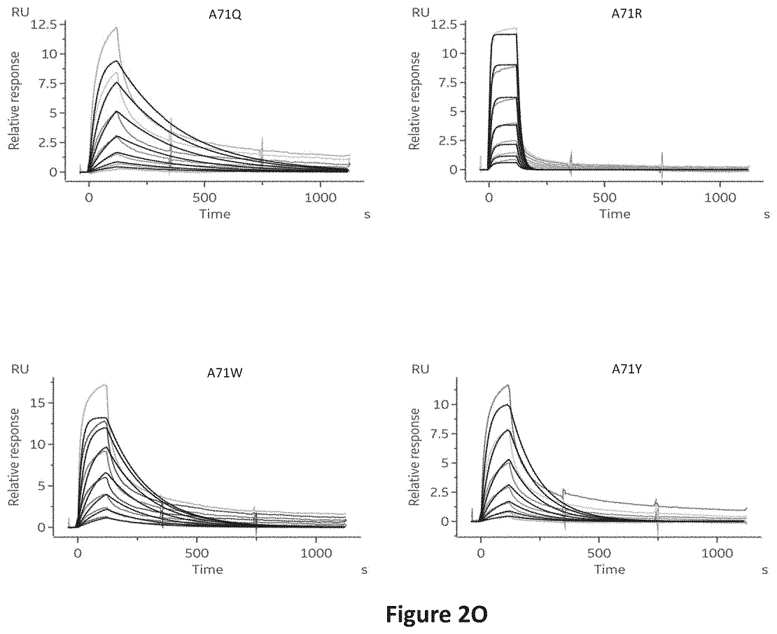

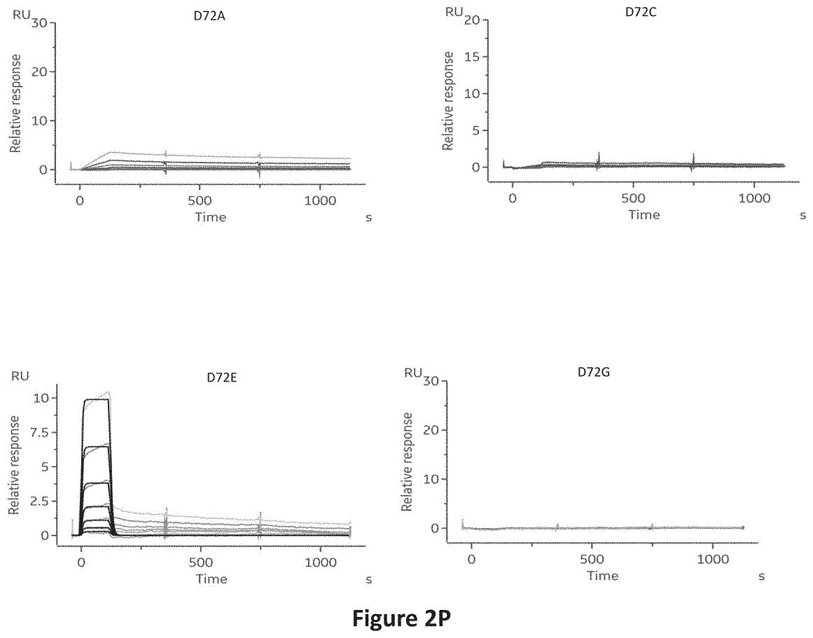

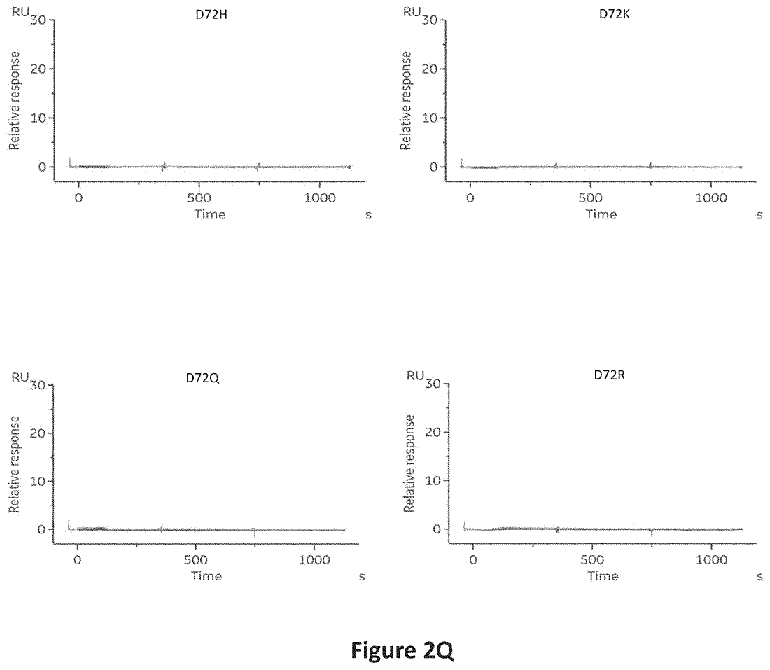

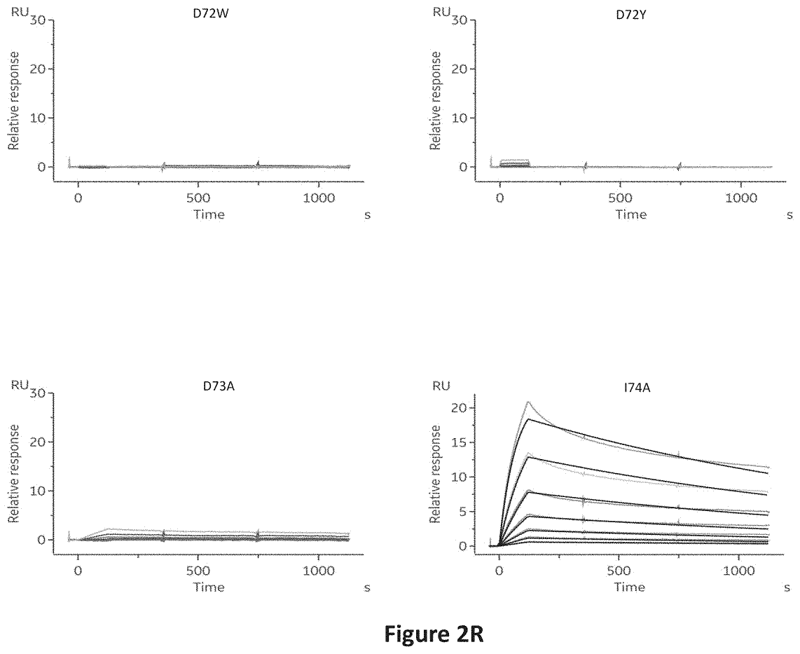

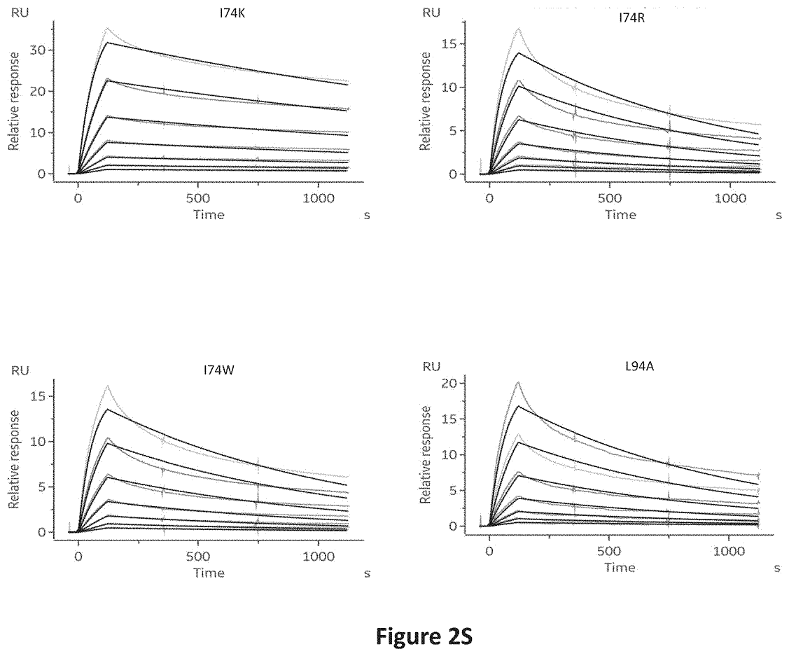

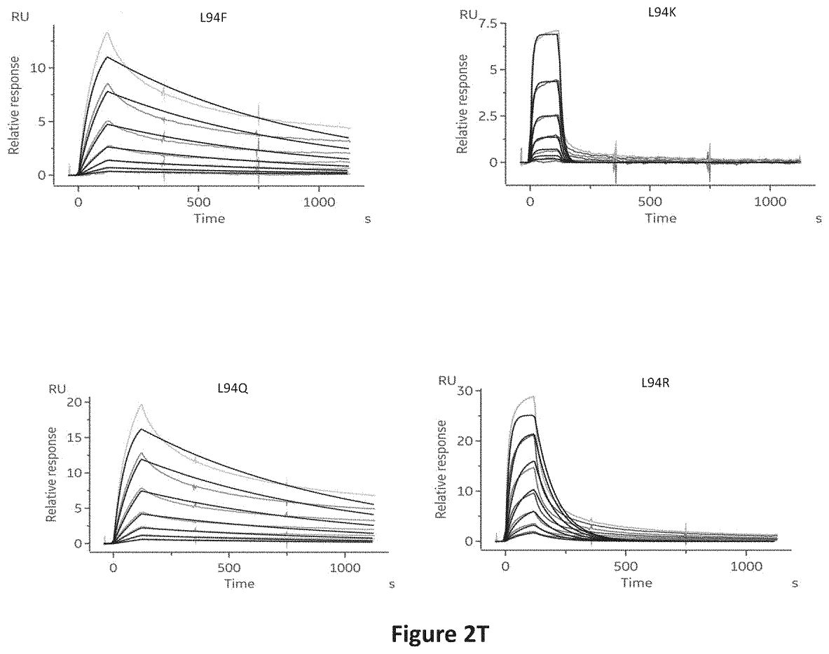

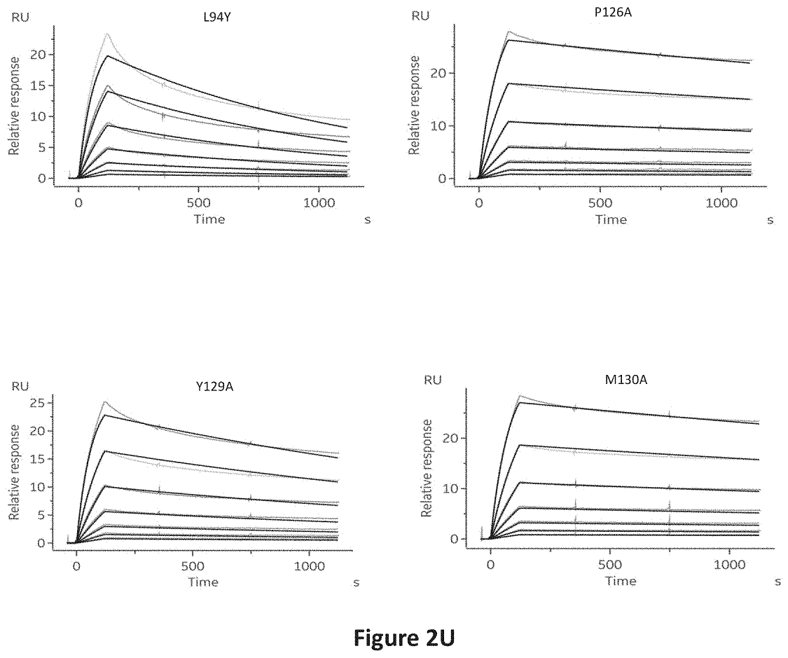

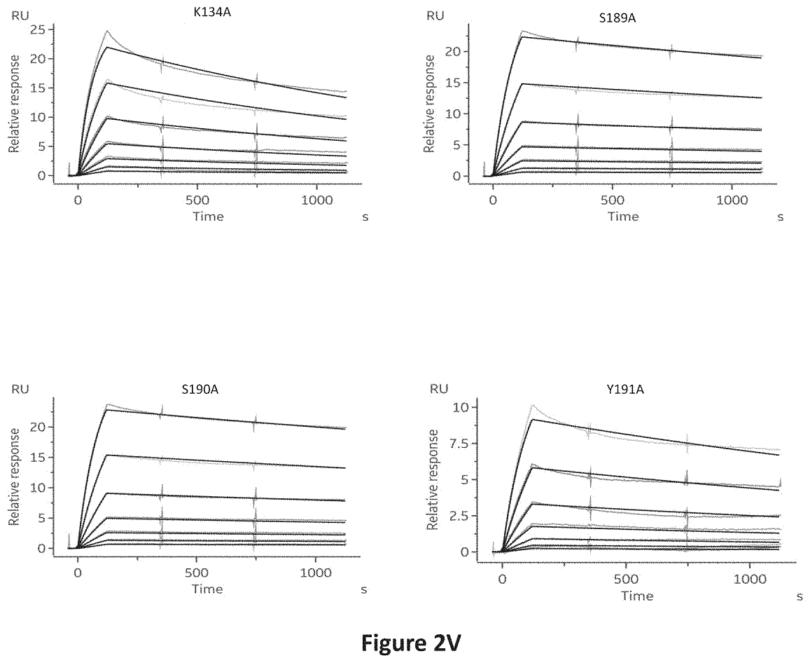

Bivalent Fc-fusion proteins (IgG1) were generated with each of the muteins [IL21RαMutein-Fc] and their binding affinity to IL-21 was measured by SPR (Biacore 8K) (Table 3 and FIGS. 2A-2V). IL21Rα Muteins and IL-21's affinity was tested by CM5 sensor chip. 400 mM EDC and 100 mM NHS (Cytiva) were injected to CMS sensor chip for 420 s with a flow rate of 10 μL/min as activator prior to injecting 1.55 ug/mL of hIL-21 in 10 mM NaAc (pH 5.0) to the channel for 240 s at a flow rate of 10 μL/min. The chip was deactivated by 1M ethanolamine-HCl (Cytiva) at flow rate of 10 μL/min for 420 s.

Multiple cycle kinetics were used to perform the assay. hIL-21R (WT or Muteins) at 7 different concentrations and a running buffer were injected orderly to Fc1-Fc2 at a flow rate of 80 μL/min for an association phase of 120 s, followed by 1000 s dissociation. 10 mM glycine pH1.5 was injected as a regeneration buffer following every dissociation phase.

The sensorgrams from the reference channel Fc1 and the buffer channel were subtracted from the test sensorgrams. The experimental data was fitted by 1:1 binding model or heterogeneous ligand. Molecular weight of 15 kDa were used to calculate the molar concentration of IL-21.

The data from the SPR full kinetics assay of IL-21Rα muteins against IL21 are provided in FIGS. 2A-2V. The binding affinities of muteins were measured using Biacore 8K and provided in Table 3.

TABLE 3
Binding affinity of Fc-IL21Rα (WT
and mutein) against IL21 (1:1 binding model)

| No. | Point Mutation | K_{D} (M) |
|---|---|---|
| 1 | WT | 1.67E−10 |
| 2 | Y10A | 5.23E−10 |
| 3 | Q35K | 3.43E−10 |
| 4 | Q35R | 4.91E−10 |
| 5 | Q35Y | 3.66E−10 |
| 6 | Y36A | 2.13E−09 |
| 7 | Y36C | 1.10E−09 |
| 8 | Y36E | 6.88E−10 |
| 9 | Y36G | 2.76E−09 |
| 10 | Y36H | 6.11E−10 |
| 11 | Y36I | 2.55E−09 |
| 12 | Y36K | 2.97E−09 |
| 13 | Y36M | 7.26E−10 |
| 14 | Y36N | 8.94E−10 |
| 15 | Y36P | 2.46E−07 |
| 16 | Y36Q | 1.04E−09 |
| 17 | Y36R | 6.67E−09 |
| 18 | Y36S | 2.58E−09 |
| 19 | Y36T | 3.98E−09 |
| 20 | Y36V | 3.33E−09 |
| 21 | E38A | 2.85E−08 |
| 22 | E38C | 2.40E−08 |
| 23 | E38K | >1.00E−06* |
| 24 | E38R | >1.00E−06 |
| 25 | E38Y | 1.77E−07 |
| 26 | L39A | 2.36E−08 |
| 27 | L39C | 1.50E−07 |
| 28 | L39E | 1.02E−07 |
| 29 | L39F | 2.65E−10 |
| 30 | L39H | 1.98E−09 |
| 31 | L39K | 1.41E−08 |
| 32 | L39R | 9.70E−08 |
| 33 | L39W | 2.33E−09 |
| 34 | L39Y | 8.16E−10 |
| 35 | F67A | 4.37E−10 |
| 36 | H68A | 1.68E−10 |
| 37 | M70C | >1.00E−06 |
| 38 | M70D | >1.00E−06 |
| 39 | M70F | 1.18E−09 |
| 40 | M70G | >1.00E−06 |
| 41 | M70H | 6.94E−07 |
| 42 | M70K | >1.00E−06 |
| 43 | M70L | 6.42E−10 |
| 44 | M70N | 1.16E−07 |
| 45 | M70Q | 6.85E−07 |
| 46 | M70R | >1.00E−06 |
| 47 | M70S | 8.54E−08 |
| 48 | M70T | 5.12E−09 |
| 49 | M70V | 9.74E−10 |
| 50 | M70W | 7.06E−07 |
| 51 | M70Y | 7.92E−08 |
| 52 | A71E | 2.92E−09 |
| 53 | A71F | 1.01E−09 |
| 54 | A71I | 1.97E−09 |
| 55 | A71L | 1.26E−09 |
| 56 | A71Q | 5.01E−09 |
| 57 | A71R | 4.09E−07 |
| 58 | A71W | 1.85E−08 |
| 59 | A71Y | 1.03E−08 |
| 60 | D72A | >1.00E−06 |
| 61 | D72C | >1.00E−06 |
| 62 | D72E | 1.14E−06 |
| 63 | D72G | >1.00E−06 |
| 64 | D72H | >1.00E−06 |
| 65 | D72K | >1.00E−06 |
| 66 | D72Q | >1.00E−06 |
| 67 | D72R | >1.00E−06 |
| 68 | D72W | >1.00E−06 |
| 69 | D72Y | >1.00E−06 |
| 70 | D73A | >1.00E−06 |
| 71 | I74A | 7.66E−10 |
| 72 | I74K | 5.18E−10 |
| 73 | I74R | 1.30E−09 |
| 74 | I74W | 1.15E−09 |
| 75 | L94A | 1.44E−09 |
| 76 | L94F | 1.49E−09 |
| 77 | L94K | 2.79E−07 |
| 78 | L94Q | 1.19E−09 |
| 79 | L94R | 3.97E−08 |
| 80 | L94Y | 1.13E−09 |
| 81 | P126A | 2.77E−10 |
| 82 | Y129A | 5.15E−10 |
| 83 | M130A | 2.54E−10 |
| 84 | K134A | 6.18E−10 |
| 85 | S189A | 2.90E−10 |
| 86 | S190A | 2.45E−10 |
| 87 | Y191A | 6.67E−10 |

*1.00E−06 is the minimum detection limit.

After the measurement of binding affinities of the muteins [IL21Rα(mut)-Fc] to IL-21, 58 muteins were classified based on the degree of reduction in their binding affinity to IL-21, e.g., 10, 100, and 1000-fold reduction compared to wild-type IL-21Rα. Finally, 66 IgG-fusion proteins were generated in which an IL-21 and one of the muteins of IL21-Rα are fused to one of two heavy chains of IgG, respectively.

7.3. Generation of Immunocytokines (αPD-1IL21RαMutein/IL21)

The immunocytokine described herein, αPD-1IL21RαMutein/IL21, can exhibit an anti-cancer immune response by working as an ICB and inducing signal transduction mediated by the complex of IL21 receptor (IL21Rα/common gamma chain) expressed on the surface of target cells. αPD-1IL21RαMutein/IL21 is designed to primarily activate target immune cells only when it binds to PD-1. It leads competition between IL21RαMutein and endogenous IL21Rα (e.g., IL21RαWT) of target cells by the proximity, inducing stripping of IL21RαMutein from the moiety of IL-21, causing it to bind to endogenous IL21Rα.

Previously, a fusion protein comprising an attenuated IL-21 fused to the c-terminal ends of the anti-PD-1 antibody was developed for treatment of cancer by activation of immune cells. In the fusion protein, an attenuated IL-21 was used to reduce off-target effects and the anti-PD-1 antibody was used to improve bioavailability at the target. The attenuated IL-21 includes two point mutations in the amino acid sequence of IL-21, making its max potency reduced to 70-80% compared to wild-type IL-21 (See Shanling Shen et al. Engineered IL-21 Cytokine Muteins Fused to Anti-PD-1 Antibodies Can Improve CD8+ T Cell Function and Anti-tumor Immunity. Front Immunol. 2020 May 8; 11:832).

Unlike the fusion protein comprising an attenuated IL-21, αPD-1IL21RαMutein/IL21 includes an unmodified IL-21, thus they can have effects on target cells similar to wild-type IL-21. 66 immunocytokines, each containing a different mutein of IL21Rα, were generated. The 66 immunocytokines (Table 4) include one or two amino acid substitutions. More specifically, the immunocytokines (R-kine-1 to 66) includes (i) a first chain comprising a heavy chain, G4S linker and IL-21RαMutein; (ii) a second chain comprising a heavy chain, G4S linker and a human IL-21; and (iii) two light chains, as specified in Table 4.

TABLE 4

| | | Sequence of first | Sequence of second | |
|---|---|---|---|---|
| | IL-21Rα | chain (Heavy | chain (Heavy | |
| | Mutation | Chain-G4S Linker- | Chain-G4S Linker- | Sequence of two |
| No. | Site | IL-21RαMutein) | human IL-21) | light chains |
| R-kine-1 | WT | SEQ ID NO: 191 | SEQ ID NO: 101 | SEQ ID NO: 102 |
| R-kine-2 | Y36C | SEQ ID NO: 104 | SEQ ID NO: 101 | SEQ ID NO: 102 |
| R-kine-3 | Y36E | SEQ ID NO: 105 | SEQ ID NO: 101 | SEQ ID NO: 102 |
| R-kine-4 | Y36G | SEQ ID NO: 106 | SEQ ID NO: 101 | SEQ ID NO: 102 |
| R-kine-5 | Y36H | SEQ ID NO: 107 | SEQ ID NO: 101 | SEQ ID NO: 102 |
| R-kine-6 | Y36I | SEQ ID NO: 108 | SEQ ID NO: 101 | SEQ ID NO: 102 |
| R-kine-7 | Y36K | SEQ ID NO: 109 | SEQ ID NO: 101 | SEQ ID NO: 102 |
| R-kine-8 | Y36M | SEQ ID NO: 110 | SEQ ID NO: 101 | SEQ ID NO: 102 |
| R-kine-9 | Y36N | SEQ ID NO: 111 | SEQ ID NO: 101 | SEQ ID NO: 102 |
| R-kine-10 | Y36P | SEQ ID NO: 112 | SEQ ID NO: 101 | SEQ ID NO: 102 |
| R-kine-11 | Y36Q | SEQ ID NO: 113 | SEQ ID NO: 101 | SEQ ID NO: 102 |
| R-kine-12 | Y36R | SEQ ID NO: 114 | SEQ ID NO: 101 | SEQ ID NO: 102 |
| R-kine-13 | Y36S | SEQ ID NO: 115 | SEQ ID NO: 101 | SEQ ID NO: 102 |
| R-kine-14 | Y36T | SEQ ID NO: 116 | SEQ ID NO: 101 | SEQ ID NO: 102 |
| R-kine-15 | Y36V | SEQ ID NO: 117 | SEQ ID NO: 101 | SEQ ID NO: 102 |
| R-kine-16 | E38C | SEQ ID NO: 118 | SEQ ID NO: 101 | SEQ ID NO: 102 |
| R-kine-17 | E38R | SEQ ID NO: 200 | SEQ ID NO: 101 | SEQ ID NO: 102 |
| R-kine-18 | E38Y | SEQ ID NO: 119 | SEQ ID NO: 101 | SEQ ID NO: 102 |
| R-kine-19 | L39C | SEQ ID NO: 120 | SEQ ID NO: 101 | SEQ ID NO: 102 |
| R-kine-20 | L39E | SEQ ID NO: 121 | SEQ ID NO: 101 | SEQ ID NO: 102 |
| R-kine-21 | L39H | SEQ ID NO: 122 | SEQ ID NO: 101 | SEQ ID NO: 102 |
| R-kine-22 | L39K | SEQ ID NO: 123 | SEQ ID NO: 101 | SEQ ID NO: 102 |
| R-kine-23 | L39R | SEQ ID NO: 124 | SEQ ID NO: 101 | SEQ ID NO: 102 |
| R-kine-24 | L39W | SEQ ID NO: 125 | SEQ ID NO: 101 | SEQ ID NO: 102 |
| R-kine-25 | L39Y | SEQ ID NO: 126 | SEQ ID NO: 101 | SEQ ID NO: 102 |
| R-kine-26 | M70F | SEQ ID NO: 127 | SEQ ID NO: 101 | SEQ ID NO: 102 |
| R-kine-27 | M70H | SEQ ID NO: 128 | SEQ ID NO: 101 | SEQ ID NO: 102 |
| R-kine-28 | M70N | SEQ ID NO: 129 | SEQ ID NO: 101 | SEQ ID NO: 102 |
| R-kine-29 | M70Q | SEQ ID NO: 130 | SEQ ID NO: 101 | SEQ ID NO: 102 |
| R-kine-30 | M70S | SEQ ID NO: 131 | SEQ ID NO: 101 | SEQ ID NO: 102 |
| R-kine-31 | M70T | SEQ ID NO: 132 | SEQ ID NO: 101 | SEQ ID NO: 102 |
| R-kine-32 | M70V | SEQ ID NO: 133 | SEQ ID NO: 101 | SEQ ID NO: 102 |
| R-kine-33 | M70W | SEQ ID NO: 134 | SEQ ID NO: 101 | SEQ ID NO: 102 |
| R-kine-34 | M70Y | SEQ ID NO: 135 | SEQ ID NO: 101 | SEQ ID NO: 102 |
| R-kine-35 | A71E | SEQ ID NO: 136 | SEQ ID NO: 101 | SEQ ID NO: 102 |
| R-kine-36 | A71F | SEQ ID NO: 137 | SEQ ID NO: 101 | SEQ ID NO: 102 |
| R-kine-37 | A71I | SEQ ID NO: 138 | SEQ ID NO: 101 | SEQ ID NO: 102 |
| R-kine-38 | A71L | SEQ ID NO: 139 | SEQ ID NO: 101 | SEQ ID NO: 102 |
| R-kine-39 | A71Q | SEQ ID NO: 140 | SEQ ID NO: 101 | SEQ ID NO: 102 |
| R-kine-40 | A71R | SEQ ID NO: 141 | SEQ ID NO: 101 | SEQ ID NO: 102 |
| R-kine-41 | A71W | SEQ ID NO: 142 | SEQ ID NO: 101 | SEQ ID NO: 102 |
| R-kine-42 | A71Y | SEQ ID NO: 143 | SEQ ID NO: 101 | SEQ ID NO: 102 |
| R-kine-43 | I74R | SEQ ID NO: 144 | SEQ ID NO: 101 | SEQ ID NO: 102 |
| R-kine-44 | I74W | SEQ ID NO: 145 | SEQ ID NO: 101 | SEQ ID NO: 102 |
| R-kine-45 | L94F | SEQ ID NO: 146 | SEQ ID NO: 101 | SEQ ID NO: 102 |
| R-kine-46 | L94K | SEQ ID NO: 147 | SEQ ID NO: 101 | SEQ ID NO: 102 |
| R-kine-47 | L94Q | SEQ ID NO: 148 | SEQ ID NO: 101 | SEQ ID NO: 102 |
| R-kine-48 | L94R | SEQ ID NO: 149 | SEQ ID NO: 101 | SEQ ID NO: 102 |
| R-kine-49 | L94Y | SEQ ID NO: 150 | SEQ ID NO: 101 | SEQ ID NO: 102 |
| R-kine-50 | M70C | SEQ ID NO: 201 | SEQ ID NO: 101 | SEQ ID NO: 102 |
| R-kine-51 | M70D | SEQ ID NO: 202 | SEQ ID NO: 101 | SEQ ID NO: 102 |
| R-kine-52 | M70G | SEQ ID NO: 203 | SEQ ID NO: 101 | SEQ ID NO: 102 |
| R-kine-53 | M70R | SEQ ID NO: 204 | SEQ ID NO: 101 | SEQ ID NO: 102 |
| R-kine-54 | D72A | SEQ ID NO: 205 | SEQ ID NO: 101 | SEQ ID NO: 102 |
| R-kine-55 | D72E | SEQ ID NO: 206 | SEQ ID NO: 101 | SEQ ID NO: 102 |
| R-kine-56 | D72Q | SEQ ID NO: 207 | SEQ ID NO: 101 | SEQ ID NO: 102 |
| R-kine-57 | D72R | SEQ ID NO: 208 | SEQ ID NO: 101 | SEQ ID NO: 102 |
| R-kine-58 | D73A | SEQ ID NO: 209 | SEQ ID NO: 101 | SEQ ID NO: 102 |
| R-kine-59 | Y36A + D72E | SEQ ID NO: 192 | SEQ ID NO: 101 | SEQ ID NO: 102 |
| R-kine-60 | Y36A + L94R | SEQ ID NO: 193 | SEQ ID NO: 101 | SEQ ID NO: 102 |
| R-kine-61 | E38A + D72E | SEQ ID NO: 194 | SEQ ID NO: 101 | SEQ ID NO: 102 |
| R-kine-62 | E38A + L94K | SEQ ID NO: 195 | SEQ ID NO: 101 | SEQ ID NO: 102 |
| R-kine-63 | E38A + L94R | SEQ ID NO: 196 | SEQ ID NO: 101 | SEQ ID NO: 102 |
| R-kine-64 | E38R + D72R | SEQ ID NO: 197 | SEQ ID NO: 101 | SEQ ID NO: 102 |
| R-kine-65 | D72E + L94K | SEQ ID NO: 198 | SEQ ID NO: 101 | SEQ ID NO: 102 |
| R-kine-66 | D72E + L94R | SEQ ID NO: 199 | SEQ ID NO: 101 | SEQ ID NO: 102 |

For production of the immunocytokines, 6.0×10⁶/mL of ExpiCHO cells (ThermoFisher) with higher than 95% viability were prepared in 100 mL of cell culture media. 100 μg of the plasmid DNA encoding the immunocytokine was mixed with the ExpiFectamine™ CHO transfection reagent (ThermoFisher) and the mixture was added to the cell culture media. The cell culture was incubated in a platform shaker with the rotation rate at 150 rpm. The temperature was maintained at 37° C. while CO₂ level at 8%.

After ten days of incubation, the cells were pelleted by centrifuging at 4000 rpm, 25° C. for 10 minutes. Supernatant was collected for purification and gel electrophoresis. The supernatant was loaded on SDS-PAGE gel, following the instruction for NuPAGE™ 4-12% Bis-Tris Protein Gels (ThermoFisher). PageRuler™ Unstained Protein Ladder (ThermoFisher) was used alongside with the protein samples to determine the molecular weight of the protein. Fusion proteins were then purified by Protein A column (Cytiva) followed by SEC column (Cytiva).

7.4. Homogeneous Time-Resolved Fluorescence (HTRF) Phospho-STAT3 Assay of Immunocytokines (αPD-1IL21RαMutein/IL21)

The 66 immunocytokines were evaluated by measuring phosphorylation of STAT3 in HTRF-based high-throughput assay. Human cutaneous T lymphocyte cell lines (H9 (Cobioer), derivative of Hut78 cells) and H9 cells that stably expressing a programmed cell death protein 1 (PD-1(+) H9) were used in the pSTAT3 assay. Cells were grown in IMDM medium (Gibco) containing 20% fetal bovine serum (FBS, Gibco) and 1% penicillin/streptomycin (Sigma Aldrich) for H9. 3 μg/mL puromycin (Invivogen) was additionally added for PD-1 positive H9 cells. Subculture of cells was conducted every 48 hours to avoid high density which could arrest the cell cycle.

Measuring pSTAT3 production was conducted to investigate the activation of cells by IL-21 binding with IL-21 receptors and common gamma chains. The high production of pSTAT3 was considered as a marker of strong reaction of treated materials. To conduct experiments, pSTAT3 ELISA kit (Perkin Elmer, MA) and Flex Station 3 (Molecular Devices, CA) were used following the manufacturer's user guide.

The detailed description is as follows. PD-1(−) H9 or PD-1(+) H9 cells were incubated with serum free media on overnight. After incubation, spin-down cells (with 125 g) were harvested with HBSS (Gibco) solution and seeded on white 96 well low volume plate (Cisbio) by 2.5×10⁴ cells/well/8 μL. Compounds for evaluation were prepared with 3× concentration of final concentration and treated to cells for 30 minutes at 37° C. The lysis buffer was added to the wells for 30 minutes and then reagents for HTRF reaction were treated following the manufacturer's protocol. After 24 hours, the HTRF reaction was measured by Flex Station 3 equipment.

The non-linear analysis (4 parameters logistic regression) was conducted to calculate experiment parameters including EC₅₀, Maximal response, and Hillslope. The Black and Leff operational model was adopted to estimate the compound's intrinsic efficacy.

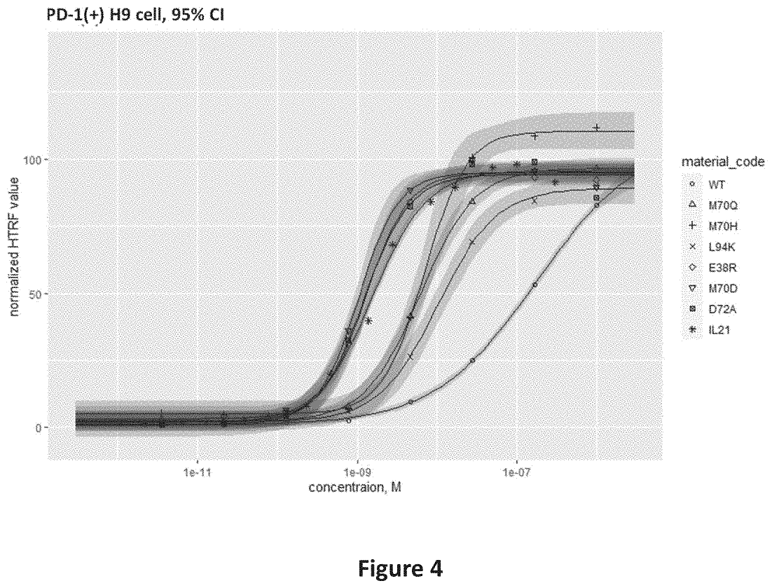

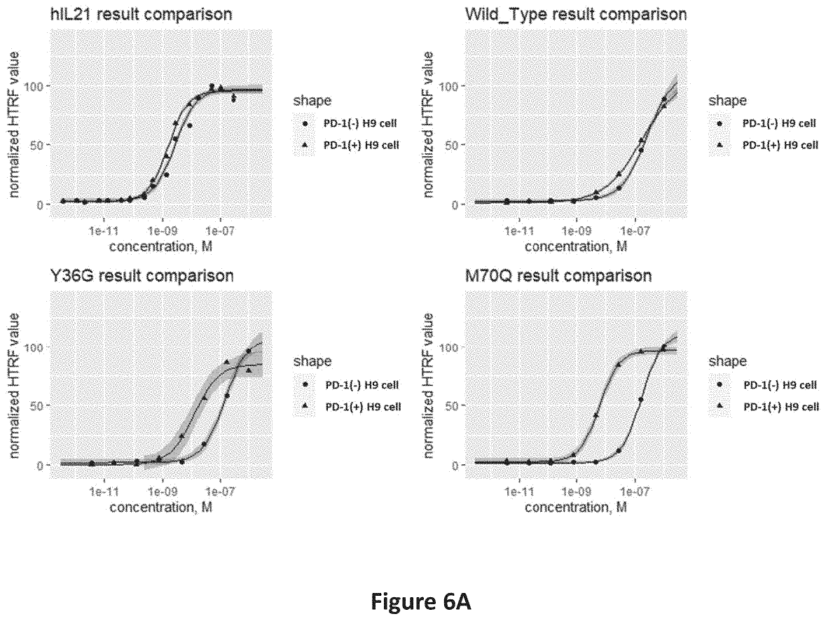

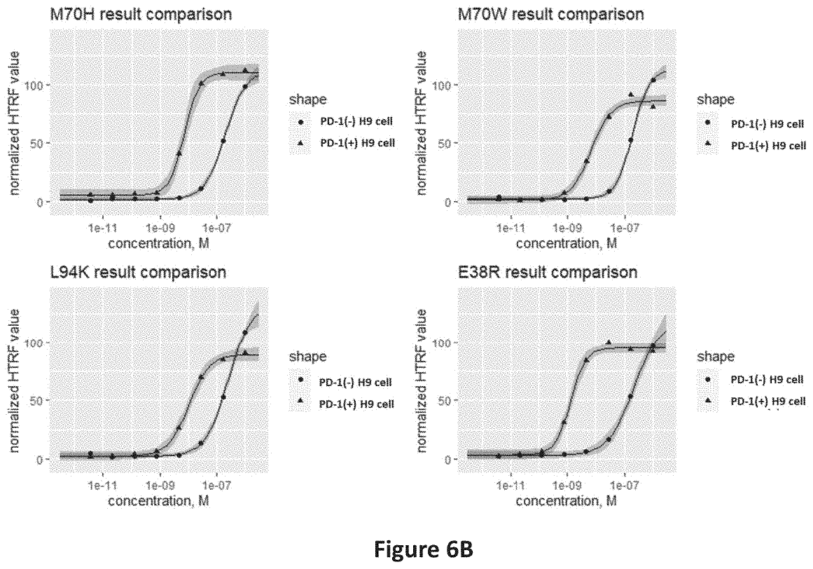

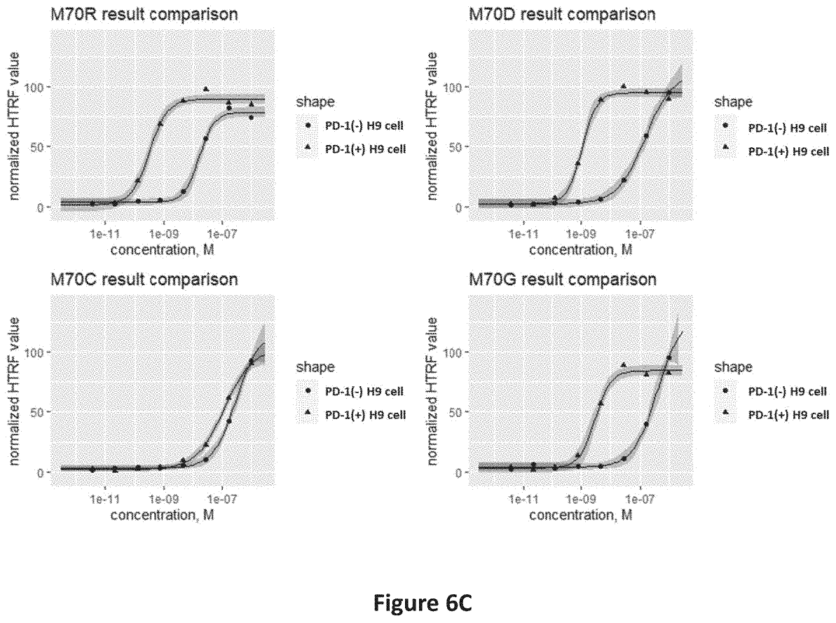

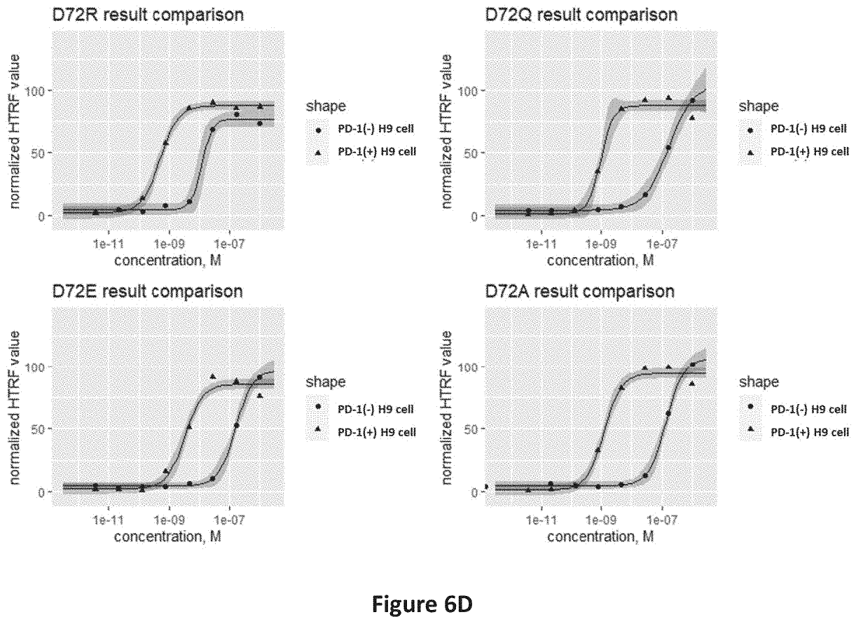

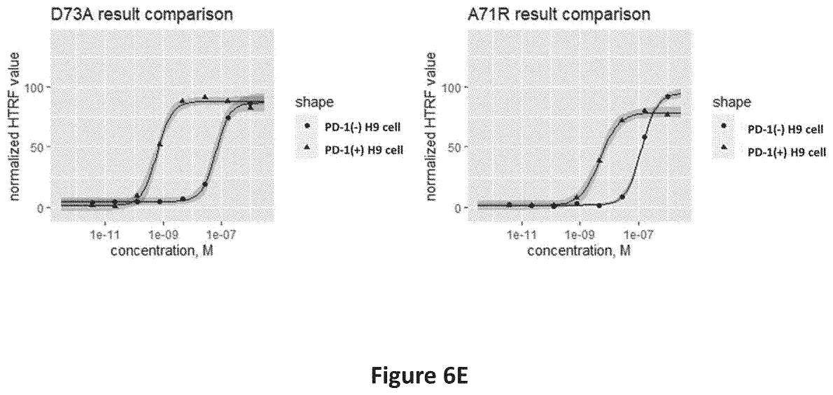

FIGS. 4, 5, and 6A-6E show that the moiety of an anti-PD-1 antibody of αPD-1IL21RαMutein/IL21 contributed to differences in the pharmacodynamics of αPD-1IL21RαMutein/IL21 on PD-1(+) or PD-1(−) H9 cells.

To be specific, the value of EC₅₀ of phosphorylation of STAT3 observed in αPD-1IL21RαMutein/IL21 treated PD-1(−) cells were higher than PD-1(+) cells, and the max efficacy was similar in both cell lines (Table 5).

TABLE 5
Summary of EC₅₀

| | PD-1(−) H9 cells | | PD-1(+) H9 cells | |
|---|---|---|---|---|
| Mutation | EC₅₀ (M) | sem | EC₅₀ (M) | sem |
| Wildtype | 2.58E−07 | 4.53E−08 | 1.77E−07 | 3.78E−08 |
| D72E | 1.57E−07 | 2.03E−08 | 3.29E−09 | 5.80E−10 |
| A71R | 1.29E−07 | 2.57E−08 | 4.87E−09 | 6.83E−10 |
| Y36G | 1.44E−07 | 3.80E−08 | 1.28E−08 | 3.68E−09 |
| M70Q | 1.75E−07 | 2.55E−08 | 6.14E−09 | 6.72E−10 |
| M70H | 1.98E−07 | 3.57E−08 | 6.93E−09 | 8.64E−10 |
| M70W | 1.90E−07 | 2.99E−08 | 6.66E−09 | 9.50E−10 |
| L94K | 2.53E−07 | 5.90E−08 | 1.03E−08 | 1.88E−09 |
| E38R | 2.13E−07 | 4.37E−08 | 1.30E−09 | 1.49E−10 |
| M70R | 1.63E−08 | 3.00E−09 | 3.21E−10 | 4.36E−11 |
| M70D | 1.51E−07 | 3.17E−08 | 1.06E−09 | 1.08E−10 |
| M70C | 2.96E−07 | 6.20E−08 | 1.15E−07 | 2.63E−08 |
| M70G | 4.19E−07 | 8.23E−08 | 2.91E−09 | 4.02E−10 |
| D72R | 1.20E−08 | 2.60E−09 | 5.04E−10 | 6.72E−11 |
| D72Q | 1.65E−07 | 3.35E−08 | 9.50E−10 | 1.17E−10 |
| D72A | 1.39E−07 | 1.59E−08 | 1.22E−09 | 1.71E−10 |
| D73A | 6.45E−08 | 9.86E−09 | 6.13E−10 | 8.81E−11 |
| D72E + E38A | 2.69E−08 | 6.03E−09 | 4.38E−10 | 7.87E−11 |

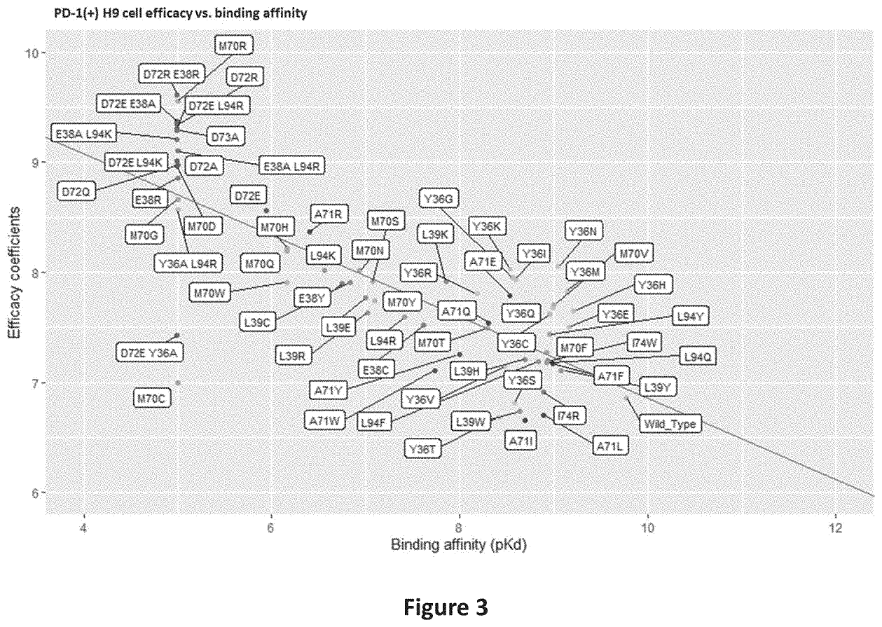

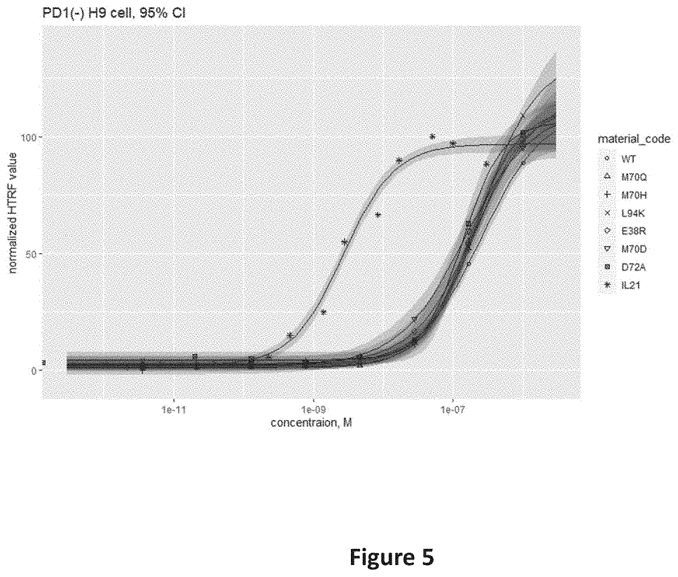

From the results of HTRF-based high-throughput screening, six variants of αPD-1IL21RαMutein/IL21, each containing a different mutein selected from E38R, M70D, M70H, M70Q, D72A, and L94K, were selected for further study (FIG. 3). Among these six variants, M70Q and M70H muteins showed efficacy comparable to wild type IL-21, distinguishing from a fusion protein containing an attenuated IL-21 mentioned above. The attenuated IL-21 showed less than 80% efficacy compared to wild type IL-21 (FIGS. 4 and 5).

These results demonstrate that IL-21Rα muteins of αPD-1IL21RαMutein/IL21 act as a capping molecule inhibiting IL-21 from binding to non-target cells, which is the reason for the low signal intensity in PD-1(−) cells. This shows that αPD-1IL21RαMutein/IL21 is an immunocytokine having high tissue specificity.

Besides, increasing specificity while maintaining efficacy of the drug substance by introducing a proper modification to a capping moiety to adjust specificity to its receptor is a unique advantage of our invention distinguishing from other drugs. αPD-1IL21RαMutein/IL21 shows characteristics of both full agonist and competitive antagonist.

7.5. SPR Full Kinetics Assay of Immunocytokines (αPD-1IL21Rα Mutein/IL21) against PD-1

Interaction between immunocytokine and human PD-1 (hPD-1) was determined by Surface Plasmon Resonance (SPR, Biacore 8K) analysis. Immunocytokines and hPD-1's affinity was tested by CMS sensor chip. 400 mM EDC and 100 mM NHS (Cytiva) were injected to CM5 sensor chip for 420 s with a flow rate of 10 μL/min as activator prior to injecting 25 μg/mL of anti-human Fc IgG in 10 mM NaAc (pH 4.5) to the channel 1-8 for 420 s at a flow rate of 10 μL/min. The chip was deactivated by 1M ethanolamine-HCl (Cytiva) at flow rate of 10 μL/min for 420 s.

Immunocytokines diluted in running buffer (1× HBS-EP+) were captured on to Fc2 via anti-human Fc IgG at flow rate of 10 μL/min for 40 s. Multiple cycle kinetics was used to perform the assay. The analyte hPD-1 at 7 different concentrations (0, 2.5, 5, 10, 20, 40, and 80 nM) and running buffer were injected orderly to Fc1-Fc2 at a flow rate of 30 μL/min for an association phase of 180 s, followed by 900 s dissociation. 10 mM glycine pH 1.5 was injected as regeneration buffer following every dissociation phase.

The sensorgrams from the reference channel Fc1 and the buffer channel were subtracted from the test sensorgrams. The experimental data was fitted by 1:1 binding model. Molecular weight of 17 kDa were used to calculate the molar concentration of hPD-1.

The SPR analysis demonstrated that the fusion of IL21RαWT or IL21RαMutein and IL-21 to the anti-PD-1 antibody did not affect the affinity of the anti-PD-1 antibody to PD-1 (FIG. 7A-7Q, Table 6).

TABLE 6

| No. | IL-21R Mutation Site | K_{D} (M) |
|---|---|---|
| 1 | WT | 8.41E−09 |
| 2 | Y36C | 8.36E−09 |
| 3 | Y36E | 8.32E−09 |
| 4 | Y36G | 8.51E−09 |
| 5 | Y36H | 9.02E−09 |
| 6 | Y36I | 8.74E−09 |
| 7 | Y36K | 8.23E−09 |
| 8 | Y36M | 8.32E−09 |
| 9 | Y36N | 8.20E−09 |
| 10 | Y36P | 8.44E−09 |
| 11 | Y36Q | 8.49E−09 |
| 12 | Y36R | 8.45E−09 |
| 13 | Y36S | 8.39E−09 |
| 14 | Y36T | 8.52E−09 |
| 15 | Y36V | 8.63E−09 |
| 16 | E38C | 9.06E−09 |
| 17 | E38R | 6.61E−09 |
| 18 | E38Y | 8.73E−09 |
| 19 | L39C | 8.61E−09 |
| 20 | L39E | 8.45E−09 |
| 21 | L39H | 8.24E−09 |
| 22 | L39K | 8.43E−09 |
| 23 | L39R | 8.62E−09 |
| 24 | L39W | 8.78E−09 |
| 25 | L39Y | 9.74E−09 |
| 26 | M70F | 9.01E−09 |
| 27 | M70H | 8.85E−09 |
| 28 | M70N | 8.78E−09 |
| 29 | M70Q | 8.85E−09 |
| 30 | M70S | 8.84E−09 |
| 31 | M70T | 8.49E−09 |
| 32 | M70V | 8.53E−09 |
| 33 | M70W | 8.86E−09 |
| 34 | M70Y | 8.81E−09 |
| 35 | A71E | 8.14E−09 |
| 36 | A71F | 8.70E−09 |
| 37 | A71I | 8.59E−09 |
| 38 | A71L | 8.87E−09 |
| 39 | A71Q | 8.51E−09 |
| 40 | A71R | 8.94E−09 |
| 41 | A71W | 9.12E−09 |
| 42 | A71Y | 8.94E−09 |
| 43 | I74R | 8.90E−09 |
| 44 | I74W | 8.94E−09 |
| 45 | L94F | 8.39E−09 |
| 46 | L94K | 8.90E−09 |
| 47 | L94Q | 8.90E−09 |
| 48 | L94R | 7.82E−09 |
| 49 | L94Y | 9.19E−09 |
| 50 | M70C | 7.91E−09 |
| 51 | M70D | 7.25E−09 |
| 52 | M70G | 8.42E−09 |
| 53 | M70R | 6.83E−09 |
| 54 | D72A | 6.88E−09 |
| 55 | D72E | 6.53E−09 |
| 56 | D72Q | 8.64E−09 |
| 57 | D72R | 8.03E−09 |
| 58 | D73A | 7.47E−09 |
| 59 | Y36A + L94R | 7.26E−09 |
| 60 | E38A + L94K | 7.70E−09 |
| 61 | E38A + L94R | 8.16E−09 |
| 62 | D72E + Y36A | 7.39E−09 |
| 63 | D72E + E38A | 7.19E−09 |
| 64 | D72E + L94K | 7.58E−09 |
| 65 | D72E + L94R | 8.01E−09 |
| 66 | D72R + E38R | 7.02E−09 |

7.6. Binding Affinity of Immunocytokines (αPD-1IL21RαMutein/IL21) to FcRn

The binding affinity of antibody-based protein drugs to FcRn is known to be highly associated with its half-life in vivo. The binding affinity of immunocytokines (αPD-1IL21RαMutein/IL21) to FcRn was measured using Bio-Layer Interferometry (BLI) system. As a control, the binding affinity of anti-PD-1 antibody which is not conjugated to IL-21 or IL-21 RαMutein was also measured.

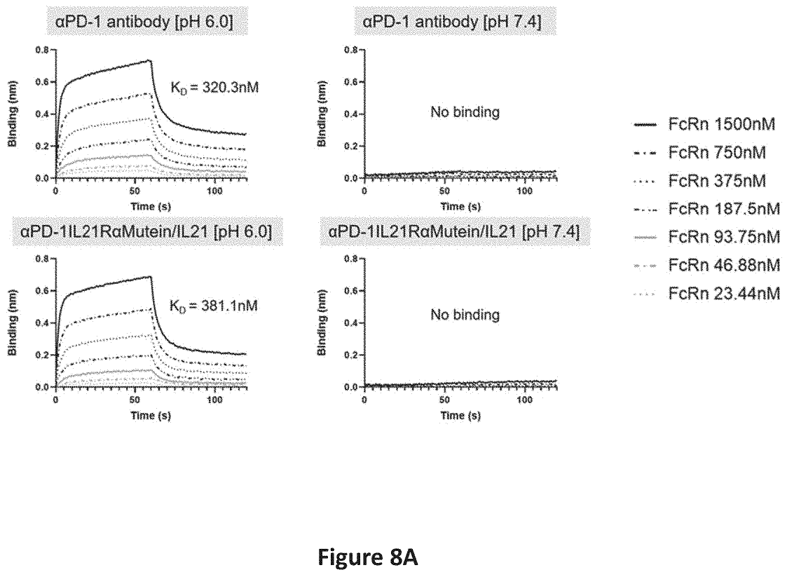

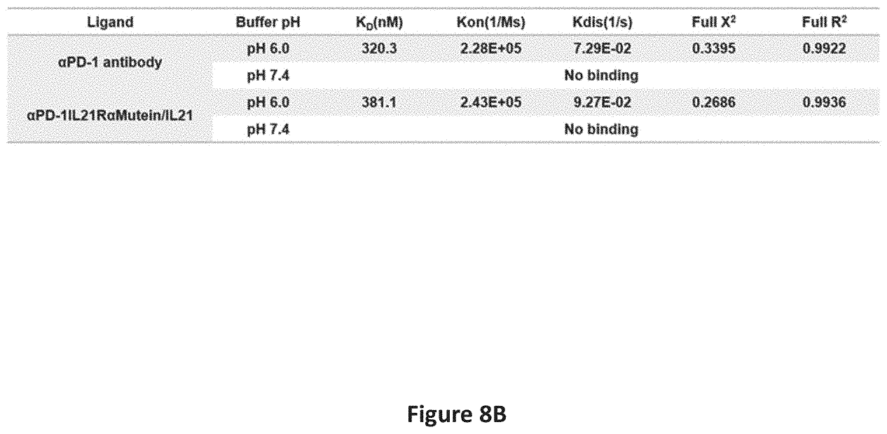

For the assay, FAB2G biosensor (Sartorius) was hydrated with a running buffer for 10 minutes in the 96 well plate (Corning). The ligands (anti-PD-1 antibody or Immunocytokine) were diluted with the running buffer to make a final concentration of 0.5 μg/ml for anti-PD-1 antibody and 2 μg/ml for immunocytokine. FAB2G biosensor was loaded with either anti-PD-1 antibody or Immunocytokine at 1.5nm level. After loading either anti-PD-1 antibody or Immunocytokine, the baseline was set by incubating the loaded sensor tip in the running buffer for 300 sec. Ligand loaded sensor tips were incubated in wells containing a 2-fold serial dilution of soluble, FcRn/B2M complex receptors. Association and dissociation were measured for 60 seconds or until a steady state was reached. The measurement data are provided in FIG. 8A.

The binding affinities of the anti-PD-1 antibody or Immunocytokine to FcRn were measured using Octet RED96e (ForteBio) instruments. Optimized Octet sample buffer (100 mM Sodium Phosphate, 300 mM NaCl, 0.05% Tween20) was used for sample dilution and all binding baseline, association, and dissociation steps at either pH of 6.0 or pH of 7.4. A buffer only blank curve was subtracted to correct any drift. The data were fit to a 1:1 binding model using ForteBio data analysis software 11.1 to determine the Kₒₙ, K_{off}, and K_{D}, which are provided in FIG. 8B.

The data show that the binding affinity of the immunocytokine to FcRn is not significantly different from the binding affinity of the anti-PD-1 antibody. This result suggests that the pharmacokinetic profile of the instant immunocytokine will benefit from FcRn binding ability, thus having a half-life sufficient to provide therapeutic effects.

7.7. In Vitro Tumor Killing Assay

To confirm the anti-tumor effects of the present immunocytokine (αPD-1IL21RαMutein/IL21), an increase in IFNγ expression level and a change in cytotoxicity of the CD8+ T cells that are treated with the present immunocytokine were tested. When the CD8+ T cells are co-cultured with autologous monocyte-derived DCs (moDCs) presenting specific antigens on their surfaces through MHC-peptide complexes, the tumor antigen educated CD8+ T cells (e.g., CTLs) can recognize and attack tumor cells expressing those antigens. The efficacy of the immunocytokines was confirmed by measuring fluorescent materials leaked from the tumor cells due to the death of tumor cells.

Specifically, human PBMCs were purchased from StemExpress (USA). Monocytes were isolated using Pan Monocyte Isolation Kit (Miltenyi Biotec) and were cultured for 7 days with 35 ng/mL recombinant human IL-4 (R&D Systems) and 50 ng/mL GM-CSF (R&D Systems) in RPMI1640 medium(Gibco) to differentiate the monocyte to dendritic cells (DCs). The premature monocyte-derived DCs were further matured for 3 days using 10 ng/mL recombinant human IL-6 (R&D Systems), 15 ng/mL IL-1β (R&D Systems), 40 ng/mL TGFα (R&D Systems), and 1 μg/mL PGE2 (PeproTech). During maturation, antigen peptides were loaded on the monocyte-derived DCs (moDCs). Autologous donor's CD8+ T cells were isolated using CD8⁺ T Cell Isolation Kit (Miltenyi Biotec) and were co-cultured with the matured moDCs for 10 days at a 10:1 cell number ratio. Culture medium supplemented with recombinant human IL-15 (R&D Systems) and recombinant human IL-7 (R&D Systems) were added every 2 or 3 days to sustain CTLs.

CTLs were then expanded using an anti-CD3ε antibody (R&D Systems), anti-CD28 antibody (R&D Systems), and recombinant human IL-2 (R&D Systems) for 5 days. During the expansion of CTLs (effector cell), the present immunocytokines (αPD-1IL21RαMutein/IL21 or αPD-1IL21RαWT/IL21) or controls (e.g., anti-PD-1 antibody) were treated at 500nM concentration.

7.7.1. Release of IFN-γ

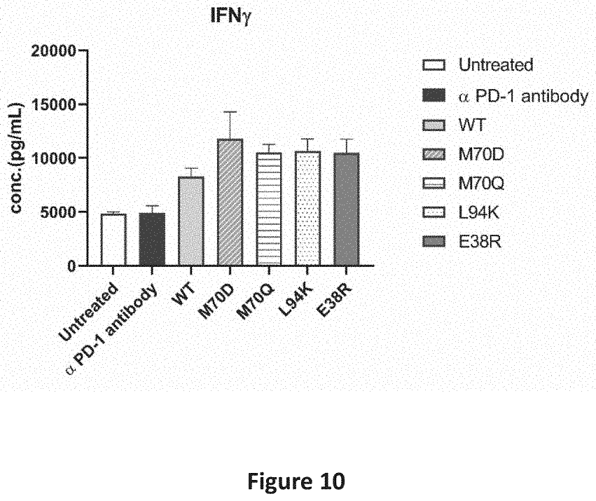

IFNγ levels in the culture supernatants were measured by ELISA using Human IFN-gamma DuoSet ELISA kit (R&D Systems). The results are provided in FIG. 10, confirming increased IFNγ release from CTL in response to immunocytokines (four variants of αPD-1IL21RαMutein/IL21, each containing a different mutein selected from M70D, M70Q, L94K, and E39R).

7.7.2. Cytotoxicity

To confirm tumor killing efficacy, Calcein AM(Invitrogen)-stained target cells (MeWo cell line or CMV pp65 gene transduced A375 cell line (A375_CMV)) were plated the day before co-culture with the expanded CTLs (effector cells). The effector cells were collected and loaded to the medium with target cells and cultured for 36 hours. The release of Calcein AM from the dead tumor cells were measured by detecting fluorescent signals at Ex 485 nm and Em 530 nm using FlexStation3 equipment.

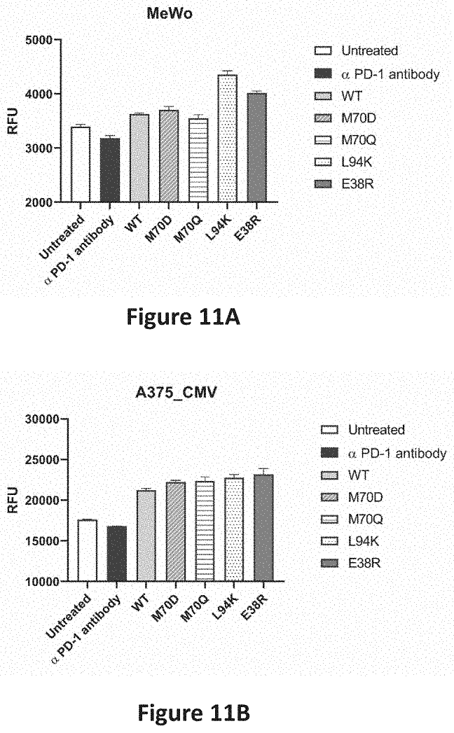

FIGS. 11A and 11B provide data from MeWo cell line and A375_CMV cell line, respectively. The data show that CTLs treated with αPD-1IL21RαMutein/IL21 showed better tumor-killing activity than the controls. This can be due to enhancement of effector function of CTLs by αPD-1IL21RαMutein/IL21. These suggest that immunocytokines provided here, αPD-1IL21RαMutein/IL21, can enhance anti-tumor activity when applied to cancer patients.

7.8. Immunocytokines against CTLA-4, TIGIT, LAG-3 (αCTLA-4L21RαMutein/IL21; αTIGITIL21RαMutein/IL21; or αLAG-3IL21RαMutein/IL21)

Two immunocytokines against each of three different targets, CTLA-4, TIGIT, and LAG-3 (αCTLA-4L21RαMutein/IL21; αTIGITIL21RαMutein/IL21; or αLAG-3IL21RαMutein/IL21) were generated by methods described above related to αPD-1L21RαMutein/IL21. The immunocytokines includes (i) a first chain comprising a heavy chain, G4S linker and IL-21RαMutein; (ii) a second chain comprising a heavy chain, G4S linker and a human IL-21; and (iii) two light chains, as specified in Table 7. The immunocytokines were successfully generated from the CHO cell lines, and the HTRF assay confirmed their functional activity of phosphorylation of STAT3 as described in 5.9.

TABLE 7

| | | Sequence of first | Sequence of second | |
|---|---|---|---|---|
| | IL-21Rα | chain (Heavy | chain (Heavy | |
| | Mutation | Chain-G4S Linker- | Chain-G4S Linker- | Sequence of two |
| | Site | IL-21RαMutein) | human IL-21) | light chains |
| Ipilimumab | WT | SEQ ID NO: 230 | SEQ ID NO: 229 | SEQ ID NO: 152 |
| | M70D | SEQ ID NO: 231 | SEQ ID NO: 229 | SEQ ID NO: 152 |
| | D72A | SEQ ID NO: 232 | SEQ ID NO: 229 | SEQ ID NO: 152 |
| Tiragolumab | WT | SEQ ID NO: 234 | SEQ ID NO: 233 | SEQ ID NO: 226 |
| | M70D | SEQ ID NO: 235 | SEQ ID NO: 233 | SEQ ID NO: 226 |
| | D72A | SEQ ID NO: 236 | SEQ ID NO: 233 | SEQ ID NO: 226 |
| Relatlimab | WT | SEQ ID NO: 238 | SEQ ID NO: 237 | SEQ ID NO: 228 |
| | M70D | SEQ ID NO: 239 | SEQ ID NO: 237 | SEQ ID NO: 228 |
| | D72A | SEQ ID NO: 240 | SEQ ID NO: 237 | SEQ ID NO: 228 |

7.9. Homogeneous Time-Resolved Fluorescence (HTRF) Phosphor-STAT3 Assay of Immunocytokines (αCTLA-4IL21RαMutein/IL21; αTIGITIL21RαMutein/IL21; and αLAG-3IL21RαMutein/IL21)

The immunocytokines against CTLA-4, TIGIT or LAG-3 were evaluated by measuring phosphorylation of STAT3 in HTRF-based high-throughput assay. Human cutaneous T lymphocyte cell lines (H9 (Cobioer), derivative of Hut78 cells) were grown in IMDM medium (Gibco) containing 20% fetal bovine serum (FBS, Gibco) and 1% penicillin/streptomycin (Sigma Aldrich) for H9. 3 μg/mL puromycin (Invivogen) was additionally added to the H9 cells. Subculture of cells was conducted every 48 hours to avoid high density which could arrest the cell cycle.

H9 cells were incubated with serum free media on overnight. After incubation, spin-down cells (with 125 g) were harvested with HBSS (Gibco) solution and seeded on white 96 well low volume plate (Cisbio) by 2.5×10⁴ cells/well/8 μL. Compounds for evaluation were prepared at 3× of the final concentration and applied to cells for 30 minutes at 37° C. The lysis buffer was added to the wells for 30 minutes and then reagents for HTRF reaction were treated following the manufacturer's protocol. After 24 hours, the HTRF reaction was measured by Flex Station 3 equipment.

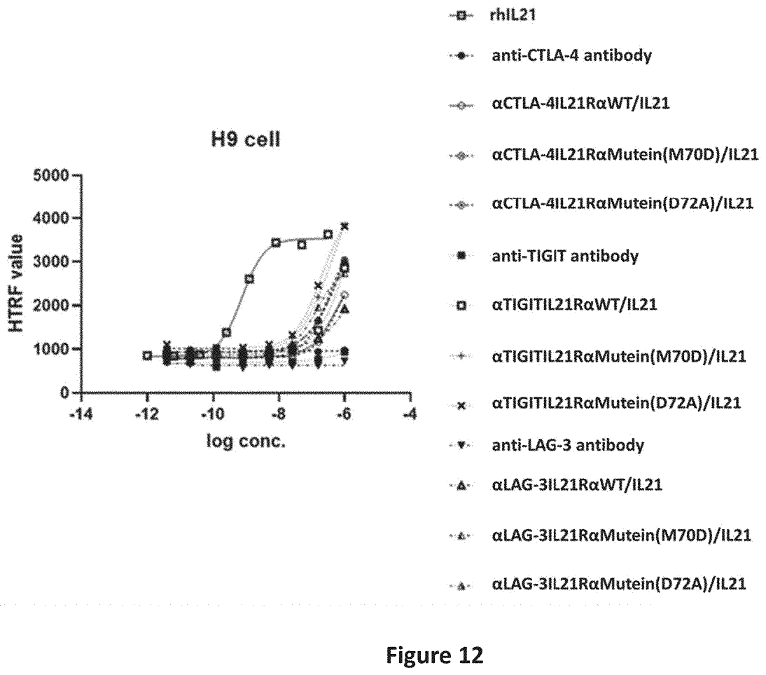

FIG. 12 and Table 8 provide data demonstrating that rhIL21, ABP-IL21RαWT/IL21, and ABP-IL21RαMutein/IL21 activated HTRF reaction. Among them, ABP-IL21RαWT/IL21 and ABP-IL21RαMutein/IL21 had significant lower activity than rhIL21, because of the masking effects of IL21RαWT or IL21RαMutein against IL21. As expected given that IL21RαWT has a higher affinity to IL21 compared to IL21RαMutein, the masking effects of IL21RαWT were greater than IL21RαMutein.

TABLE 8
Summary of EC₅₀

| | | | αCTLA-4 | αCTLA-4 | αCTLA-4 |
|---|---|---|---|---|---|
| | | anti-CTLA- | IL21Rα | IL21RαMutein | IL21RαMutein |
| | rhIL21 | 4 antibody | WT/IL21 | (M70D)/IL21 | (D72A)/IL21 |
| EC50(M) | 7.47E−10 | N/A | 6.524E−07 | 3.399E−07 | 5.143E−07 |
| EC50 ratio | 1.0 | N/A | 873.4 | 455.0 | 688.5 |
| | | anti- | αTIGIT | αTIGIT | αTIGIT |
| | | TIGIT | IL21Rα | IL21RαMutein | IL21RαMutein |
| | | antibody | WT/IL21 | (M70D)/IL21 | (D72A)/IL21 |
| EC50(M) | | N/A | 6.096E−07 | 2.017E−07 | 1.67E−07 |
| EC50 ratio | | N/A | 816.1 | 270.0 | 223.6 |
| | | | αLAG-3 | αLAG-3 | αLAG-3 |
| | | anti-LAG- | IL21Rα | IL21RαMutein | IL21RαMutein |
| | | 3 antibody | WT/IL21 | (M70D)/IL21 | (D72A)/IL21 |
| EC50(M) | | N/A | 2.73E−06 | 1.42E−07 | 4.538E−07 |
| EC50 ratio | | N/A | 3654.6 | 190.1 | 607.5 |

7.10. SPR Full Kinetics Assay of Immunocytokines (αCTLA-4IL21RαMutein/IL21; αTIGITIL21RαMutein/IL21; or αLAG-3IL21RαMutein/IL21)

Binding between the immunocytokines and their respective human target proteins (hCTLA-4, hTIGIT, or hLAG-3) was tested by Surface Plasmon Resonance (SPR) analysis. Affinities of the immunocytokines to their human ligands were tested by CM5 sensor chip. 400 mM EDC and 100 mM NHS (Cytiva) were injected to CM5 sensor chip for 420 s with a flow rate of 10 μL/min as activator prior to injecting 25 μg/mL of anti-human Fc IgG in 10 mM NaAc (pH 4.5) to the channel 1-8 for 420 s at a flow rate of 10 μL/min. The chip was deactivated by 1M ethanolamine-HCl (Cytiva) at flow rate of 10 μL/min for 420 s.

Immunocytokines diluted in running buffer (1× HBS-EP+) were captured on to Fc2 via anti-human Fc IgG at flow rate of 10 μL/min for 40 s. Multiple cycle kinetics was used to perform the assay. 6 concentrations (1.56, 3.13, 6.25, 12.5, 25, and 50 nM) of analyte hCTLA-4 (Acro Biosystems) or 6 concentrations (0.78, 1.56, 3.13, 6.25, 12.5, and 25 nM) of analyte hTIGIT (R&D systems) or 6 concentrations (0.31, 0.63, 1.25, 2.5, 5, and 10nM) of analyte hLAG-3 (Acro Biosystems) and running buffer were injected orderly to Fc1-Fc2 at a flow rate of 30 μL/min for an association phase of 180 s, followed by 900 s dissociation. 10 mM glycine pH 1.5 was injected as a regeneration buffer following every dissociation phase.

The sensorgrams for reference channel Fc1 and buffer channel were subtracted from the test sensorgrams. The experimental data was fitted by 1:1 binding model or heterogeneous ligand model.

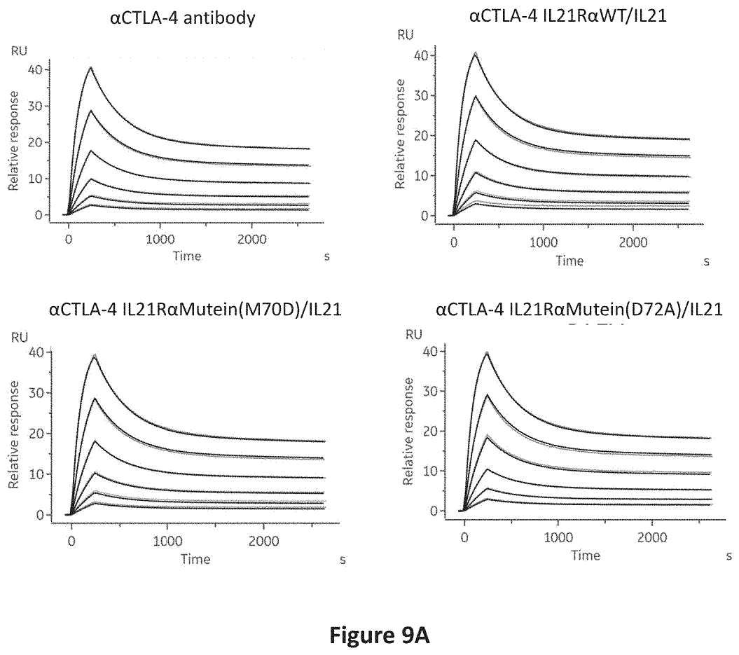

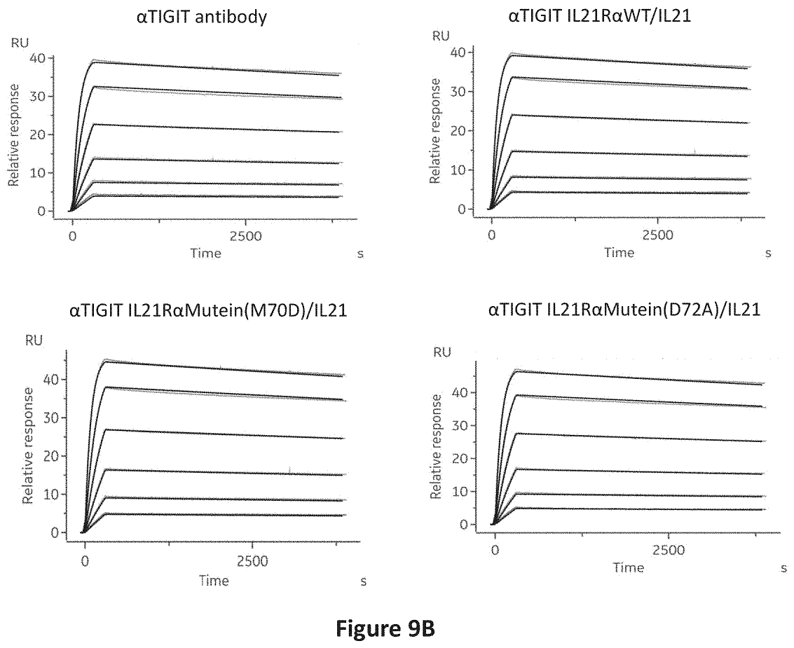

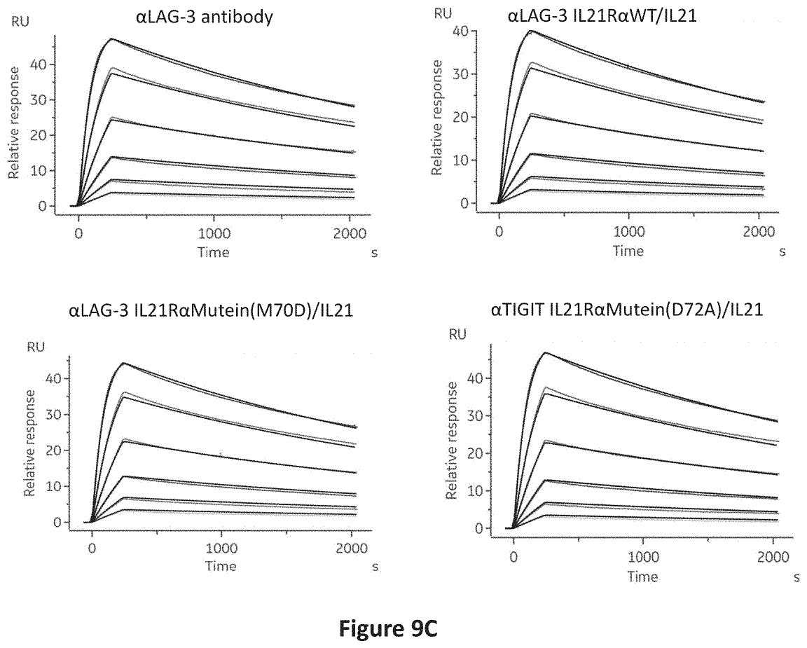

The SPR analysis demonstrated that the fusion of IL21RαWT or IL21RαMutein and IL-21 to the anti-CTLA-4, anti-TIGIT or anti-LAG-3 antibody did not affect the affinity of the anti-CTLA-4, anti-TIGIT or anti-LAG-3 antibody to its respective target (Table 9; FIGS. 9A, 9B and 9C).

TABLE 9
Affinity of immunocytokines (αCTLA-4IL21RαMutein/IL21;
αTIGITIL21RαMutein/IL21; or αLAG-3IL21RαMutein/IL21)
against targets (CTLA-4; TIGIT; or LAG-3)

| | Control | | | IL-21R | |
|---|---|---|---|---|---|
| No. | Antibody | K_{D} (M) | No. | Mutation Site | K_{D} (M) |
| 1 | Ipilimumab | 2.09E−08 | Against | | |
| | | | hCTLA-4 | | |
| 2 | Tiragolumab | 5.95E−11 | 1 | WT | 1.80E−08 |
| 3 | Relatlimab | 2.39E−10 | 2 | M70D | 1.81E−08 |

| N/A | 3 | D72A | 1.71E−08 |
|---|---|---|---|
| | Against | | |
| | hTIGIT | | |
| | 4 | WT | 5.17E−11 |
| | 5 | M70D | 5.37E−11 |
| | 6 | D72A | 5.60E−11 |
| | Against | | |
| | hLAG-3 | | |
| | 7 | WT | 2.64E−10 |
| | 8 | M70D | 2.49E−10 |
| | 9 | D72A | 2.55E−10 |

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification, are hereby incorporated by reference in their entirety, for all purposes.

### 9. SEQUENCE LISTING

Summary of Sequence Listing

| SEQ | |
|---|---|
| ID NO | Sequences |
| 1 | Nivolumab heavy chain |
| 2 | Nivolumab light chain |
| 3 | pembrolizumab heavy chain |
| 4 | pembrolizumab light chain |
| 5 | cemiplimab heavy chain |
| 6 | cemiplimab light chain |
| 7 | atezolizumab heavy chain |
| 8 | atezolizumab light chain |
| 9 | dostarlimab heavy chain |
| 10 | dostarlimab light chain |
| 11 | durvalumab heavy chain |
| 12 | durvalumab light chain |
| 13 | avelumab heavy chain |
| 14 | avelumab light chain |
| 15 | Wild type IL-21Rα (ectodomain; extracellular domain) |
| 16 | Human IgG1 Fc (100 Pro-330 Lys) |
| 17 | G4S linker |
| 18-99 | IL-21RαMutein |
| 100 | Human IL-21 |
| 101 | Second Chain (Heavy chain of anti-PD-1 antibody + linker + |
| | human IL-21) with knob mutation |
| 102 | Anti-PD-1 antibody, Light chain |
| 103 | Anti-PD-1 antibody, Heavy chain with hole mutation |
| 104-150 | αPD-1 + linker + IL21RαMutein |
| 151 | (Ipilimumab heavy chain) |
| 152 | (Ipilimumab light chain) |
| 153 | (tremelimumab heavy chain) |
| 154 | (tremelimumab light chain) |
| 155-169 | IL21RαMutein |
| 170-184 | Fc-Linker-IL21RαMutein |
| 185 | IgG1 Fc moiety (WT) |
| 186 | IgG2 Fc moiety (WT) |
| 187 | IgG3 Fc moiety (WT) |
| 188 | IgG4 Fc moiety (WT) |
| 189 | IGHG1 (Immunoglobulin heavy constant gamma 1) with |
| | ‘LALA’(L234A/L235A) mutation |
| 190 | IGHG4 (Immunoglobulin heavy constant gamma 4) with |
| | ‘SPLE’(S228P/L235E) mutation |
| 191 | αPD-1-linker- IL21RαWT |
| 192-209 | αPD-1 + linker + IL21RαMutein |
| 210 | Polynucleotide encoding IgG1-1IL21Rα (wild type) |
| 211 | Polynucleotide encoding αPD-1IL21Rα (wild type) |
| 212-217 | Flexible linkers |
| 218-224 | Rigid linkers |
| 225 | Tiragolumab Heavy chain |
| 226 | Tiragolumab light chain |
| 227 | Relatlimab Heavy chain |
| 228 | Relatlimab Light chain |
| 229 | Second Chain (Heavy chain of anti-CTLA-4 antibody + |
| | linker + human IL-21) with knob mutation |
| 230 | αCTLA-4 + linker + IL21RαWT |
| 231-232 | αCTLA-4 + linker + IL21RαMutein |
| 233 | Second Chain (Heavy chain of anti-TIGIT antibody + |
| | linker + human IL-21) with knob mutation |
| 234 | αTIGIT + linker + IL21RαWT |
| 235-236 | αTIGIT + linker + IL21RαMutein |
| 237 | Second Chain (Heavy chain of anti-LAG-3 antibody + |
| | linker + human IL-21) with knob mutation |
| 238 | αLAG-3 + linker + IL21RαWT |
| 239-240 | αLAG-3 + linker + IL21RαMutein |

| SEQ ID NO | Sequence |
|---|---|
| 1 | QVQLVESGGGVVQPGRSLRLDCKASGITFSNSGMHWVRQAPGKG |
| (Nivolumab | LEWVAVIWYDGSKRYY |
| heavy chain) | ADSVKGRFTISRDNSKNTLFLQMNSLRAEDTAVYYCATNDDYWG |
| | QGTLVTVSSASTKGPS |
| | VFPLAPCSRSTSESTAALGCLVKDYFPEPVTVSWNSGALTSGVHTF |
| | PAVLQSSGLYSLSS |
| | VVTVPSSSLGTKTYTCNVDHKPSNTKVDKRVESKYGPPCPPCPAPE |
| | FLGGPSVFLFPPKP |
| | KDTLMISRTPEVTCVVVDVSQEDPEVQFNWYVDGVEVHNAKTKP |
| | REEQFNSTYRVVSVLT |
| | VLHQDWLNGKEYKCKVSNKGLPSSIEKTISKAKGQPREPQVYTLP |
| | PSQEEMTKNQVSLTC |
| | LVKGFYPSDIAVEWESNGQPENNYKTTPPVLDSDGSFFLYSRLTVD |
| | KSRWQEGNVFSCSV |
| | MHEALHNHYTQKSLSLSLGK |
| 2 | EIVLTQSPATLSLSPGERATLSCRASQSVSSYLAWYQQKPGQAPRLL |
| (Nivolumab light | IYDASNRATGIPA |
| chain) | RFSGSGSGTDFTLTISSLEPEDFAVYYCQQSSNWPRTFGQGTKVEIK |
| | RTVAAPSVFIFPP |
| | SDEQLKSGTASVVCLLNNFYPREAKVQWKVDNALQSGNSQESVT |
| | EQDSKDSTYSLSSTLT |
| | LSKADYEKHKVYACEVTHQGLSSPVTKSFNRGEC |
| 3 | QVQLVQSGVEVKKPGASVKVSCKASGYTFTNYYMYWVRQAPGQ |
| (pembrolizumab | GLEWMGGINPSNGGTNF |
| heavy chain) | NEKFKNRVTLTTDSSTTTAYMELKSLQFDDTAVYYCARRDYRFDM |
| | GFDYWGQGTTVTVSS |
| | ASTKGPSVFPLAPCSRSTSESTAALGCLVKDYFPEPVTVSWNSGAL |
| | TSGVHTFPAVLQSS |
| | GLYSLSSVVTVPSSSLGTKTYTCNVDHKPSNTKVDKRVESKYGPP |
| | CPPCPAPEFLGGPSV |
| | FLFPPKPKDTLMISRTPEVTCVVVDVSQEDPEVQFNWYVDGVEVH |
| | NAKTKPREEQFNSTY |
| | RVVSVLTVLHQDWLNGKEYKCKVSNKGLPSSIEKTISKAKGQPRE |
| | PQVYTLPPSQEEMTK |
| | NQVSLTCLVKGFYPSDIAVEWESNGQPENNYKTTPPVLDSDGSFFL |
| | YSRLTVDKSRWQEG |
| | NVFSCSVMHEALHNHYTQKSLSLSLGK |
| 4 | EIVLTQSPATLSLSPGERATLSCRASKGVSTSGYSYLHWYQQKPGQ |
| (pembrolizumab | APRLLIYLASYLES |
| light chain) | GVPARFSGSGSGTDFTLTISSLEPEDFAVYYCQHSRDLPLTFGGGTK |
| | VEIKRTVAAPSVF |
| | IFPPSDEQLKSGTASVVCLLNNFYPREAKVQWKVDNALQSGNSQE |
| | SVTEQDSKDSTYSLS |
| | STLTLSKADYEKHKVYACEVTHQGLSSPVTKSFNRGEC |
| 5 | EVQLLESGGV LVQPGGSLRL SCAASGFTFS NFGMTWVRQA |
| (cemiplimab | PGKGLEWVSG ISGGGRDTYF ADSVKGRFTI SRDNSKNTLY |
| heavy chain) | LQMNSLKGED TAVYYCVKWG NIYFDYWGQG TLVTVSSAST |
| | KGPSVFPLAP CSRSTSESTA ALGCLVKDYF PEPVTVSWNS |
| | GALTSGVHTF PAVLQSSGLY SLSSVVTVPS SSLGTKTYTC |
| | NVDHKPSNTK VDKRVESKYG PPCPPCPAPE FLGGPSVFLF |
| | PPKPKDTLMI SRTPEVTCVV VDVSQEDPEV QFNWYVDGVE |
| | VHNAKTKPRE EQFNSTYRVV SVLTVLHQDW LNGKEYKCKV |
| | SNKGLPSSIE KTISKAKGQP REPQVYTLPP SQEEMTKNQV |
| | SLTCLVKGFY PSDIAVEWES NGQPENNYKT TPPVLDSDGS |
| | FFLYSRLTVD KSRWQEGNVF SCSVMHEALH NHYTQKSLSL |
| | SLGK |
| 6 | DIQMTQSPSS LSASVGDSIT ITCRASLSIN TFLNWYQQKP |
| (cemiplimab light | GKAPNLLIYA ASSLHGGVPS |
| chain) | RFSGSGSGTD FTLTIRTLQP EDFATYYCQQ SSNTPFTFGP |
| | GTVVDFRRTV AAPSVFIFPP |
| | SDEQLKSGTA SVVCLLNNFY PREAKVQWKV DNALQSGNSQ |
| | ESVTEQDSKD STYSLSSTLT |
| | LSKADYEKHK VYACEVTHQG LSSPVTKSFN RGEC |
| 7 | EVQLVESGGGLVQPGGSLRLSCAASGFTFSDSWIHWVRQAPGKGL |
| (atezolizumab | EWVAWISPYGGSTYY |
| heavy chain) | ADSVKGRFTISADTSKNTAYLQMNSLRAEDTAVYYCARRHWPGG |
| | FDYWGQGTLVTVSSAS |
| | TKGPSVFPLAPSSKSTSGGTAALGCLVKDYFPEPVTVSWNSGALTS |
| | GVHTFPAVLQSSGL |
| | YSLSSVVTVPSSSLGTQTYICNVNHKPSNTKVDKKVEPKSCDKTH |
| | TCPPCPAPELLGGPS |
| | VFLFPPKPKDTLMISRTPEVTCVVVDVSHEDPEVKFNWYVDGVEV |
| | HNAKTKPREEQYAST |
| | YRVVSVLTVLHQDWLNGKEYKCKVSNKALPAPIEKTISKAKGQPR |
| | EPQVYTLPPSREEMT |
| | KNQVSLTCLVKGFYPSDIAVEWESNGQPENNYKTTPPVLDSDGSFF |
| | LYSKLTVDKSRWQQ |
| | GNVFSCSVMHEALHNHYTQKSLSLSPGK |
| 8 | DIQMTQSPSSLSASVGDRVTITCRASQDVSTAVAWYQQKPGKAPK |
| (atezolizumab | LLIYSASFLYSGVPS |
| light chain) | RFSGSGSGTDFTLTISSLQPEDFATYYCQQYLYHPATFGQGTKVEIK |
| | RTVAAPSVFIFPP |
| | SDEQLKSGTASVVCLLNNFYPREAKVQWKVDNALQSGNSQESVT |
| | EQDSKDSTYSLSSTLT |
| | LSKADYEKHKVYACEVTHQGLSSPVTKSFNRGEC |
| 9 | EVQLLESGGGLVQPGGSLRLSCAASGFTFSSYDMSWVRQAPGKGL |
| (dostarlimab | EWVSTISGGGSYTYY |
| heavy chain) | QDSVKGRFTISRDNSKNTLYLQMNSLRAEDTAVYYCASPYYAMD |
| | YWGQGTTVTVSSASTK |
| | GPSVFPLAPCSRSTSESTAALGCLVKDYFPEPVTVSWNSGALTSGV |
| | HTFPAVLQSSGLYS |
| | LSSVVTVPSSSLGTKTYTCNVDHKPSNTKVDKRVESKYGPPCPPCP |
| | APEFLGGPSVFLFP |
| | PKPKDTLMISRTPEVTCVVVDVSQEDPEVQFNWYVDGVEVHNAK |
| | TKPREEQFNSTYRVVS |
| | VLTVLHQDWLNGKEYKCKVSNKGLPSSIEKTISKAKGQPREPQVY |
| | TLPPSQEEMTKNQVS |
| | LTCLVKGFYPSDIAVEWESNGQPENNYKTTPPVLDSDGSFFLYSRL |
| | TVDKSRWQEGNVFS |
| | CSVMHEALHNHYTQKSLSLSLGK |
| 10 | DIQLTQSPSFLSAYVGDRVTITCKASQDVGTAVAWYQQKPGKAPKL |
| (dostarlimab light | LIYWASTLHTGVPS |
| chain) | RFSGSGSGTEFTLTISSLQPEDFATYYCQHYSSYPWTFGQGTKLEIK |
| | RTVAAPSVFIFPP |
| | SDEQLKSGTASVVCLLNNFYPREAKVQWKVDNALQSGNSQESVT |
| | EQDSKDSTYSLSSTLT |
| | LSKADYEKHKVYACEVTHQGLSSPVTKSFNRGEC |
| 11 | EVQLVESGGGLVQPGGSLRLSCAASGFTFSRYWMSWVRQAPGKG |
| (durvalumab | LEWVANIKQDGSEKYY |
| heavy chain) | VDSVKGRFTISRDNAKNSLYLQMNSLRAEDTAVYYCAREGGWFG |
| | ELAFDYWGQGTLVTVS |
| | SASTKGPSVFPLAPSSKSTSGGTAALGCLVKDYFPEPVTVSWNSGA |
| | LTSGVHTFPAVLQS |
| | SGLYSLSSVVTVPSSSLGTQTYICNVNHKPSNTKVDKRVEPKSCDK |
| | THTCPPCPAPEFEG |
| | GPSVFLFPPKPKDTLMISRTPEVTCVVVDVSHEDPEVKFNWYVDG |
| | VEVHNAKTKPREEQY |
| | NSTYRVVSVLTVLHQDWLNGKEYKCKVSNKALPASIEKTISKAKG |
| | QPREPQVYTLPP SRE |
| | EMTKNQVSLTCLVKGFYPSDIAVEWESNGQPENNYKTTPPVLDSD |
| | GSFFLYSKLTVDKSR |
| | WQQGNVFSCSVMHEALHNHYTQKSLSLSPGK |
| 12 | EIVLTQSPGTLSLSPGERATLSCRASQRVSSSYLAWYQQKPGQAPR |
| (durvalumab light | LLIYDASSRATGIP |
| chain) | DRFSGSGSGTDFTLTISRLEPEDFAVYYCQQYGSLPWTFGQGTKVE |
| | IKRTVAAPSVFIFP |
| | PSDEQLKSGTASVVCLLNNFYPREAKVQWKVDNALQSGNSQESV |
| | TEQDSKDSTYSLSSTL |
| | TLSKADYEKHKVYACEVTHQGLSSPVTKSFNRGEC |
| 13 | EVQLLESGGGLVQPGGSLRLSCAASGFTFSSYIMMWVRQAPGKGL |
| (avelumab heavy | EWVSSIYPSGGITFY |
| chain) | ADTVKGRFTISRDNSKNTLYLQMNSLRAEDTAVYYCARIKLGTVT |
| | TVDYWGQGTLVTVSS |
| | ASTKGPSVFPLAPSSKSTSGGTAALGCLVKDYFPEPVTVSWNSGAL |
| | TSGVHTFPAVLQSS |
| | GLYSLSSVVTVPSSSLGTQTYICNVNHKPSNTKVDKKVEPKSCDKT |
| | HTCPPCPAPELLGG |
| | PSVFLFPPKPKDTLMISRTPEVTCVVVDVSHEDPEVKFNWYVDGV |
| | EVHNAKTKPREEQYN |
| | STYRVVSVLTVLHQDWLNGKEYKCKVSNKALPAPIEKTISKAKGQ |
| | PREPQVYTLPPSRDE |
| | LTKNQVSLTCLVKGFYPSDIAVEWESNGQPENNYKTTPPVLDSDGS |
| | FFLYSKLTVDKSRW |
| | QQGNVFSCSVMHEALHNHYTQKSLSLSPGK |
| 14 | QSALTQPASVSGSPGQSITISCTGTSSDVGGYNYVSWYQQHPGKAP |
| (avelumab light | KLMIYDVSNRPSGV |
| chain) | SNRFSGSKSGNTASLTISGLQAEDEADYYCSSYTSSSTRVFGTGTK |
| | VTVLGQPKANPTVT |
| | LFPPSSEELQANKATLVCLISDFYPGAVTVAWKADGSPVKAGVETT |
| | KPSKQSNNKYAASS |
| | YLSLTPEQWKSHRSYSCQVTHEGSTVEKTVAPTECS |
| 15 | CPDLVCYTDYLQTVICILEMWNLHPSTLTLTWQDQYEELKDEATS |
| IL-21RαWT | CSLHRSAHNATHATY |
| | TCHMDVFHFMADDIFSVNITDQSGNYSQECGSFLLAESIKPAPPFN |
| | VTVTFSGQYNISWR |
| | SDYEDPAFYMLKGKLQYELQYRNRGDPWAVSPRRKLISVDSRSVS |
| | LLPLEFRKDSSYELQVRAGPMPGSSYQGTWSEWSDPVIFQTQSEE |
| | LKE |
| 16 | PKSCDKTHTCPPCPAPELLGGPSVFLFPPKPKDTLMISRTPEVTCVV |
| Human IgG1 Fc | VDVSHEDPEVKFNWYVDGVEVHNAKTKPREEQYNSTYRVVSVLT |
| (100 Pro-330 | VLHQDWLNGKEYKCKVSNKALPAPIEKTISKAKGQPREPQVYTLP |
| Lys) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGK |
| 17 | GGGGSGGGGSGGGGS |
| G4S linker | |
| 18 | CPDLVCYTDYLQTVICILEMWNLHPSTLTLTWQDKYEELKDEATS |
| Q35K | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 19 | CPDLVCYTDYLQTVICILEMWNLHPSTLTLTWQDRYEELKDEATS |
| Q35R | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 20 | CPDLVCYTDYLQTVICILEMWNLHPSTLTLTWQDYYEELKDEATS |
| Q35Y | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 21 | CPDLVCYTDYLQTVICILEMWNLHPSTLTLTWQDQCEELKDEATS |
| Y36C | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 22 | CPDLVCYTDYLQTVICILEMWNLHPSTLTLTWQDQEEELKDEATS |
| Y36E | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 23 | CPDLVCYTDYLQTVICILEMWNLHPSTLTLTWQDQGEELKDEATS |
| Y36G | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 24 | CPDLVCYTDYLQTVICILEMWNLHPSTLTLTWQDQHEELKDEATS |
| Y36H | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 25 | CPDLVCYTDYLQTVICILEMWNLHPSTLTLTWQDQIEELKDEATSC |
| Y36I | SLHRSAHNATHATYTCHMDVFHFMADDIFSVNITDQSGNYSQECG |
| (IL-21Rα mutein) | SFLLAESIKPAPPFNVTVTFSGQYNISWRSDYEDPAFYMLKGKLQY |
| | ELQYRNRGDPWAVSPRRKLISVDSRSVSLLPLEFRKDSSYELQVRA |
| | GPMPGSSYQGTWSEWSDPVIFQTQSEELKE |
| 26 | CPDLVCYTDYLQTVICILEMWNLHPSTLTLTWQDQKEELKDEATS |
| Y36K | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 27 | CPDLVCYTDYLQTVICILEMWNLHPSTLTLTWQDQMEELKDEATS |
| Y36M | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 28 | CPDLVCYTDYLQTVICILEMWNLHPSTLTLTWQDQNEELKDEATS |
| Y36N | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 29 | CPDLVCYTDYLQTVICILEMWNLHPSTLTLTWQDQPEELKDEATSC |
| Y36P | SLHRSAHNATHATYTCHMDVFHFMADDIFSVNITDQSGNYSQECG |
| (IL-21Rα mutein) | SFLLAESIKPAPPFNVTVTFSGQYNISWRSDYEDPAFYMLKGKLQY |
| | ELQYRNRGDPWAVSPRRKLISVDSRSVSLLPLEFRKDSSYELQVRA |
| | GPMPGSSYQGTWSEWSDPVIFQTQSEELKE |
| 30 | CPDLVCYTDYLQTVICILEMWNLHPSTLTLTWQDQQEELKDEATS |
| Y36Q | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 31 | CPDLVCYTDYLQTVICILEMWNLHPSTLTLTWQDQREELKDEATS |
| Y36R | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 32 | CPDLVCYTDYLQTVICILEMWNLHPSTLTLTWQDQSEELKDEATSC |
| Y36S | SLHRSAHNATHATYTCHMDVFHFMADDIFSVNITDQSGNYSQECG |
| (IL-21Rα mutein) | SFLLAESIKPAPPFNVTVTFSGQYNISWRSDYEDPAFYMLKGKLQY |
| | ELQYRNRGDPWAVSPRRKLISVDSRSVSLLPLEFRKDSSYELQVRA |
| | GPMPGSSYQGTWSEWSDPVIFQTQSEELKE |
| 33 | CPDLVCYTDYLQTVICILEMWNLHPSTLTLTWQDQTEELKDEATS |
| Y36T | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 34 | CPDLVCYTDYLQTVICILEMWNLHPSTLTLTWQDQVEELKDEATS |
| Y36V | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 35 | CPDLVCYTDYLQTVICILEMWNLHPSTLTLTWQDQYECLKDEATS |
| E38C | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 36 | CPDLVCYTDYLQTVICILEMWNLHPSTLTLTWQDQYEKLKDEATS |
| E38K | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 37 | CPDLVCYTDYLQTVICILEMWNLHPSTLTLTWQDQYERLKDEATS |
| E38R | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 38 | CPDLVCYTDYLQTVICILEMWNLHPSTLTLTWQDQYEYLKDEATS |
| E38Y | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 39 | CPDLVCYTDYLQTVICILEMWNLHPSTLTLTWQDQYEECKDEATS |
| L39C | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 40 | CPDLVCYTDYLQTVICILEMWNLHPSTLTLTWQDQYEEEKDEATS |
| L39E | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 41 | CPDLVCYTDYLQTVICILEMWNLHPSTLTLTWQDQYEEFKDEATS |
| L39F | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 42 | CPDLVCYTDYLQTVICILEMWNLHPSTLTLTWQDQYEEHKDEATS |
| L39H | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 43 | CPDLVCYTDYLQTVICILEMWNLHPSTLTLTWQDQYEEKKDEATS |
| L39K | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 44 | CPDLVCYTDYLQTVICILEMWNLHPSTLTLTWQDQYEERKDEATS |
| L39R | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 45 | CPDLVCYTDYLQTVICILEMWNLHPSTLTLTWQDQYEEWKDEATS |
| L39W | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 46 | CPDLVCYTDYLQTVICILEMWNLHPSTLTLTWQDQYEEYKDEATS |
| L39Y | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 47 | CPDLVCYTDYLQTVICILEMWNLHPSTLTLTWQDQYEELKDEATS |
| M70C | CSLHRSAHNATHATYTCHMDVFHFCADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 48 | CPDLVCYTDYLQTVICILEMWNLHPSTLTLTWQDQYEELKDEATS |
| M70D | CSLHRSAHNATHATYTCHMDVFHFDADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 49 | CPDLVCYTDYLQTVICILEMWNLHPSTLTLTWQDQYEELKDEATS |
| M70F | CSLHRSAHNATHATYTCHMDVFHFFADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 50 | CPDLVCYTDYLQTVICILEMWNLHPSTLTLTWQDQYEELKDEATS |
| M70G | CSLHRSAHNATHATYTCHMDVFHFGADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 51 | CPDLVCYTDYLQTVICILEMWNLHPSTLTLTWQDQYEELKDEATS |
| M70H | CSLHRSAHNATHATYTCHMDVFHFHADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 52 | CPDLVCYTDYLQTVICILEMWNLHPSTLTLTWQDQYEELKDEATS |
| M70K | CSLHRSAHNATHATYTCHMDVFHFKADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 53 | CPDLVCYTDYLQTVICILEMWNLHPSTLTLTWQDQYEELKDEATS |
| M70L | CSLHRSAHNATHATYTCHMDVFHFLADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 54 | CPDLVCYTDYLQTVICILEMWNLHPSTLTLTWQDQYEELKDEATS |
| M70N | CSLHRSAHNATHATYTCHMDVFHFNADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 55 | CPDLVCYTDYLQTVICILEMWNLHPSTLTLTWQDQYEELKDEATS |
| M70Q | CSLHRSAHNATHATYTCHMDVFHFQADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 56 | CPDLVCYTDYLQTVICILEMWNLHPSTLTLTWQDQYEELKDEATS |
| M70R | CSLHRSAHNATHATYTCHMDVFHFRADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 57 | CPDLVCYTDYLQTVICILEMWNLHPSTLTLTWQDQYEELKDEATS |
| M70S | CSLHRSAHNATHATYTCHMDVFHFSADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 58 | CPDLVCYTDYLQTVICILEMWNLHPSTLTLTWQDQYEELKDEATS |
| M70T | CSLHRSAHNATHATYTCHMDVFHFTADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 59 | CPDLVCYTDYLQTVICILEMWNLHPSTLTLTWQDQYEELKDEATS |
| M70V | CSLHRSAHNATHATYTCHMDVFHFVADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 60 | CPDLVCYTDYLQTVICILEMWNLHPSTLTLTWQDQYEELKDEATS |
| M70W | CSLHRSAHNATHATYTCHMDVFHFWADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 61 | CPDLVCYTDYLQTVICILEMWNLHPSTLTLTWQDQYEELKDEATS |
| M70Y | CSLHRSAHNATHATYTCHMDVFHFYADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 62 | CPDLVCYTDYLQTVICILEMWNLHPSTLTLTWQDQYEELKDEATS |
| A71E | CSLHRSAHNATHATYTCHMDVFHFMEDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 63 | CPDLVCYTDYLQTVICILEMWNLHPSTLTLTWQDQYEELKDEATS |
| A71F | CSLHRSAHNATHATYTCHMDVFHFMFDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 64 | CPDLVCYTDYLQTVICILEMWNLHPSTLTLTWQDQYEELKDEATS |
| A71I | CSLHRSAHNATHATYTCHMDVFHFMIDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 65 | CPDLVCYTDYLQTVICILEMWNLHPSTLTLTWQDQYEELKDEATS |
| A71L | CSLHRSAHNATHATYTCHMDVFHFMLDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 66 | CPDLVCYTDYLQTVICILEMWNLHPSTLTLTWQDQYEELKDEATS |
| A71Q | CSLHRSAHNATHATYTCHMDVFHFMQDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 67 | CPDLVCYTDYLQTVICILEMWNLHPSTLTLTWQDQYEELKDEATS |
| A71R | CSLHRSAHNATHATYTCHMDVFHFMRDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 68 | CPDLVCYTDYLQTVICILEMWNLHPSTLTLTWQDQYEELKDEATS |
| A71W | CSLHRSAHNATHATYTCHMDVFHFMWDDIFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 69 | CPDLVCYTDYLQTVICILEMWNLHPSTLTLTWQDQYEELKDEATS |
| A71Y | CSLHRSAHNATHATYTCHMDVFHFMYDDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 70 | CPDLVCYTDYLQTVICILEMWNLHPSTLTLTWQDQYEELKDEATS |
| D72A | CSLHRSAHNATHATYTCHMDVFHFMAADIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 71 | CPDLVCYTDYLQTVICILEMWNLHPSTLTLTWQDQYEELKDEATS |
| D72C | CSLHRSAHNATHATYTCHMDVFHFMACDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 72 | CPDLVCYTDYLQTVICILEMWNLHPSTLTLTWQDQYEELKDEATS |
| D72E | CSLHRSAHNATHATYTCHMDVFHFMAEDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 73 | CPDLVCYTDYLQTVICILEMWNLHPSTLTLTWQDQYEELKDEATS |
| D72F | CSLHRSAHNATHATYTCHMDVFHFMAFDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 74 | CPDLVCYTDYLQTVICILEMWNLHPSTLTLTWQDQYEELKDEATS |
| D72G | CSLHRSAHNATHATYTCHMDVFHFMAGDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 75 | CPDLVCYTDYLQTVICILEMWNLHPSTLTLTWQDQYEELKDEATS |
| D72H | CSLHRSAHNATHATYTCHMDVFHFMAHDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 76 | CPDLVCYTDYLQTVICILEMWNLHPSTLTLTWQDQYEELKDEATS |
| D72I | CSLHRSAHNATHATYTCHMDVFHFMAIDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 77 | CPDLVCYTDYLQTVICILEMWNLHPSTLTLTWQDQYEELKDEATS |
| D72K | CSLHRSAHNATHATYTCHMDVFHFMAKDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 78 | CPDLVCYTDYLQTVICILEMWNLHPSTLTLTWQDQYEELKDEATS |
| D72L | CSLHRSAHNATHATYTCHMDVFHFMALDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 79 | CPDLVCYTDYLQTVICILEMWNLHPSTLTLTWQDQYEELKDEATS |
| D72M | CSLHRSAHNATHATYTCHMDVFHFMAMDIFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 80 | CPDLVCYTDYLQTVICILEMWNLHPSTLTLTWQDQYEELKDEATS |
| D72Q | CSLHRSAHNATHATYTCHMDVFHFMAQDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 81 | CPDLVCYTDYLQTVICILEMWNLHPSTLTLTWQDQYEELKDEATS |
| D72R | CSLHRSAHNATHATYTCHMDVFHFMARDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 82 | CPDLVCYTDYLQTVICILEMWNLHPSTLTLTWQDQYEELKDEATS |
| D72W | CSLHRSAHNATHATYTCHMDVFHFMAWDIFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 83 | CPDLVCYTDYLQTVICILEMWNLHPSTLTLTWQDQYEELKDEATS |
| D72Y | CSLHRSAHNATHATYTCHMDVFHFMAYDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 84 | CPDLVCYTDYLQTVICILEMWNLHPSTLTLTWQDQYEELKDEATS |
| D73C | CSLHRSAHNATHATYTCHMDVFHFMADCIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 85 | CPDLVCYTDYLQTVICILEMWNLHPSTLTLTWQDQYEELKDEATS |
| D73A | CSLHRSAHNATHATYTCHMDVFHFMADAIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 86 | CPDLVCYTDYLQTVICILEMWNLHPSTLTLTWQDQYEELKDEATS |
| D73E | CSLHRSAHNATHATYTCHMDVFHFMADEIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 87 | CPDLVCYTDYLQTVICILEMWNLHPSTLTLTWQDQYEELKDEATS |
| D73H | CSLHRSAHNATHATYTCHMDVFHFMADHIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 88 | CPDLVCYTDYLQTVICILEMWNLHPSTLTLTWQDQYEELKDEATS |
| D73K | CSLHRSAHNATHATYTCHMDVFHFMADKIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 89 | CPDLVCYTDYLQTVICILEMWNLHPSTLTLTWQDQYEELKDEATS |
| D73R | CSLHRSAHNATHATYTCHMDVFHFMADRIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 90 | CPDLVCYTDYLQTVICILEMWNLHPSTLTLTWQDQYEELKDEATS |
| D73W | CSLHRSAHNATHATYTCHMDVFHFMADWIFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 91 | CPDLVCYTDYLQTVICILEMWNLHPSTLTLTWQDQYEELKDEATS |
| D73Y | CSLHRSAHNATHATYTCHMDVFHFMADYIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 92 | CPDLVCYTDYLQTVICILEMWNLHPSTLTLTWQDQYEELKDEATS |
| I74H | CSLHRSAHNATHATYTCHMDVFHFMADDHFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 93 | CPDLVCYTDYLQTVICILEMWNLHPSTLTLTWQDQYEELKDEATS |
| I74K | CSLHRSAHNATHATYTCHMDVFHFMADDKFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 94 | CPDLVCYTDYLQTVICILEMWNLHPSTLTLTWQDQYEELKDEATS |
| I74R | CSLHRSAHNATHATYTCHMDVFHFMADDRFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 95 | CPDLVCYTDYLQTVICILEMWNLHPSTLTLTWQDQYEELKDEATS |
| I74W | CSLHRSAHNATHATYTCHMDVFHFMADDWFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 96 | CPDLVCYTDYLQTVICILEMWNLHPSTLTLTWQDQYEELKDEATS |
| L94F | CSLHRSAHNATHATYTCHMDVFHFMADDIFSVNITDQSGNYSQEC |
| (IL-21Rα mutein) | GSFFLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 97 | CPDLVCYTDYLQTVICILEMWNLHPSTLTLTWQDQYEELKDEATS |
| L94K | CSLHRSAHNATHATYTCHMDVFHFMADDIFSVNITDQSGNYSQEC |
| (IL-21Rα mutein) | GSFKLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 98 | CPDLVCYTDYLQTVICILEMWNLHPSTLTLTWQDQYEELKDEATS |
| L94Q | CSLHRSAHNATHATYTCHMDVFHFMADDIFSVNITDQSGNYSQEC |
| (IL-21Rα mutein) | GSFQLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 99 | CPDLVCYTDYLQTVICILEMWNLHPSTLTLTWQDQYEELKDEATS |
| L94R | CSLHRSAHNATHATYTCHMDVFHFMADDIFSVNITDQSGNYSQEC |
| (IL-21Rα mutein) | GSFRLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 100 | QDRHMIRMRQLIDIVDQLKNYVNDLVPEFLPAPEDVETNCEWSAF |
| Human IL-21 | SCFQKAQLKSANTGNNERIINVSIKKLKRKPPSTNAGRRQKHRLTC |
| | PSCDSYEKKPPKEFLERFKSLLQKMIHQHLSSRTHGSEDS |
| 101 | QVQLVESGGGVVQPGRSLRLDCKASGITFSNSGMHWVRQAPGKG |
| second chain | LEWVAVIWYDGSKRYYADSVKGRFTISRDNSKNTLFLQMNSLRAE |
| (Heavy chain of | DTAVYYCATNDDYWGQGTLVTVSSASTKGPSVFPLAPCSRSTSEST |
| anti-PD-1 | AALGCLVKDYFPEPVTVSWNSGALTSGVHTFPAVLQSSGLYSLSSV |
| antibody + | VTVPSSSLGTKTYTCNVDHKPSNTKVDKRVESKYGPPCPPCPAPEF |
| linker + | LGGPSVFLFPPKPKDTLMISRTPEVTCVVVDVSQEDPEVQFNWYV |
| human IL-21 with | DGVEVHNAKTKPREEQFNSTYRVVSVLTVLHQDWLNGKEYKCK |
| a knob mutation | VSNKGLPSSIEKTISKAKGQPREPQVYTLPPSQEEMTKNQVSLWCL |
| | VKGFYPSDIAVEWESNGQPENNYKTTPPVLDSDGSFFLYSRLTVDK |
| | SRWQEGNVFSCSVMHEALHNHYTQKSLSLSLGGGGGSGGGGSGG |
| | GGSQDRHMIRMRQLIDIVDQLKNYVNDLVPEFLPAPEDVETNCEW |
| | SAFSCFQKAQLKSANTGNNERIINVSIKKLKRKPPSTNAGRRQKHR |
| | LTCPSCDSYEKKPPKEFLERFKSLLQKMIHQHLSSRTHGSEDS |
| 102 | EIVLTQSPATLSLSPGERATLSCRASQSVSSYLAWYQQKPGQAPRLL |
| Anti-PD-1 | IYDASNRATGIPARFSGSGSGTDFTLTISSLEPEDFAVYYCQQSSNW |
| antibody, Light | PRTFGQGTKVEIKRTVAAPSVFIFPPSDEQLKSGTASVVCLLNNFYP |
| chain | REAKVQWKVDNALQSGNSQESVTEQDSKDSTYSLSSTLTLSKAD |
| | YEKHKVYACEVTHQGLSSPVTKSFNRGEC |
| 103 | QVQLVESGGGVVQPGRSLRLDCKASGITFSNSGMHWVRQAPGKG |
| Anti-PD-1 | LEWVAVIWYDGSKRYYADSVKGRFTISRDNSKNTLFLQMNSLRAE |
| antibody, Heavy | DTAVYYCATNDDYWGQGTLVTVSSASTKGPSVFPLAPCSRSTSEST |
| chain with a hole | AALGCLVKDYFPEPVTVSWNSGALTSGVHTFPAVLQSSGLYSLSSV |
| mutation | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLG |
| 104 | QVQLVESGGGVVQPGRSLRLDCKASGITFSNSGMHWVRQAPGKG |
| Y36C | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQCEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 105 | QVQLVESGGGVVQPGRSLRLDCKASGITFSNSGMHWVRQAPGKG |
| Y36E | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQEEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 106 | QVQLVESGGGVVQPGRSLRLDCKASGITFSNSGMHWVRQAPGKG |
| Y36G | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQGEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 107 | QVQLVESGGGVVQPGRSLRLDCKASGITFSNSGMHWVRQAPGKG |
| Y36H | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQHEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 108 | QVQLVESGGGVVQPGRSLRLDCKASGITFSNSGMHWVRQAPGKG |
| Y36I | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQIEELKDEATSC |
| | SLHRSAHNATHATYTCHMDVFHFMADDIFSVNITDQSGNYSQECG |
| | SFLLAESIKPAPPFNVTVTFSGQYNISWRSDYEDPAFYMLKGKLQY |
| | ELQYRNRGDPWAVSPRRKLISVDSRSVSLLPLEFRKDSSYELQVRA |
| | GPMPGSSYQGTWSEWSDPVIFQTQSEELKE |
| 109 | QVQLVESGGGVVQPGRSLRLDCKASGITFSNSGMHWVRQAPGKG |
| Y36K | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQKEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 110 | QVQLVESGGGVVQPGRSLRLDCKASGITFSNSGMHWVRQAPGKG |
| Y36M | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQMEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 111 | QVQLVESGGGVVQPGRSLRLDCKASGITFSNSGMHWVRQAPGKG |
| Y36N | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQNEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 112 | QVQLVESGGGVVQPGRSLRLDCKASGITFSNSGMHWVRQAPGKG |
| Y36P | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQPEELKDEATSC |
| | SLHRSAHNATHATYTCHMDVFHFMADDIFSVNITDQSGNYSQECG |
| | SFLLAESIKPAPPFNVTVTFSGQYNISWRSDYEDPAFYMLKGKLQY |
| | ELQYRNRGDPWAVSPRRKLISVDSRSVSLLPLEFRKDSSYELQVRA |
| | GPMPGSSYQGTWSEWSDPVIFQTQSEELKE |
| 113 | QVQLVESGGGVVQPGRSLRLDCKASGITFSNSGMHWVRQAPGKG |
| Y36Q | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQQEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 114 | QVQLVESGGGVVQPGRSLRLDCKASGITFSNSGMHWVRQAPGKG |
| Y36R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQREELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 115 | QVQLVESGGGVVQPGRSLRLDCKASGITFSNSGMHWVRQAPGKG |
| Y36S | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQSEELKDEATSC |
| | SLHRSAHNATHATYTCHMDVFHFMADDIFSVNITDQSGNYSQECG |
| | SFLLAESIKPAPPFNVTVTFSGQYNISWRSDYEDPAFYMLKGKLQY |
| | ELQYRNRGDPWAVSPRRKLISVDSRSVSLLPLEFRKDSSYELQVRA |
| | GPMPGSSYQGTWSEWSDPVIFQTQSEELKE |
| 116 | QVQLVESGGGVVQPGRSLRLDCKASGITFSNSGMHWVRQAPGKG |
| Y36T | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQTEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 117 | QVQLVESGGGVVQPGRSLRLDCKASGITFSNSGMHWVRQAPGKG |
| Y36V | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQVEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 118 | QVQLVESGGGVVQPGRSLRLDCKASGITFSNSGMHWVRQAPGKG |
| E38C | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYECLKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 119 | QVQLVESGGGVVQPGRSLRLDCKASGITFSNSGMHWVRQAPGKG |
| E38Y | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEYLKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 120 | QVQLVESGGGVVQPGRSLRLDCKASGITFSNSGMHWVRQAPGKG |
| L39C | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEECKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 121 | QVQLVESGGGVVQPGRSLRLDCKASGITFSNSGMHWVRQAPGKG |
| L39E | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEEEKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 122 | QVQLVESGGGVVQPGRSLRLDCKASGITFSNSGMHWVRQAPGKG |
| L39H | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEEHKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 123 | QVQLVESGGGVVQPGRSLRLDCKASGITFSNSGMHWVRQAPGKG |
| L39K | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEEKKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 124 | QVQLVESGGGVVQPGRSLRLDCKASGITFSNSGMHWVRQAPGKG |
| L39R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEERKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 125 | QVQLVESGGGVVQPGRSLRLDCKASGITFSNSGMHWVRQAPGKG |
| L39W | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEEWKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 126 | QVQLVESGGGVVQPGRSLRLDCKASGITFSNSGMHWVRQAPGKG |
| L39Y | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEEYKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 127 | QVQLVESGGGVVQPGRSLRLDCKASGITFSNSGMHWVRQAPGKG |
| M70F | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFFADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 128 | QVQLVESGGGVVQPGRSLRLDCKASGITFSNSGMHWVRQAPGKG |
| M70H | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFHADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 129 | QVQLVESGGGVVQPGRSLRLDCKASGITFSNSGMHWVRQAPGKG |
| M70N | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFNADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 130 | QVQLVESGGGVVQPGRSLRLDCKASGITFSNSGMHWVRQAPGKG |
| M70Q | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFQADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 131 | QVQLVESGGGVVQPGRSLRLDCKASGITFSNSGMHWVRQAPGKG |
| M70S | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFSADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 132 | QVQLVESGGGVVQPGRSLRLDCKASGITFSNSGMHWVRQAPGKG |
| M70T | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFTADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 133 | QVQLVESGGGVVQPGRSLRLDCKASGITFSNSGMHWVRQAPGKG |
| M70V | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFVADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 134 | QVQLVESGGGVVQPGRSLRLDCKASGITFSNSGMHWVRQAPGKG |
| M70W | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFWADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 135 | QVQLVESGGGVVQPGRSLRLDCKASGITFSNSGMHWVRQAPGKG |
| M70Y | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFYADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 136 | QVQLVESGGGVVQPGRSLRLDCKASGITFSNSGMHWVRQAPGKG |
| A71E | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMEDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 137 | QVQLVESGGGVVQPGRSLRLDCKASGITFSNSGMHWVRQAPGKG |
| A71F | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMFDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 138 | QVQLVESGGGVVQPGRSLRLDCKASGITFSNSGMHWVRQAPGKG |
| A71I | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMIDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 139 | QVQLVESGGGVVQPGRSLRLDCKASGITFSNSGMHWVRQAPGKG |
| A71L | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMLDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 140 | QVQLVESGGGVVQPGRSLRLDCKASGITFSNSGMHWVRQAPGKG |
| A71Q | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMQDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 141 | QVQLVESGGGVVQPGRSLRLDCKASGITFSNSGMHWVRQAPGKG |
| A71R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMRDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 142 | QVQLVESGGGVVQPGRSLRLDCKASGITFSNSGMHWVRQAPGKG |
| A71W | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMWDDIFSVNITDQSGNYSQE |
| | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 143 | QVQLVESGGGVVQPGRSLRLDCKASGITFSNSGMHWVRQAPGKG |
| A71Y | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMYDDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 144 | QVQLVESGGGVVQPGRSLRLDCKASGITFSNSGMHWVRQAPGKG |
| I74R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDRFSVNITDQSGNYSQE |
| | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 145 | QVQLVESGGGVVQPGRSLRLDCKASGITFSNSGMHWVRQAPGKG |
| I74W | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDWFSVNITDQSGNYSQE |
| | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 146 | QVQLVESGGGVVQPGRSLRLDCKASGITFSNSGMHWVRQAPGKG |
| L94F | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDIFSVNITDQSGNYSQEC |
| | GSFFLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 147 | QVQLVESGGGVVQPGRSLRLDCKASGITFSNSGMHWVRQAPGKG |
| L94K | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDIFSVNITDQSGNYSQEC |
| | GSFKLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 148 | QVQLVESGGGVVQPGRSLRLDCKASGITFSNSGMHWVRQAPGKG |
| L94Q | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDIFSVNITDQSGNYSQEC |
| | GSFQLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 149 | QVQLVESGGGVVQPGRSLRLDCKASGITFSNSGMHWVRQAPGKG |
| L94R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDIFSVNITDQSGNYSQEC |
| | GSFRLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 150 | QVQLVESGGGVVQPGRSLRLDCKASGITFSNSGMHWVRQAPGKG |
| L94Y | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDIFSVNITDQSGNYSQEC |
| | GSFYLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 151 (Ipilimumab | QVQLVESGGGVVQPGRSLRLSCAASGFTFSSYTMHWVRQAPGKG |
| heavy chain) | LEWVTFISYDGNNKYY |
| | ADSVKGRFTISRDNSKNTLYLQMNSLRAEDTAIYYCARTGWLGPF |
| | DYWGQGTLVTVSSAS |
| | TKGPSVFPLAPSSKSTSGGTAALGCLVKDYFPEPVTVSWNSGALTS |
| | GVHTFPAVLQSSGL |
| | YSLSSWTVPSSSLGTQTYICNVNHKPSNTKVDKRVEPKSCDKTHT |
| | CPPCPAPELLGGPS |
| | VFLFPPKPKDTLMISRTPEVTCVVVDVSHEDPEVKFNWYVDGVEV |
| | HNAKTKPREEQYNST |
| | YRVVSVLTVLHQDWLNGKEYKCKVSNKALPAPIEKTISKAKGQPR |
| | EPQVYTLPPSRDELT |
| | KNQVSLTCLVKGFYPSDIAVEWESNGQPENNYKTTPPVLDSDGSFF |
| | LYSKLTVDKSRWQQ |
| | GNVFSCSVMHEALHNHYTQKSLSLSPGK |
| 152 (Ipilimumab | EIVLTQSPGTLSLSPGERATLSCRASQSVGSSYLAWYQQKPGQAPR |
| light chain) | LLIYGAFSRATGIP |
| | DRFSGSGSGTDFTLTISRLEPEDFAVYYCQQYGSSPWTFGQGTKVEI |
| | KRTVAAPSVFIFP |
| | PSDEQLKSGTASVVCLLNNFYPREAKVQWKVDNALQSGNSQESV |
| | TEQDSKDSTYSLSSTL |
| | TLSKADYEKHKVYACEVTHQGLSSPVTKSFNRGEC |
| 153 | QVQLVESGGG VVQPGRSLRL SCAASGFTFS SYGMHWVRQA |
| (tremelimumab | PGKGLEWVAV IWYDGSNKYY |
| heavy chain) | ADSVKGRFTI SRDNSKNTLY LQMNSLRAED TAVYYCARDP |
| | RGATLYYYYY GMDVWGQGTT |
| | VTVSSASTKG PSVFPLAPCS RSTSESTAAL GCLVKDYFPE |
| | PVTVSWNSGA LTSGVHTFPA |
| | VLQSSGLYSL SSVVTVPSSN FGTQTYTCNV DHKPSNTKVD |
| | KTVERKCCVE CPPCPAPPVA |
| | GPSVFLFPPK PKDTLMISRT PEVTCVVVDV SHEDPEVQFN |
| | WYVDGVEVHN AKTKPREEQF |
| | NSTFRVVSVL TVVHQDWLNG KEYKCKVSNK GLPAPIEKTI |
| | SKTKGQPREP QVYTLPPSRE |
| | EMTKNQVSLT CLVKGFYPSD IAVEWESNGQ PENNYKTTPP |
| | MLDSDGSFFL YSKLTVDKSR |
| | WQQGNVFSCS VMHEALHNHY TQKSLSLSPG K |
| 154 | DIQMTQSPSS LSASVGDRVT ITCRASQSIN SYLDWYQQKP |
| (tremelimumab | GKAPKLLIYA ASSLQSGVPS |
| light chain) | RFSGSGSGTD FTLTISSLQP EDFATYYCQQ YYSTPFTFGP |
| | GTKVEIKRTV AAPSVFIFPP |
| | SDEQLKSGTA SVVCLLNNFY PREAKVQWKV DNALQSGNSQ |
| | ESVTEQDSKD STYSLSSTLT |
| | LSKADYEKHK VYACEVTHQG LSSPVTKSFN RGEC |
| 155 | CPDLVCYTDALQTVICILEMWNLHPSTLTLTWQDQYEELKDEATS |
| Y10A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 156 | CPDLVCYTDYLQTVICILEMWNLHPSTLTLTWQDQAEELKDEATS |
| Y36A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 157 | CPDLVCYTDYLQTVICILEMWNLHPSTLTLTWQDQYEALKDEATS |
| E38A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 158 | CPDLVCYTDYLQTVICILEMWNLHPSTLTLTWQDQYEEAKDEATS |
| L39A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 159 | CPDLVCYTDYLQTVICILEMWNLHPSTLTLTWQDQYEELKDEATS |
| F67A | CSLHRSAHNATHATYTCHMDVAHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 160 | CPDLVCYTDYLQTVICILEMWNLHPSTLTLTWQDQYEELKDEATS |
| H68A | CSLHRSAHNATHATYTCHMDVFAFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 161 | CPDLVCYTDYLQTVICILEMWNLHPSTLTLTWQDQYEELKDEATS |
| I74A | CSLHRSAHNATHATYTCHMDVFHFMADDAFSVNITDQSGNYSQE |
| (IL-21Rα mutein) | CGSFLLAESIKPAPPFNVTVTFSGQYNISWRSDYEDPAFYMLKGKL |
| | QYELQYRNRGDPWAVSPRRKLISVDSRSVSLLPLEFRKDSSYELQV |
| | RAGPMPGSSYQGTWSEWSDPVIFQTQSEELKE |
| 162 | CPDLVCYTDYLQTVICILEMWNLHPSTLTLTWQDQYEELKDEATS |
| L94A | CSLHRSAHNATHATYTCHMDVFHFMADDIFSVNITDQSGNYSQEC |
| (IL-21Rα mutein) | GSFALAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 163 | CPDLVCYTDYLQTVICILEMWNLHPSTLTLTWQDQYEELKDEATS |
| P126A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDAAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 164 | CPDLVCYTDYLQTVICILEMWNLHPSTLTLTWQDQYEELKDEATS |
| Y129A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFAMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 165 | CPDLVCYTDYLQTVICILEMWNLHPSTLTLTWQDQYEELKDEATS |
| M130A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYALKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 166 | CPDLVCYTDYLQTVICILEMWNLHPSTLTLTWQDQYEELKDEATS |
| K134A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGALQ |
| | YELQYRNRGDPWAVSPRRKLISVDSRSVSLLPLEFRKDSSYELQVR |
| | AGPMPGSSYQGTWSEWSDPVIFQTQSEELKE |
| 167 | CPDLVCYTDYLQTVICILEMWNLHPSTLTLTWQDQYEELKDEATS |
| S189A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGASYQGTWSEWSDPVIFQTQSEELKE |
| 168 | CPDLVCYTDYLQTVICILEMWNLHPSTLTLTWQDQYEELKDEATS |
| S190A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSAYQGTWSEWSDPVIFQTQSEELKE |
| 169 | CPDLVCYTDYLQTVICILEMWNLHPSTLTLTWQDQYEELKDEATS |
| Y191A | CSLHRSAHNATHATYTCHMDVFHFMADDIFSVNITDQSGNYSQEC |
| (IL-21Rα mutein) | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSAQGTWSEWSDPVIFQTQSEELKE |
| 170 | PKSCDKTHTCPPCPAPELLGGPSVFLFPPKPKDTLMISRTPEVTCVV |
| Y10A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDALQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 171 | PKSCDKTHTCPPCPAPELLGGPSVFLFPPKPKDTLMISRTPEVTCVV |
| Y36A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQAEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 172 | PKSCDKTHTCPPCPAPELLGGPSVFLFPPKPKDTLMISRTPEVTCVV |
| E38A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEALKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 173 | PKSCDKTHTCPPCPAPELLGGPSVFLFPPKPKDTLMISRTPEVTCVV |
| L39A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEEAKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 174 | PKSCDKTHTCPPCPAPELLGGPSVFLFPPKPKDTLMISRTPEVTCVV |
| F67A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVAHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 175 | PKSCDKTHTCPPCPAPELLGGPSVFLFPPKPKDTLMISRTPEVTCVV |
| H68A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFAFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 176 | PKSCDKTHTCPPCPAPELLGGPSVFLFPPKPKDTLMISRTPEVTCVV |
| I74A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDAFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 177 | PKSCDKTHTCPPCPAPELLGGPSVFLFPPKPKDTLMISRTPEVTCVV |
| L94A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21 Ra mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFALAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 178 | PKSCDKTHTCPPCPAPELLGGPSVFLFPPKPKDTLMISRTPEVTCVV |
| P126A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDAAFYMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 179 | PKSCDKTHTCPPCPAPELLGGPSVFLFPPKPKDTLMISRTPEVTCVV |
| Y129A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFAMLKGKLQYELQYRNRGDPWAVSPRRKLISVDS |
| | RSVSLLPLEFRKDSSYELQVRAGPMPGSSYQGTWSEWSDPVIFQT |
| | QSEELKE |
| 180 | PKSCDKTHTCPPCPAPELLGGPSVFLFPPKPKDTLMISRTPEVTCVV |
| M130A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYALKGKLQYELQYRNRGDPWAVSPRRKLISVDSR |
| | SVSLLPLEFRKDSSYELQVRAGPMPGSSYQGTWSEWSDPVIFQTQ |
| | SEELKE |
| 181 | PKSCDKTHTCPPCPAPELLGGPSVFLFPPKPKDTLMISRTPEVTCVV |
| K134A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGALQYELQYRNRGDPWAVSPRRKLISVDS |
| | RSVSLLPLEFRKDSSYELQVRAGPMPGSSYQGTWSEWSDPVIFQT |
| | QSEELKE |
| 182 | PKSCDKTHTCPPCPAPELLGGPSVFLFPPKPKDTLMISRTPEVTCVV |
| S189A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGASYQGTWSEWSDPVIFQT |
| | QSEELKE |
| 183 | PKSCDKTHTCPPCPAPELLGGPSVFLFPPKPKDTLMISRTPEVTCVV |
| S190A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSAYQGTWSEWSDPVIFQT |
| | QSEELKE |
| 184 | PKSCDKTHTCPPCPAPELLGGPSVFLFPPKPKDTLMISRTPEVTCVV |
| Y191A | VDVSHEDPEVKFNWYVDGVEVHNAKTKPREEQYNSTYRVVSVLT |
| (Fc-linker-IL- | VLHQDWLNGKEYKCKVSNKALPAPIEKTISKAKGQPREPQVYTLP |
| 21Rα mutein) | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGKGGGGSGGGGSGGGGSCPDLVCYTDYLQTVICILEMWNLHPS |
| | TLTLTWQDQYEELKDEATSCSLHRSAHNATHATYTCHMDVFHFM |
| | ADDIFSVNITDQSGNYSQECGSFLLAESIKPAPPFNVTVTFSGQYNI |
| | SWRSDYEDPAFYMLKGKLQYELQYRNRGDPWAVSPRRKLISVDS |
| | RSVSLLPLEFRKDSSYELQVRAGPMPGSSAQGTWSEWSDPVIFQT |
| | QSEELKE |
| 185 | PKSCDKTHTCPPCPAPELLGGPSVFLFPPKPKDTLMISRTPEVTCVV |
| IgG1 Fc moiety | VDVSHEDPEVKFNWYVDGVEVHNAKTKPREEQYNSTYRVVSVLT |
| (WT) | VLHQDWLNGKEYKCKVSNKALPAPIEKTISKAKGQPREPQVYTLP |
| | PSRDELTKNQVSLTCLVKGFYPSDIAVEWESNGQPENNYKTTPPVL |
| | DSDGSFFLYSKLTVDKSRWQQGNVFSCSVMHEALHNHYTQKSLSL |
| | SPGK |
| 186 | ERKCCVECPPCPAPPVAGPSVFLFPPKPKDTLMISRTPEVTCVVVD |
| IgG2 Fc moiety | VSHEDPEVQFNWYVDGVEVHNAKTKPREEQFNSTFRVVSVLTVV |
| (WT) | HQDWLNGKEYKCKVSNKGLPAPIEKTISKTKGQPREPQVYTLPPS |
| | REEMTKNQVSLTCLVKGFYPSDISVEWESNGQPENNYKTTPPMLD |
| | SDGSFFLYSKLTVDKSRWQQGNVFSCSVMHEALHNHYTQKSL |
| | SLSPGK |
| 187 | ELKTPLGDTTHTCPRCPEPKSCDTPPPCPRCPEPKSCDTPPPCPRCP |
| IgG3 Fc moiety | EPKSCDTPPPCPRCPAPELLGGPSVFLFPPKPKDTLMISRTPEVTCV |
| (WT) | VVDVSHEDPEVQFKWYVDGVEVHNAKTKPREEQYNSTFRVVSVL |
| | TVLHQDWLNGKEYKCKVSNKALPAPIEKTISKTKGQPREPQVYTL |
| | PPSREEMTKNQVSLTCLVKGFYPSDIAVEWESSGQPENNYNTTPPM |
| | LDSDGSFFLYSKLTVDKSRWQQGNIFSCSVMHEALHNRFTQKSLSL |
| | SPGK |
| 188 | ESKYGPPCPSCPAPEFLGGPSVFLFPPKPKDTLMISRTPEVTCVVVD |
| IgG4 Fc moiety | VSQEDPEVQFNWYVDGVEVHNAKTKPREEQFNSTYRVVSVLTVL |
| (WT) | HQDWLNGKEYKCKVSNKGLPSSIEKTISKAKGQPREPQVYTLPPS |
| | QEEMTKNQVSLTCLVKGFYPSDIAVEWESNGQPENNYKTTPPVLD |
| | SDGSFFLYSRLTVDKSRWQEGNVFSCSVMHEALHNHYTQKSLSLS |
| | LGK |
| 189 | ASTKGPSVFPLAPSSKSTSGGTAALGCLVKDYFPEPVTVSWNSGAL |
| IGHG1 | TSGVHTFPAVLQSSGLYSLSSVVTVPSSSLGTQTYICNVNHKPSNTK |
| (Immunoglobulin | VDKKVEPKSCDKTHTCPPCPAPEAAGGPSVFLFPPKPKDTLMISRT |
| heavy constant | PEVTCVVVDVSHEDPEVKFNWYVDGVEVHNAKTKPREEQYNST |
| gamma 1) with | YRVVSVLTVLHQDWLNGKEYKCKVSNKALPAPIEKTISKAKGQPR |
| ‘LALA’(L234A/ | EPQVYTLPPSRDELTKNQVSLTCLVKGFYPSDIAVEWESNGQPENN |
| L235A) mutation | YKTTPPVLDSDGSFFLYSKLTVDKSRWQQGNVFSCSVMHEALHNH |
| | YTQKSLSLSPGK |
| 190 | ASTKGPSVFPLAPCSRSTSESTAALGCLVKDYFPEPVTVSWNSGAL |
| IGHG4 | TSGVHTFPAVLQSSGLYSLSSVVTVPSSSLGTKTYTCNVDHKPSNT |
| (Immunoglobulin | KVDKRVESKYGPPCPPCPAPEFEGGPSVFLFPPKPKDTLMISRTPEV |
| heavy constant | TCVVVDVSQEDPEVQFNWYVDGVEVHNAKTKPREEQFNSTYRV |
| gamma 4) with | VSVLTVLHQDWLNGKEYKCKVSNKGLPSSIEKTISKAKGQPREPQ |
| SPLE‘(S228P/L | VYTLPPSQEEMTKNQVSLTCLVKGFYPSDIAVEWESNGQPENNYK |
| 235E) mutation | TTPPVLDSDGSFFLYSRLTVDKSRWQEGNVFSCSVMHEALHNHYT |
| | QKSLSLSLGK |
| 191 | QVQLVESGGGVVQPGRSLRLDCKASGITFSNSGMHWVRQAPGKG |
| αPD-1-linker- | LEWVAVIWYDGSKRYYADSVKGRFTISRDNSKNTLFLQMNSLRAE |
| IL21RαWT | DTAVYYCATNDDYWGQGTLVTVSSASTKGPSVFPLAPCSRSTSEST |
| | AALGCLVKDYFPEPVTVSWNSGALTSGVHTFPAVLQSSGLYSLSSV |
| | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGS |
| | CPDLVCYTDYLQTVICILEMWNLHPSTLTLTWQDQYEELKDEATS |
| | CSLHRSAHNATHATYTCHMDVFHFMADDIFSVNITDQSGNYSQEC |
| | GSFLLAESIKPAPPFNVTVTFSGQYNISWRSDYEDPAFYMLKGKLQ |
| | YELQYRNRGDPWAVSPRRKLISVDSRSVSLLPLEFRKDSSYELQVR |
| | AGPMPGSSYQGTWSEWSDPVIFQTQSEELKE |
| 192 | QVQLVESGGGVVQPGRSLRLDCKASGITFSNSGMHWVRQAPG |
| Y36A + D72E | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQAEELKDEATSCSLHRSAHNATH |
| | ATYTCHMDVFHFMAEDIFSVNITDQSGNYSQECGSFLLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 193 | QVQLVESGGGVVQPGRSLRLDCKASGITFSNSGMHWVRQAPG |
| Y36A + L94R | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQAEELKDEATSCSLHRSAHNATH |
| | ATYTCHMDVFHFMADDIFSVNITDQSGNYSQECGSFRLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 194 | QVQLVESGGGVVQPGRSLRLDCKASGITFSNSGMHWVRQAPG |
| E38A + D72E | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQYEALKDEATSCSLHRSAHNATH |
| | ATYTCHMDVFHFMAEDIFSVNITDQSGNYSQECGSFLLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 195 | QVQLVESGGGVVQPGRSLRLDCKASGITFSNSGMHWVRQAPG |
| E38A + L94K | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQYEALKDEATSCSLHRSAHNATH |
| | ATYTCHMDVFHFMADDIFSVNITDQSGNYSQECGSFKLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 196 | QVQLVESGGGVVQPGRSLRLDCKASGITFSNSGMHWVRQAPG |
| E38A + L94R | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQYEALKDEATSCSLHRSAHNATH |
| | ATYTCHMDVFHFMADDIFSVNITDQSGNYSQECGSFRLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 197 | QVQLVESGGGVVQPGRSLRLDCKASGITFSNSGMHWVRQAPG |
| E38R + D72R | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQYERLKDEATSCSLHRSAHNATH |
| | ATYTCHMDVFHFMARDIFSVNITDQSGNYSQECGSFLLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 198 | QVQLVESGGGVVQPGRSLRLDCKASGITFSNSGMHWVRQAPG |
| D72E + L94K | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQYEELKDEATSCSLHRSAHNATH |
| | ATYTCHMDVFHFMAEDIFSVNITDQSGNYSQECGSFKLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 199 | QVQLVESGGGVVQPGRSLRLDCKASGITFSNSGMHWVRQAPG |
| D72E + L94R | KGLEWVAVIWYDGSKRYYADSVKGRFTISRDNSKNTLFLQMN |
| (αPD- | SLRAEDTAVYYCATNDDYWGQGTLVTVSSASTKGPSVFPLAP |
| 1 + linker + IL21Rα | CSRSTSESTAALGCLVKDYFPEPVTVSWNSGALTSGVHTFPAVL |
| Mutein) | QSSGLYSLSSVVTVPSSSLGTKTYTCNVDHKPSNTKVDKRVES |
| | KYGPPCPPCPAPEFLGGPSVFLFPPKPKDTLMISRTPEVTCVVV |
| | DVSQEDPEVQFNWYVDGVEVHNAKTKPREEQFNSTYRVVSV |
| | LTVLHQDWLNGKEYKCKVSNKGLPSSIEKTISKAKGQPREPQV |
| | YTLPPSQEEMTKNQVSLSCAVKGFYPSDIAVEWESNGQPENNY |
| | KTTPPVLDSDGSFFLVSRLTVDKSRWQEGNVFSCSVMHEALH |
| | NHYTQKSLSLSLGGGGGSGGGGSGGGGSCPDLVCYTDYLQTV |
| | ICILEMWNLHPSTLTLTWQDQYEELKDEATSCSLHRSAHNATH |
| | ATYTCHMDVFHFMAEDIFSVNITDQSGNYSQECGSFRLAESIK |
| | PAPPFNVTVTFSGQYNISWRSDYEDPAFYMLKGKLQYELQYR |
| | NRGDPWAVSPRRKLISVDSRSVSLLPLEFRKDSSYELQVRAGP |
| | MPGSSYQGTWSEWSDPVIFQTQSEELKE |
| 200 | QVQLVESGGGVVQPGRSLRLDCKASGITFSNSGMHWVRQAPGKG |
| E38R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYERLKDE |
| | ATSCSLHRSAHNATHATYTCHMDVFHFMADDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 201 | QVQLVESGGGVVQPGRSLRLDCKASGITFSNSGMHWVRQAPGKG |
| M70C | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFCADDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 202 | QVQLVESGGGVVQPGRSLRLDCKASGITFSNSGMHWVRQAPGKG |
| M70D | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFDADDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 203 | QVQLVESGGGVVQPGRSLRLDCKASGITFSNSGMHWVRQAPGKG |
| M70G | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFGADDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 204 | QVQLVESGGGVVQPGRSLRLDCKASGITFSNSGMHWVRQAPGKG |
| M70R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFRADDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 205 | QVQLVESGGGVVQPGRSLRLDCKASGITFSNSGMHWVRQAPGKG |
| D72A | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFMAADIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 206 | QVQLVESGGGVVQPGRSLRLDCKASGITFSNSGMHWVRQAPGKG |
| D72E | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFMAEDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 207 | QVQLVESGGGVVQPGRSLRLDCKASGITFSNSGMHWVRQAPGKG |
| D72Q | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFMAQDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 208 | QVQLVESGGGVVQPGRSLRLDCKASGITFSNSGMHWVRQAPGKG |
| D72R | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFMARDIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 209 | QVQLVESGGGVVQPGRSLRLDCKASGITFSNSGMHWVRQAPGKG |
| D73A | LEWVAVIWYDGSKRYYADSVKGRFTISRDNSKNTLFLQMNSLRAE |
| (αPD- | DTAVYYCATNDDYWGQGTLVTVSSASTKGPSVFPLAPCSRSTSEST |
| 1 + linker + IL21Rα | AALGCLVKDYFPEPVTVSWNSGALTSGVHTFPAVLQSSGLYSLSSV |
| Mutein) | VTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYV |
| | DGVEVHNAKTKPREEQFNSTYRVVSVLTVLHQDWLNGKEYKCK |
| | VSNKGLPSSIEKTISKAKGQPREPQVYTLPPSQEEMTKNQVSLSCA |
| | VKGFYPSDIAVEWESNGQPENNYKTTPPVLDSDGSFFLVSRLTVDK |
| | SRWQEGNVFSCSVMHEALHNHYTQKSLSLSLGGGGGSGGGGSGG |
| | GGSCPDLVCYTDYLQTVICILEMWNLHPSTLTLTWQDQYEELKDE |
| | ATSCSLHRSAHNATHATYTCHMDVFHFMADAIFSVNITDQSGNYS |
| | QECGSFLLAESIKPAPPFNVTVTFSGQYNISWRSDYEDPAFYMLKG |
| | KLQYELQYRNRGDPWAVSPRRKLISVDSRSVSLLPLEFRKDSSYEL |
| | QVRAGPMPGSSYQGTWSEWSDPVIFQTQSEELKE |
| 210 | CCGAAATCATGTGACAAAACTCATACTTGTCCTCCATGCCCA |
| (Polynucleotide | GCCCCAGAATTGCTGGGGGGACCATCTGTGTTCCTTTTCCCC |
| encoding IgG1 | CCTAAGCCAAAAGACACTCTGATGATCAGTCGCACTCCTGA |
| Fc-IL21Rα | AGTGACCTGCGTCGTGGTAGACGTCTCTCACGAAGATCCCG |
| (wildtype)) | AGGTCAAATTTAACTGGTATGTGGATGGCGTGGAAGTTCATA |
| | ACGCAAAAACCAAACCCCGCGAAGAACAATATAATAGCACA |
| | TACCGTGTTGTTAGCGTTTTGACAGTCCTTCACCAGGATTGG |
| | CTCAACGGAAAAGAGTACAAGTGCAAGGTGTCCAATAAAG |
| | CATTGCCCGCCCCTATAGAGAAGACTATTAGCAAGGCCAAA |
| | GGTCAGCCCCGGGAGCCTCAGGTGTATACATTGCCTCCCAG |
| | CCGCGATGAACTCACTAAAAACCAAGTCAGCCTCACATGTC |
| | TGGTTAAAGGTTTTTACCCCAGCGATATCGCAGTCGAGTGGG |
| | AATCTAATGGGCAGCCTGAAAATAACTATAAGACAACCCCA |
| | CCAGTGTTGGATAGCGATGGCAGCTTTTTTCTTTACTCTAAG |
| | TTGACTGTTGACAAGAGCAGGTGGCAACAAGGCAACGTGT |
| | TTAGCTGCAGTGTCATGCACGAAGCACTCCACAATCATTACA |
| | CCCAGAAGAGTCTGAGCTTGTCACCTGGAAAGGGTGGAGG |
| | CGGTTCAGGCGGAGGTGGCTCTGGCGGTGGCGGATCATGTC |
| | CTGACCTGGTGTGCTACACCGACTACCTGCAGACCGTGATC |
| | TGCATCCTGGAGATGTGGAACCTGCATCCTTCTACCCTGACA |
| | CTGACCTGGCAGGACCAGTACGAGGAACTGAAGGACGAGG |
| | CCACCTCCTGCTCCCTGCACAGATCTGCTCACAACGCCACC |
| | CACGCTACCTACACCTGTCACATGGACGTGTTCCACTTCATG |
| | GCCGACGACATCTTTTCTGTGAACATCACCGATCAGTCTGGC |
| | AACTACTCCCAAGAGTGCGGCTCTTTCCTGCTGGCCGAGTC |
| | CATCAAGCCTGCTCCTCCTTTCAACGTGACCGTGACCTTCTC |
| | CGGCCAGTACAACATCTCTTGGCGGTCCGACTACGAGGACC |
| | CCGCCTTCTACATGCTGAAGGGCAAGCTGCAGTACGAGCTG |
| | CAGTACCGGAACAGAGGCGACCCTTGGGCCGTGTCCCCTAG |
| | AAGAAAGCTGATCTCCGTGGACTCCAGATCCGTGTCTCTGC |
| | TGCCTCTGGAATTCCGGAAGGACTCTAGCTACGAACTGCAA |
| | GTGCGGGCTGGCCCTATGCCTGGCTCCTCCTACCAGGGAAC |
| | ATGGTCCGAGTGGAGCGATCCTGTGATCTTCCAGACCCAGT |
| | CCGAAGAGCTGAAAGAG |
| 211 | CAGGTGCAGCTGGTGGAGTCCGGAGGAGGAGTGGTGCAG |
| (Polynucleotide | CCAGGCAGGTCCCTGCGGCTGGACTGTAAGGCCTCCGGCA |
| encoding αPD-1 | TCACCTTTTCTAACTCCGGAATGCATTGGGTGAGGCAGGCT |
| IL21RαWT) | CCAGGCAAGGGCCTGGAGTGGGTGGCTGTGATCTGGTACG |
| | ACGGCAGCAAGCGGTACTATGCCGATTCTGTGAAGGGCAG |
| | ATTCACAATCTCTCGCGACAACTCCAAGAATACCCTGTTTC |
| | TGCAGATGAACTCTCTGAGGGCCGAGGATACAGCCGTGTA |
| | CTATTGCGCTACCAATGACGATTACTGGGGCCAGGGCACAC |
| | TGGTGACCGTGTCCAGCGCCAGCACAAAGGGACCATCCGT |
| | GTTCCCACTGGCTCCATGCAGCCGGTCTACATCCGAGAGCA |
| | CCGCCGCTCTGGGATGTCTGGTGAAGGATTATTTCCCTGAG |
| | CCAGTGACCGTGAGCTGGAACTCCGGCGCCCTGACATCTG |
| | GCGTGCACACCTTTCCTGCTGTGCTGCAGTCTTCCGGCCTG |
| | TACTCCCTGAGCTCTGTGGTGACAGTGCCCTCCAGCTCTCT |
| | GGGCACCAAGACATATACCTGCAACGTGGACCATAAGCCTT |
| | CCAATACCAAGGTGGATAAGAGAGTGGAGAGCAAGTACGG |
| | ACCACCTTGCCCACCATGTCCAGCTCCTGAGTTTCTGGGAG |
| | GACCATCCGTGTTCCTGTTTCCTCCAAAGCCTAAGGACACC |
| | CTGATGATCAGCCGCACACCTGAGGTGACCTGCGTGGTGG |
| | TGGACGTGTCTCAGGAGGACCCCGAGGTGCAGTTCAACTG |
| | GTACGTGGATGGCGTGGAGGTGCACAATGCTAAGACCAAG |
| | CCTAGAGAGGAGCAGTTTAACTCCACATACCGCGTGGTGA |
| | GCGTGCTGACCGTGCTGCATCAGGACTGGCTGAACGGCAA |
| | GGAGTATAAGTGCAAGGTGTCCAATAAGGGCCTGCCATCCA |
| | GCATCGAGAAGACAATCAGCAAGGCCAAGGGCCAGCCTAG |
| | GGAGCCACAGGTGTACACCCTGCCCCCTTCTCAGGAGGAG |
| | ATGACAAAGAACCAGGTGTCCCTGTCCTGTGCCGTGAAGG |
| | GCTTCTATCCAAGCGACATCGCTGTGGAGTGGGAGTCTAAT |
| | GGCCAGCCCGAGAACAATTACAAGACCACACCACCCGTGC |
| | TGGACTCCGATGGCAGCTTCTTTCTGGTCTCCAGGCTGACA |
| | GTGGATAAGAGCCGGTGGCAGGAGGGCAACGTGTTTTCTT |
| | GTTCCGTGATGCACGAGGCTCTGCACAATCATTACACCCAG |
| | AAGAGCCTGTCTCTGTCCCTGGGCGGTGGCGGTGGCTCTG |
| | GCGGAGGTGGCTCAGGTGGCGGCGGATCCTGTCCTGATCT |
| | CGTGTGCTATACCGACTACCTCCAGACCGTTATTTGTATCCT |
| | TGAGATGTGGAATTTGCACCCATCAACACTGACTCTGACTT |
| | GGCAGGATCAATACGAGGAGCTGAAAGACGAGGCCACATC |
| | CTGCTCCTTGCATCGATCAGCACACAACGCCACTCATGCAA |
| | CATACACTTGCCATATGGATGTGTTCCACTTCATGGCAGATG |
| | ATATTTTTTCAGTTAACATTACAGATCAATCCGGCAACTATT |
| | CACAGGAATGTGGCTCTTTTCTTCTGGCAGAATCAATAAAG |
| | CCCGCACCTCCTTTCAACGTGACTGTCACCTTCTCAGGACA |
| | ATATAATATCAGCTGGCGATCTGACTATGAGGACCCTGCCTT |
| | TTACATGCTGAAAGGCAAGCTCCAATACGAACTTCAATATC |
| | GTAATAGGGGGGACCCATGGGCCGTCAGTCCTCGACGGAA |
| | GCTGATATCCGTGGACTCTAGAAGTGTCTCTCTCTTGCCCCT |
| | CGAATTTAGGAAAGACTCATCCTACGAGCTTCAAGTTCGGG |
| | CAGGTCCCATGCCCGGCTCAAGCTATCAGGGGACATGGAG |
| | CGAGTGGTCCGACCCAGTAATTTTCCAAACCCAAAGCGAG |
| | GAATTGAAAGAG |
| 212 | GGGGS |
| (GGGGS)₁ | |
| Flexible Linker | |
| 213 | GGGGSGGGGS |
| (GGGGS)₂ | |
| Flexible Linker | |
| 214 | GGGGSGGGGSGGGGS |
| (GGGGS)₃ | |
| Flexible Linker | |
| 215 | GGGGSGGGGSGGGGSGGGGS |
| (GGGGS)₄ | |
| Flexible Linker | |
| 216 | GGGGGG |
| (Gly)₆ | |
| Flexible Linker | |
| 217 | GGGGGGGG |
| (Gly)₈ | |
| Flexible Linker | |
| 218 | EAAAK |
| (EAAAK)₁ | |
| Rigid Linker | |
| 219 | EAAAKEAAAK |
| (EAAAK)₂ | |
| Rigid Linker | |
| 220 | EAAAKEAAAKEAAAK |
| (EAAAK)₃ | |
| Rigid Linker | |
| 221 | AEAAAKEAAAKEAAAKEAAAKALEAEAAAKEAAAKEAAA |
| A(EAAAK)₄ALE | KEAAAKA |
| A(EAAAK)₄A | |
| Rigid Linker | |
| 222 | PAPAP |
| PAPAP | |
| Rigid Linker | |
| 223 | AEAAAKEAAAKA |
| AEAAAKEAAAKA | |
| Rigid Linker | |
| 224 | (AP)n, (n = 5-15) |
| (Ala-Pro)n | |
| (10-33 aa) | |
| Rigid Linker | |
| 225 | EVQLQQSGPGLVKPSQTLSLTCAISGDSVSSNSAAWNWIRQSPSRG |
| Tiragolumab | LEWLGKTYYRFKWYSDYAVSVKGRITINPDTSKNQFSLQLNSVTP |
| Heavy chain | EDTAVFYCTRESTTYDLLAGPFDYWGQGTLVTVSSASTKGPSVFPL |
| | APSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 226 | DIVMTQSPDSLAVSLGERATINCKSSQTVLYSSNNKKYLAWYQQK |
| Tiragolumab light | PGQPPNLLIYWASTRESGVPDRFSGSGSGTDFTLTISSLQAEDVAVY |
| chain | YCQQYYSTPFTFGPGTKVEIKRTVAAPSVFIFPPSDEQLKSGTASVV |
| | CLLNNFYPREAKVQWKVDNALQSGNSQESVTEQDSKDSTYSLSS |
| | TLTLSKADYEKHKVYACEVTHQGLSSPVTKSFNRGEC |
| 227 | QVQLQQWGAGLLKPSETLSLTCAVYGGSFSDYYWNWIRQPPGKG |
| Relatlimab Heavy | LEWIGEINHRGSTNSNPSLKSRVTLSLDTSKNQFSLKLRSVTAADT |
| chain | AVYYCAFGYSDYEYNWFDPWGQGTLVTVSSASTKGPSVFPLAPCS |
| | RSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGK |
| | EYKCKVSNKGLPSSIEKTISKAKGQPREPQVYTLPPSQEEMTKNQV |
| | SLTCLVKGFYPSDIAVEWESNGQPENNYKTTPPVLDSDGSFFLYSR |
| | LTVDKSRWQEGNVFSCSVMHEALHNHYTQKSLSLSLGK |
| 228 | EIVLTQSPATLSLSPGERATLSCRASQSISSYLAWYQQKPGQAPRLLI |
| Relatlimab Light | YDASNRATGIPARFSGSGSGTDFTLTISSLEPEDFAVYYCQQRSNWP |
| chain | LTFGQGTNLEIKRTVAAPSVFIFPPSDEQLKSGTASVVCLLNNFYPR |
| | EAKVQWKVDNALQSGNSQESVTEQDSKDSTYSLSSTLTLSKADYE |
| | KHKVYACEVTHQGLSSPVTKSFNRGEC |
| 229 | QVQLVESGGGVVQPGRSLRLSCAASGFTFSSYTMHWVRQAPGKG |
| Second Chain | LEWVTFISYDGNNKYYADSVKGRFTISRDNSKNTLYLQMNSLRAE |
| (Heavy chain of | DTAIYYCARTGWLGPFDYWGQGTLVTVSSASTKGPSVFPLAPSSK |
| anti-CTLA-4 | STSGGTAALGCLVKDYFPEPVTVSWNSGALTSGVHTFPAVLQSSGL |
| antibody + linker + | YSLSSWTVPSSSLGTQTYICNVNHKPSNTKVDKRVEPKSCDKTHT |
| human IL-21) | CPPCPAPELLGGPSVFLFPPKPKDTLMISRTPEVTCVVVDVSHEDPE |
| with knob | VKFNWYVDGVEVHNAKTKPREEQYNSTYRVVSVLTVLHQDWLN |
| mutation | GKEYKCKVSNKALPAPIEKTISKAKGQPREPQVYTLPPSRDELTKN |
| | QVSLWCLVKGFYPSDIAVEWESNGQPENNYKTTPPVLDSDGSFFL |
| | YSKLTVDKSRWQQGNVFSCSVMHEALHNHYTQKSLSLSPGGGGG |
| | SGGGGSGGGGSQDRHMIRMRQLIDIVDQLKNYVNDLVPEFLPAPE |
| | DVETNCEWSAFSCFQKAQLKSANTGNNERIINVSIKKLKRKPPSTN |
| | AGRRQKHRLTCPSCDSYEKKPPKEFLERFKSLLQKMIHQHLSSRTH |
| | GSEDS |
| 230 | QVQLVESGGGVVQPGRSLRLSCAASGFTFSSYTMHWVRQAPGKG |
| αCTLA-4 + | LEWVTFISYDGNNKYYADSVKGRFTISRDNSKNTLYLQMNSLRAE |
| linker + IL21Rα | DTAIYYCARTGWLGPFDYWGQGTLVTVSSASTKGPSVFPLAPSSK |
| WT | STSGGTAALGCLVKDYFPEPVTVSWNSGALTSGVHTFPAVLQSSGL |
| | YSLSSWTVPSSSLGTQTYICNVNHKPSNTKVDKRVEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSRDELTKN |
| | QVSLSCAVKGFYPSDIAVEWESNGQPENNYKTTPPVLDSDGSFFLV |
| | SKLTVDKSRWQQGNVFSCSVMHEALHNHYTQKSLSLSPGGGGGS |
| | GGGGSGGGGSCPDLVCYTDYLQTVICILEMWNLHPSTLTLTWQDQ |
| | YEELKDEATSCSLHRSAHNATHATYTCHMDVFHFMADDIFSVNIT |
| | DQSGNYSQECGSFLLAESIKPAPPFNVTVTFSGQYNISWRSDYEDP |
| | AFYMLKGKLQYELQYRNRGDPWAVSPRRKLISVDSRSVSLLPLEF |
| | RKDSSYELQVRAGPMPGSSYQGTWSEWSDPVIFQTQSEELKE |
| 231 | QVQLVESGGGVVQPGRSLRLSCAASGFTFSSYTMHWVRQAPGKG |
| M70D | LEWVTFISYDGNNKYYADSVKGRFTISRDNSKNTLYLQMNSLRAE |
| (αCTLA-4 + | DTAIYYCARTGWLGPFDYWGQGTLVTVSSASTKGPSVFPLAPSSK |
| linker + IL21Rα | STSGGTAALGCLVKDYFPEPVTVSWNSGALTSGVHTFPAVLQSSGL |
| mutein) | YSLSSWTVPSSSLGTQTYICNVNHKPSNTKVDKRVEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSRDELTKN |
| | QVSLSCAVKGFYPSDIAVEWESNGQPENNYKTTPPVLDSDGSFFLV |
| | SKLTVDKSRWQQGNVFSCSVMHEALHNHYTQKSLSLSPGGGGGS |
| | GGGGSGGGGSCPDLVCYTDYLQTVICILEMWNLHPSTLTLTWQDQ |
| | YEELKDEATSCSLHRSAHNATHATYTCHMDVFHFDADDIFSVNIT |
| | DQSGNYSQECGSFLLAESIKPAPPFNVTVTFSGQYNISWRSDYEDP |
| | AFYMLKGKLQYELQYRNRGDPWAVSPRRKLISVDSRSVSLLPLEF |
| | RKDSSYELQVRAGPMPGSSYQGTWSEWSDPVIFQTQSEELKE |
| 232 | QVQLVESGGGVVQPGRSLRLSCAASGFTFSSYTMHWVRQAPGKG |
| D72A | LEWVTFISYDGNNKYYADSVKGRFTISRDNSKNTLYLQMNSLRAE |
| (αCTLA-4 + | DTAIYYCARTGWLGPFDYWGQGTLVTVSSASTKGPSVFPLAPSSK |
| linker + IL21Rα | STSGGTAALGCLVKDYFPEPVTVSWNSGALTSGVHTFPAVLQSSGL |
| mutein) | YSLSSWTVPSSSLGTQTYICNVNHKPSNTKVDKRVEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSRDELTKN |
| | QVSLSCAVKGFYPSDIAVEWESNGQPENNYKTTPPVLDSDGSFFLV |
| | SKLTVDKSRWQQGNVFSCSVMHEALHNHYTQKSLSLSPGGGGGS |
| | GGGGSGGGGSCPDLVCYTDYLQTVICILEMWNLHPSTLTLTWQDQ |
| | YEELKDEATSCSLHRSAHNATHATYTCHMDVFHFMAADIFSVNIT |
| | DQSGNYSQECGSFLLAESIKPAPPFNVTVTFSGQYNISWRSDYEDP |
| | AFYMLKGKLQYELQYRNRGDPWAVSPRRKLISVDSRSVSLLPLEF |
| | RKDSSYELQVRAGPMPGSSYQGTWSEWSDPVIFQTQSEELKE |
| 233 | EVQLQQSGPGLVKPSQTLSLTCAISGDSVSSNSAAWNWIRQSPSRG |
| Second Chain | LEWLGKTYYRFKWYSDYAVSVKGRITINPDTSKNQFSLQLNSVTP |
| (Heavy chain of | EDTAVFYCTRESTTYDLLAGPFDYWGQGTLVTVSSASTKGPSVFPL |
| anti-TIGIT | APSSKSTSGGTAALGCLVKDYFPEPVTVSWNSGALTSGVHTFPAVL |
| antibody + | QSSGLYSLSSVVTVPSSSLGTQTYICNVNHKPSNTKVDKKVEPKSC |
| linker + human | DKTHTCPPCPAPELLGGPSVFLFPPKPKDTLMISRTPEVTCVVVDVS |
| IL-21) with knob | HEDPEVKFNWYVDGVEVHNAKTKPREEQYNSTYRVVSVLTVLHQ |
| mutation | DWLNGKEYKCKVSNKALPAPIEKTISKAKGQPREPQVYTLPPSREE |
| | MTKNQVSLWCLVKGFYPSDIAVEWESNGQPENNYKTTPPVLDSD |
| | GSFFLYSKLTVDKSRWQQGNVFSCSVMHEALHNHYTQKSLSLSPG |
| | GGGGSGGGGSGGGGSQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRLTCPSCDSYEKKPPKEFLERFKSLLQKMIHQHL |
| | SSRTHGSEDS |
| 234 | EVQLQQSGPGLVKPSQTLSLTCAISGDSVSSNSAAWNWIRQSPSRG |
| αTIGIT + | LEWLGKTYYRFKWYSDYAVSVKGRITINPDTSKNQFSLQLNSVTP |
| linker + IL21Rα | EDTAVFYCTRESTTYDLLAGPFDYWGQGTLVTVSSASTKGPSVFPL |
| WT | APSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLSCAVKGFYPSDIAVEWESNGQPENNYKTTPPVLDSDG |
| | SFFLVSKLTVDKSRWQQGNVFSCSVMHEALHNHYTQKSLSLSPGG |
| | GGGSGGGGSGGGGSCPDLVCYTDYLQTVICILEMWNLHPSTLTLT |
| | WQDQYEELKDEATSCSLHRSAHNATHATYTCHMDVFHFMADDIF |
| | SVNITDQSGNYSQECGSFLLAESIKPAPPFNVTVTFSGQYNISWRSD |
| | YEDPAFYMLKGKLQYELQYRNRGDPWAVSPRRKLISVDSRSVSLL |
| | PLEFRKDSSYELQVRAGPMPGSSYQGTWSEWSDPVIFQTQSEELK |
| | E |
| 235 | EVQLQQSGPGLVKPSQTLSLTCAISGDSVSSNSAAWNWIRQSPSRG |
| M70D | LEWLGKTYYRFKWYSDYAVSVKGRITINPDTSKNQFSLQLNSVTP |
| (αTIGIT + | EDTAVFYCTRESTTYDLLAGPFDYWGQGTLVTVSSASTKGPSVFPL |
| linker + IL21Rα | APSSKSTSGGTAALGCLVKDYFPEPVTVSWNSGALTSGVHTFPAVL |
| Mutein) | QSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLSCAVKGFYPSDIAVEWESNGQPENNYKTTPPVLDSDG |
| | SFFLVSKLTVDKSRWQQGNVFSCSVMHEALHNHYTQKSLSLSPGG |
| | GGGSGGGGSGGGGSCPDLVCYTDYLQTVICILEMWNLHPSTLTLT |
| | WQDQYEELKDEATSCSLHRSAHNATHATYTCHMDVFHFDADDIF |
| | SVNITDQSGNYSQECGSFLLAESIKPAPPFNVTVTFSGQYNISWRSD |
| | YEDPAFYMLKGKLQYELQYRNRGDPWAVSPRRKLISVDSRSVSLL |
| | PLEFRKDSSYELQVRAGPMPGSSYQGTWSEWSDPVIFQTQSEELK |
| | E |
| 236 | EVQLQQSGPGLVKPSQTLSLTCAISGDSVSSNSAAWNWIRQSPSRG |
| D72A | LEWLGKTYYRFKWYSDYAVSVKGRITINPDTSKNQFSLQLNSVTP |
| (αTIGIT + | EDTAVFYCTRESTTYDLLAGPFDYWGQGTLVTVSSASTKGPSVFPL |
| linker + IL21Rα | APSSKSTSGGTAALGCLVKDYFPEPVTVSWNSGALTSGVHTFPAVL |
| Mutein) | QSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLSCAVKGFYPSDIAVEWESNGQPENNYKTTPPVLDSDG |
| | SFFLVSKLTVDKSRWQQGNVFSCSVMHEALHNHYTQKSLSLSPGG |
| | GGGSGGGGSGGGGSCPDLVCYTDYLQTVICILEMWNLHPSTLTLT |
| | WQDQYEELKDEATSCSLHRSAHNATHATYTCHMDVFHFMAADIF |
| | SVNITDQSGNYSQECGSFLLAESIKPAPPFNVTVTFSGQYNISWRSD |
| | YEDPAFYMLKGKLQYELQYRNRGDPWAVSPRRKLISVDSRSVSLL |
| | PLEFRKDSSYELQVRAGPMPGSSYQGTWSEWSDPVIFQTQSEELK |
| | E |
| 237 | QVQLQQWGAGLLKPSETLSLTCAVYGGSFSDYYWNWIRQPPGKG |
| Second Chain | LEWIGEINHRGSTNSNPSLKSRVTLSLDTSKNQFSLKLRSVTAADT |
| (Heavy chain of | AVYYCAFGYSDYEYNWFDPWGQGTLVTVSSASTKGPSVFPLAPCS |
| anti-LAG-3 | RSTSESTAALGCLVKDYFPEPVTVSWNSGALTSGVHTFPAVLQSSG |
| antibody + linker + | LYSLSSVVTVPSSSLGTKTYTCNVDHKPSNTKVDKRVESKYGPPCP |
| human IL-21) | PCPAPEFLGGPSVFLFPPKPKDTLMISRTPEVTCVVVDVSQEDPEVQ |
| with knob | FNWYVDGVEVHNAKTKPREEQFNSTYRVVSVLTVLHQDWLNGK |
| mutation | EYKCKVSNKGLPSSIEKTISKAKGQPREPQVYTLPPSQEEMTKNQV |
| | SLWCLVKGFYPSDIAVEWESNGQPENNYKTTPPVLDSDGSFFLYSR |
| | LTVDKSRWQEGNVFSCSVMHEALHNHYTQKSLSLSLGGGGGSGG |
| | GGSGGGGSQDRHMIRMRQLIDIVDQLKNYVNDLVPEFLPAPEDVE |
| | TNCEWSAFSCFQKAQLKSANTGNNERIINVSIKKLKRKPPSTNAGR |
| | RQKHRLTCPSCDSYEKKPPKEFLERFKSLLQKMIHQHLSSRTHGSE |
| | DS |
| 238 | QVQLQQWGAGLLKPSETLSLTCAVYGGSFSDYYWNWIRQPPGKG |
| αLAG-3 + | LEWIGEINHRGSTNSNPSLKSRVTLSLDTSKNQFSLKLRSVTAADT |
| linker + IL21Rα | AVYYCAFGYSDYEYNWFDPWGQGTLVTVSSASTKGPSVFPLAPCS |
| WT | RSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGK |
| | EYKCKVSNKGLPSSIEKTISKAKGQPREPQVYTLPPSQEEMTKNQV |
| | SLSCAVKGFYPSDIAVEWESNGQPENNYKTTPPVLDSDGSFFLVSR |
| | LTVDKSRWQEGNVFSCSVMHEALHNHYTQKSLSLSLGGGGGSGG |
| | GGSGGGGSCPDLVCYTDYLQTVICILEMWNLHPSTLTLTWQDQYE |
| | ELKDEATSCSLHRSAHNATHATYTCHMDVFHFMADDIFSVNITDQ |
| | SGNYSQECGSFLLAESIKPAPPFNVTVTFSGQYNISWRSDYEDPAFY |
| | MLKGKLQYELQYRNRGDPWAVSPRRKLISVDSRSVSLLPLEFRKD |
| | SSYELQVRAGPMPGSSYQGTWSEWSDPVIFQTQSEELKE |
| 239 | QVQLQQWGAGLLKPSETLSLTCAVYGGSFSDYYWNWIRQPPGKG |
| M70D | LEWIGEINHRGSTNSNPSLKSRVTLSLDTSKNQFSLKLRSVTAADT |
| (αLAG-3 + | AVYYCAFGYSDYEYNWFDPWGQGTLVTVSSASTKGPSVFPLAPCS |
| inker + IL21Rα | RSTSESTAALGCLVKDYFPEPVTVSWNSGALTSGVHTFPAVLQSSG |
| Mutein) | LYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGK |
| | EYKCKVSNKGLPSSIEKTISKAKGQPREPQVYTLPPSQEEMTKNQV |
| | SLSCAVKGFYPSDIAVEWESNGQPENNYKTTPPVLDSDGSFFLVSR |
| | LTVDKSRWQEGNVFSCSVMHEALHNHYTQKSLSLSLGGGGGSGG |
| | GGSGGGGSCPDLVCYTDYLQTVICILEMWNLHPSTLTLTWQDQYE |
| | ELKDEATSCSLHRSAHNATHATYTCHMDVFHFDADDIFSVNITDQS |
| | GNYSQECGSFLLAESIKPAPPFNVTVTFSGQYNISWRSDYEDPAFY |
| | MLKGKLQYELQYRNRGDPWAVSPRRKLISVDSRSVSLLPLEFRKD |
| | SSYELQVRAGPMPGSSYQGTWSEWSDPVIFQTQSEELKE |
| 240 | QVQLQQWGAGLLKPSETLSLTCAVYGGSFSDYYWNWIRQPPGKG |
| D72A | LEWIGEINHRGSTNSNPSLKSRVTLSLDTSKNQFSLKLRSVTAADT |
| (αLAG-3 + | AVYYCAFGYSDYEYNWFDPWGQGTLVTVSSASTKGPSVFPLAPCS |
| linker + IL21Rα | RSTSESTAALGCLVKDYFPEPVTVSWNSGALTSGVHTFPAVLQSSG |
| Mutein) | LYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGK |
| | EYKCKVSNKGLPSSIEKTISKAKGQPREPQVYTLPPSQEEMTKNQV |
| | SLSCAVKGFYPSDIAVEWESNGQPENNYKTTPPVLDSDGSFFLVSR |
| | LTVDKSRWQEGNVFSCSVMHEALHNHYTQKSLSLSLGGGGGSGG |
| | GGSGGGGSCPDLVCYTDYLQTVICILEMWNLHPSTLTLTWQDQYE |
| | ELKDEATSCSLHRSAHNATHATYTCHMDVFHFMAADIFSVNITDQ |
| | SGNYSQECGSFLLAESIKPAPPFNVTVTFSGQYNISWRSDYEDPAFY |
| | MLKGKLQYELQYRNRGDPWAVSPRRKLISVDSRSVSLLPLEFRKD |
| | SSYELQVRAGPMPGSSYQGTWSEWSDPVIFQTQSEELKE |

1. An immunocytokine, comprising:
A. an antigen binding protein (ABP) specific to a target protein, optionally wherein the target protein is an immune checkpoint molecule;
B. an IL-21 domain; and
C. an IL-21Rα mutein,
wherein the IL-21Rα mutein has a reduced binding affinity to the IL-21 domain compared to a wild-type IL-21Rα.

2. (canceled)

3. The immunocytokine of claim 1, wherein the target protein is PD-1, PD-L1, TIGIT, LAG-3, CTLA-4, TIM-3, CD39, CD38, CD73, CD36, CD25, CD47, CD24, CD20, SIPRα, CD40, or CD20.

4. (canceled)

5. The immunocytokine of claim 1, wherein the ABP comprises Fc fragment, optionally selected from a human IgG1 Fc fragment, a human IgG2 Fc fragment, a human IgG3 Fc fragment, or a human IgG4 Fc fragment.

6. (canceled)

7. The immunocytokine of claim 1, wherein the ABP comprises Fc fragment, wherein the Fc fragment comprises a sequence selected from SEQ ID NOs: 16, 185-190.

8. The immunocytokine of claim 1, wherein the ABP comprises an Fc fragment comprising two Fc moieties, and the IL-21Rα mutein is linked to the first of the two Fc moieties, and the IL-21 domain is linked to the second of the two Fc moieties.

9. The immunocytokine of claim 8, wherein the IL-21 domain and the IL-21Rα mutein are respectively linked through a non-cleavable peptide linker or without a peptide linker.

10. (canceled)

11. The immunocytokine of claim 8, wherein the IL-21 domain and the IL-21Rα mutein are respectively linked through a non-cleavable peptide linker, wherein the non-cleavable peptide linker is G4S linker having the sequence of SEQ ID NO: 17 or a peptide linker having a sequence selected from SEQ ID NOs: 212-224.

12. (canceled)

13. The immunocytokine of claim 1, wherein the ABP comprises VH CDR1, VH CDR2, VH CDR3, VL CDR1, VL CDR2, and VL CDR3 sequences identical to an antibody selected from nivolumab, pembrolizumab, cemiplimab, atezolizumab, dostarlimab, durvalumab, avelumab, ipilimumab, tiragolumab, relatlimab, and tremelimumab.

14. The immunocytokine of claim 1, wherein the IL-21Rα mutein has 10 to 10,000-fold decrease in binding affinity to the IL-21 domain compared to a wild-type IL-21Rα.

15. The immunocytokine of claim 1, wherein the IL-21Rα mutein has a sequence with at least 95%, 96%, 97%, 98%, or 99% sequence identity to SEQ ID NO: 15.

16. (canceled)

17. The immunocytokine of claim 1, wherein the IL-21Rα mutein comprises one to five amino acid substitutions compared to SEQ ID NO: 15, optionally wherein the one or more amino acid substitutions are at one or more amino acid positions selected from Y10, Q35, Y36, E38, L39, F67, H68, M70, A71, D72, D73, I74, L94, P126, Y129, M130, K134, S189, S190, and Y191 in SEQ ID NO: 15.

18-19. (canceled)

20. The immunocytokine of claim 1, wherein the IL-21Rα mutein comprises a sequence selected from SEQ ID NOs: 18-99 and 155-169.

21. The immunocytokine of claim 1, comprising a first chain comprising from the N terminus to C terminus:
A. a first Fc moiety or a first heavy chain of a human IgG1, IgG2, IgG3 or IgG4, wherein the first Fc moiety or the first heavy chain comprises a knob-and-hole mutation; and
B. the IL-21Rα mutein.

22. The immunocytokine of claim 21,
wherein the IL-21Rα mutein comprises one or more amino acid mutations at one or more amino acid positions selected from Y10, Q35, Y36, E38, L39, F67, H68, M70, A71, D72, D73, I74, L94, P126, Y129, M130, K134, S189, S190, and Y191 in SEQ ID NO: 15.

23. (canceled)

24. The immunocytokine of claim 22, wherein the first chain comprises a sequence selected from SEQ ID NOs: 104-150, 192-209, 231-232, 235-236, and 239-240.

25-26. (canceled)

27. The immunocytokine of claim 1, comprising a second chain comprising a heavy chain of the ABP and the IL-21 domain, optionally wherein the second chain has the sequence of SEQ ID NO: 101, 229, 233 or 237.

28. (canceled)

29. The immunocytokine of claim 1, comprising a light chain having the sequence of SEQ ID NO: 102, 152, 226 or 228.

30. One or more polynucleotides encoding the immunocytokine of claim 1.

31-35. (canceled)

36. A host cell comprising the one or more polynucleotides of claim 30.

37-38. (canceled)

39. A method of enhancing immune response or a method of treating cancer in a subject, comprising administration of the immunocytokine of claim 1 to the subject.

40-42. (canceled)

43. An IL-21Rα mutein having a reduced binding affinity to an IL-21 domain compared to a wild-type IL-21Rα.

44-51. (canceled)


SEQUENCE LISTING (240 sequences)

SEQ ID NO: 1 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 15 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 16 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 15 aa, synthetic construct
GGGGSGGGGSGGGGS

SEQ ID NO: 18 - protein, 213 aa, synthetic construct
CPDLVCYTDYLQTVICILEMWNLHPSTLTLTWQDKYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 19 - protein, 213 aa, synthetic construct
CPDLVCYTDYLQTVICILEMWNLHPSTLTLTWQDRYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 20 - protein, 213 aa, synthetic construct
CPDLVCYTDYLQTVICILEMWNLHPSTLTLTWQDYYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 21 - protein, 213 aa, synthetic construct
CPDLVCYTDYLQTVICILEMWNLHPSTLTLTWQDQCEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 22 - protein, 213 aa, synthetic construct
CPDLVCYTDYLQTVICILEMWNLHPSTLTLTWQDQEEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 23 - protein, 213 aa, synthetic construct
CPDLVCYTDYLQTVICILEMWNLHPSTLTLTWQDQGEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 24 - protein, 213 aa, synthetic construct
CPDLVCYTDYLQTVICILEMWNLHPSTLTLTWQDQHEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 25 - protein, 213 aa, synthetic construct
CPDLVCYTDYLQTVICILEMWNLHPSTLTLTWQDQIEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 26 - protein, 213 aa, synthetic construct
CPDLVCYTDYLQTVICILEMWNLHPSTLTLTWQDQKEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 27 - protein, 213 aa, synthetic construct
CPDLVCYTDYLQTVICILEMWNLHPSTLTLTWQDQMEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 28 - protein, 213 aa, synthetic construct
CPDLVCYTDYLQTVICILEMWNLHPSTLTLTWQDQNEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 29 - protein, 213 aa, synthetic construct
CPDLVCYTDYLQTVICILEMWNLHPSTLTLTWQDQPEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 30 - protein, 213 aa, synthetic construct
CPDLVCYTDYLQTVICILEMWNLHPSTLTLTWQDQQEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 31 - protein, 213 aa, synthetic construct
CPDLVCYTDYLQTVICILEMWNLHPSTLTLTWQDQREELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 32 - protein, 213 aa, synthetic construct
CPDLVCYTDYLQTVICILEMWNLHPSTLTLTWQDQSEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 33 - protein, 213 aa, synthetic construct
CPDLVCYTDYLQTVICILEMWNLHPSTLTLTWQDQTEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 34 - protein, 213 aa, synthetic construct
CPDLVCYTDYLQTVICILEMWNLHPSTLTLTWQDQVEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 35 - protein, 213 aa, synthetic construct
CPDLVCYTDYLQTVICILEMWNLHPSTLTLTWQDQYECLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 36 - protein, 213 aa, synthetic construct
CPDLVCYTDYLQTVICILEMWNLHPSTLTLTWQDQYEKLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 37 - protein, 213 aa, synthetic construct
CPDLVCYTDYLQTVICILEMWNLHPSTLTLTWQDQYERLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 38 - protein, 213 aa, synthetic construct
CPDLVCYTDYLQTVICILEMWNLHPSTLTLTWQDQYEYLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 39 - protein, 213 aa, synthetic construct
CPDLVCYTDYLQTVICILEMWNLHPSTLTLTWQDQYEECKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 40 - protein, 213 aa, synthetic construct
CPDLVCYTDYLQTVICILEMWNLHPSTLTLTWQDQYEEEKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 41 - protein, 213 aa, synthetic construct
CPDLVCYTDYLQTVICILEMWNLHPSTLTLTWQDQYEEFKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 42 - protein, 213 aa, synthetic construct
CPDLVCYTDYLQTVICILEMWNLHPSTLTLTWQDQYEEHKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 43 - protein, 213 aa, synthetic construct
CPDLVCYTDYLQTVICILEMWNLHPSTLTLTWQDQYEEKKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 44 - protein, 213 aa, synthetic construct
CPDLVCYTDYLQTVICILEMWNLHPSTLTLTWQDQYEERKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 45 - protein, 213 aa, synthetic construct
CPDLVCYTDYLQTVICILEMWNLHPSTLTLTWQDQYEEWKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 46 - protein, 213 aa, synthetic construct
CPDLVCYTDYLQTVICILEMWNLHPSTLTLTWQDQYEEYKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 47 - protein, 213 aa, synthetic construct
CPDLVCYTDYLQTVICILEMWNLHPSTLTLTWQDQYEELKDEATSCSLHRSAHNATHATYTCHMDVFHFCADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 48 - protein, 213 aa, synthetic construct
CPDLVCYTDYLQTVICILEMWNLHPSTLTLTWQDQYEELKDEATSCSLHRSAHNATHATYTCHMDVFHFDADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 49 - protein, 213 aa, synthetic construct
CPDLVCYTDYLQTVICILEMWNLHPSTLTLTWQDQYEELKDEATSCSLHRSAHNATHATYTCHMDVFHFFADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 50 - protein, 213 aa, synthetic construct
CPDLVCYTDYLQTVICILEMWNLHPSTLTLTWQDQYEELKDEATSCSLHRSAHNATHATYTCHMDVFHFGADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 51 - protein, 213 aa, synthetic construct
CPDLVCYTDYLQTVICILEMWNLHPSTLTLTWQDQYEELKDEATSCSLHRSAHNATHATYTCHMDVFHFHADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 52 - protein, 213 aa, synthetic construct
CPDLVCYTDYLQTVICILEMWNLHPSTLTLTWQDQYEELKDEATSCSLHRSAHNATHATYTCHMDVFHFKADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 53 - protein, 213 aa, synthetic construct
CPDLVCYTDYLQTVICILEMWNLHPSTLTLTWQDQYEELKDEATSCSLHRSAHNATHATYTCHMDVFHFLADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 54 - protein, 213 aa, synthetic construct
CPDLVCYTDYLQTVICILEMWNLHPSTLTLTWQDQYEELKDEATSCSLHRSAHNATHATYTCHMDVFHFNADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 55 - protein, 213 aa, synthetic construct
CPDLVCYTDYLQTVICILEMWNLHPSTLTLTWQDQYEELKDEATSCSLHRSAHNATHATYTCHMDVFHFQADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 56 - protein, 213 aa, synthetic construct
CPDLVCYTDYLQTVICILEMWNLHPSTLTLTWQDQYEELKDEATSCSLHRSAHNATHATYTCHMDVFHFRADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 57 - protein, 213 aa, synthetic construct
CPDLVCYTDYLQTVICILEMWNLHPSTLTLTWQDQYEELKDEATSCSLHRSAHNATHATYTCHMDVFHFSADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 58 - protein, 213 aa, synthetic construct
CPDLVCYTDYLQTVICILEMWNLHPSTLTLTWQDQYEELKDEATSCSLHRSAHNATHATYTCHMDVFHFTADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 59 - protein, 213 aa, synthetic construct
CPDLVCYTDYLQTVICILEMWNLHPSTLTLTWQDQYEELKDEATSCSLHRSAHNATHATYTCHMDVFHFVADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 60 - protein, 213 aa, synthetic construct
CPDLVCYTDYLQTVICILEMWNLHPSTLTLTWQDQYEELKDEATSCSLHRSAHNATHATYTCHMDVFHFWADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 61 - protein, 213 aa, synthetic construct
CPDLVCYTDYLQTVICILEMWNLHPSTLTLTWQDQYEELKDEATSCSLHRSAHNATHATYTCHMDVFHFYADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 62 - protein, 213 aa, synthetic construct
CPDLVCYTDYLQTVICILEMWNLHPSTLTLTWQDQYEELKDEATSCSLHRSAHNATHATYTCHMDVFHFMEDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 63 - protein, 213 aa, synthetic construct
CPDLVCYTDYLQTVICILEMWNLHPSTLTLTWQDQYEELKDEATSCSLHRSAHNATHATYTCHMDVFHFMFDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 64 - protein, 213 aa, synthetic construct
CPDLVCYTDYLQTVICILEMWNLHPSTLTLTWQDQYEELKDEATSCSLHRSAHNATHATYTCHMDVFHFMIDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 65 - protein, 213 aa, synthetic construct
CPDLVCYTDYLQTVICILEMWNLHPSTLTLTWQDQYEELKDEATSCSLHRSAHNATHATYTCHMDVFHFMLDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 66 - protein, 213 aa, synthetic construct
CPDLVCYTDYLQTVICILEMWNLHPSTLTLTWQDQYEELKDEATSCSLHRSAHNATHATYTCHMDVFHFMQDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 67 - protein, 213 aa, synthetic construct
CPDLVCYTDYLQTVICILEMWNLHPSTLTLTWQDQYEELKDEATSCSLHRSAHNATHATYTCHMDVFHFMRDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 68 - protein, 213 aa, synthetic construct
CPDLVCYTDYLQTVICILEMWNLHPSTLTLTWQDQYEELKDEATSCSLHRSAHNATHATYTCHMDVFHFMWDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 69 - protein, 213 aa, synthetic construct
CPDLVCYTDYLQTVICILEMWNLHPSTLTLTWQDQYEELKDEATSCSLHRSAHNATHATYTCHMDVFHFMYDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 70 - protein, 213 aa, synthetic construct
CPDLVCYTDYLQTVICILEMWNLHPSTLTLTWQDQYEELKDEATSCSLHRSAHNATHATYTCHMDVFHFMAADIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 71 - protein, 213 aa, synthetic construct
CPDLVCYTDYLQTVICILEMWNLHPSTLTLTWQDQYEELKDEATSCSLHRSAHNATHATYTCHMDVFHFMACDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 72 - protein, 213 aa, synthetic construct
CPDLVCYTDYLQTVICILEMWNLHPSTLTLTWQDQYEELKDEATSCSLHRSAHNATHATYTCHMDVFHFMAEDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 73 - protein, 213 aa, synthetic construct
CPDLVCYTDYLQTVICILEMWNLHPSTLTLTWQDQYEELKDEATSCSLHRSAHNATHATYTCHMDVFHFMAFDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 74 - protein, 213 aa, synthetic construct
CPDLVCYTDYLQTVICILEMWNLHPSTLTLTWQDQYEELKDEATSCSLHRSAHNATHATYTCHMDVFHFMAGDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 75 - protein, 213 aa, synthetic construct
CPDLVCYTDYLQTVICILEMWNLHPSTLTLTWQDQYEELKDEATSCSLHRSAHNATHATYTCHMDVFHFMAHDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 76 - protein, 213 aa, synthetic construct
CPDLVCYTDYLQTVICILEMWNLHPSTLTLTWQDQYEELKDEATSCSLHRSAHNATHATYTCHMDVFHFMAIDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 77 - protein, 213 aa, synthetic construct
CPDLVCYTDYLQTVICILEMWNLHPSTLTLTWQDQYEELKDEATSCSLHRSAHNATHATYTCHMDVFHFMAKDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 78 - protein, 213 aa, synthetic construct
CPDLVCYTDYLQTVICILEMWNLHPSTLTLTWQDQYEELKDEATSCSLHRSAHNATHATYTCHMDVFHFMALDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 79 - protein, 213 aa, synthetic construct
CPDLVCYTDYLQTVICILEMWNLHPSTLTLTWQDQYEELKDEATSCSLHRSAHNATHATYTCHMDVFHFMAMDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 80 - protein, 213 aa, synthetic construct
CPDLVCYTDYLQTVICILEMWNLHPSTLTLTWQDQYEELKDEATSCSLHRSAHNATHATYTCHMDVFHFMAQDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 81 - protein, 213 aa, synthetic construct
CPDLVCYTDYLQTVICILEMWNLHPSTLTLTWQDQYEELKDEATSCSLHRSAHNATHATYTCHMDVFHFMARDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 82 - protein, 213 aa, synthetic construct
CPDLVCYTDYLQTVICILEMWNLHPSTLTLTWQDQYEELKDEATSCSLHRSAHNATHATYTCHMDVFHFMAWDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 83 - protein, 213 aa, synthetic construct
CPDLVCYTDYLQTVICILEMWNLHPSTLTLTWQDQYEELKDEATSCSLHRSAHNATHATYTCHMDVFHFMAYDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 84 - protein, 213 aa, synthetic construct
CPDLVCYTDYLQTVICILEMWNLHPSTLTLTWQDQYEELKDEATSCSLHRSAHNATHATYTCHMDVFHFMADCIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 85 - protein, 213 aa, synthetic construct
CPDLVCYTDYLQTVICILEMWNLHPSTLTLTWQDQYEELKDEATSCSLHRSAHNATHATYTCHMDVFHFMADAIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 86 - protein, 213 aa, synthetic construct
CPDLVCYTDYLQTVICILEMWNLHPSTLTLTWQDQYEELKDEATSCSLHRSAHNATHATYTCHMDVFHFMADEIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 87 - protein, 213 aa, synthetic construct
CPDLVCYTDYLQTVICILEMWNLHPSTLTLTWQDQYEELKDEATSCSLHRSAHNATHATYTCHMDVFHFMADHIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 88 - protein, 213 aa, synthetic construct
CPDLVCYTDYLQTVICILEMWNLHPSTLTLTWQDQYEELKDEATSCSLHRSAHNATHATYTCHMDVFHFMADKIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 89 - protein, 213 aa, synthetic construct
CPDLVCYTDYLQTVICILEMWNLHPSTLTLTWQDQYEELKDEATSCSLHRSAHNATHATYTCHMDVFHFMADRIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 90 - protein, 213 aa, synthetic construct
CPDLVCYTDYLQTVICILEMWNLHPSTLTLTWQDQYEELKDEATSCSLHRSAHNATHATYTCHMDVFHFMADWIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 91 - protein, 213 aa, synthetic construct
CPDLVCYTDYLQTVICILEMWNLHPSTLTLTWQDQYEELKDEATSCSLHRSAHNATHATYTCHMDVFHFMADYIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 92 - protein, 213 aa, synthetic construct
CPDLVCYTDYLQTVICILEMWNLHPSTLTLTWQDQYEELKDEATSCSLHRSAHNATHATYTCHMDVFHFMADDHFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 93 - protein, 213 aa, synthetic construct
CPDLVCYTDYLQTVICILEMWNLHPSTLTLTWQDQYEELKDEATSCSLHRSAHNATHATYTCHMDVFHFMADDKFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 94 - protein, 213 aa, synthetic construct
CPDLVCYTDYLQTVICILEMWNLHPSTLTLTWQDQYEELKDEATSCSLHRSAHNATHATYTCHMDVFHFMADDRFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 95 - protein, 213 aa, synthetic construct
CPDLVCYTDYLQTVICILEMWNLHPSTLTLTWQDQYEELKDEATSCSLHRSAHNATHATYTCHMDVFHFMADDWFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 96 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFFLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 97 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFKLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 98 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFQLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 99 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFRLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 100 - protein, 131 aa, Homo sapiens
QDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 101 - protein, 585 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 102 - protein, 214 aa, synthetic construct; identical to SEQ ID NO: 2

SEQ ID NO: 103 - protein, 439 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 104 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQCEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 105 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQEEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 106 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQGEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 107 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQHEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 108 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQIEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 109 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQKEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 110 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQMEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 111 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQNEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 112 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQPEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 113 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQQEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 114 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQREELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 115 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQSEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 116 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQTEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 117 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQVEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 118 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYECLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 119 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEYLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 120 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEECKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 121 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEEEKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 122 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEEHKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 123 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEEKKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 124 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEERKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 125 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEEWKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 126 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEEYKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 127 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFFADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 128 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFHADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 129 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFNADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 130 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFQADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 131 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFSADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 132 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFTADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 133 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFVADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 134 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFWADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 135 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFYADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 136 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMEDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 137 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMFDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 138 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMIDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 139 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMLDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 140 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMQDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 141 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMRDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 142 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMWDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 143 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMYDDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 144 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDRFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 145 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDWFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 146 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFFLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 147 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFKLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 148 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFQLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 149 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFRLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 150 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFYLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 151 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 154 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 213 aa, synthetic construct
CPDLVCYTDALQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 156 - protein, 213 aa, synthetic construct
CPDLVCYTDYLQTVICILEMWNLHPSTLTLTWQDQAEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 157 - protein, 213 aa, synthetic construct
CPDLVCYTDYLQTVICILEMWNLHPSTLTLTWQDQYEALKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 158 - protein, 213 aa, synthetic construct
CPDLVCYTDYLQTVICILEMWNLHPSTLTLTWQDQYEEAKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 159 - protein, 213 aa, synthetic construct
CPDLVCYTDYLQTVICILEMWNLHPSTLTLTWQDQYEELKDEATSCSLHRSAHNATHATYTCHMDVAHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 160 - protein, 213 aa, synthetic construct
CPDLVCYTDYLQTVICILEMWNLHPSTLTLTWQDQYEELKDEATSCSLHRSAHNATHATYTCHMDVFAFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 161 - protein, 213 aa, synthetic construct
CPDLVCYTDYLQTVICILEMWNLHPSTLTLTWQDQYEELKDEATSCSLHRSAHNATHATYTCHMDVFHFMADDAFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 162 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFALAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 163 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDAAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 164 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFAMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 165 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYALKGKLQYELQYRNRGDPWAVSPRRKLISVDSRSVSLLPLEFRKDSSYELQVRAGPMPGSSYQGTWSEWSDPVIFQTQSEELKE

SEQ ID NO: 166 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGALQYELQYRNRGDPWAVSPRRKLISVDSRSVSLLPLEFRKDSSYELQVRAGPMPGSSYQGTWSEWSDPVIFQTQSEELKE

SEQ ID NO: 167 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGASYQGTWSEWSDPVIFQTQSEELKE

SEQ ID NO: 168 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSAYQGTWSEWSDPVIFQTQSEELKE

SEQ ID NO: 169 - protein, 213 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSAQGTWSEWSDPVIFQTQSEELKE

SEQ ID NO: 170 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDALQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 171 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQAEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 172 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEALKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 173 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEEAKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 174 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVAHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 175 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFAFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 176 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDAFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 177 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFALAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 178 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDAAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 179 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFAMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 180 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYALKGKLQYELQYRNRGDPWAVSPRRKLISVDSRSVSLLPLEFRKDSSYELQVRAGPMPGSSYQGTWSEWSDPVIFQTQSEELKE

SEQ ID NO: 181 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGALQYELQYRNRGDPWAVSPRRKLISVDSRSVSLLPLEFRKDSSYELQVRAGPMPGSSYQGTWSEWSDPVIFQTQSEELKE

SEQ ID NO: 182 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGASYQGTWSEWSDPVIFQTQSEELKE

SEQ ID NO: 183 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSAYQGTWSEWSDPVIFQTQSEELKE

SEQ ID NO: 184 - protein, 459 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSAQGTWSEWSDPVIFQTQSEELKE

SEQ ID NO: 185 - protein, 231 aa, Homo sapiens; identical to SEQ ID NO: 16

SEQ ID NO: 186 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 187 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 188 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 189 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 191 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 192 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQAEELKDEATSCSLHRSAHNATHATYTCHMDVFHFMAEDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 193 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQAEELKDEATSCSLHRSAHNATHATYTCHMDVFHFMADDIFSVNITDQSGNYSQECGSFRLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 194 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEALKDEATSCSLHRSAHNATHATYTCHMDVFHFMAEDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 195 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEALKDEATSCSLHRSAHNATHATYTCHMDVFHFMADDIFSVNITDQSGNYSQECGSFKLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 196 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEALKDEATSCSLHRSAHNATHATYTCHMDVFHFMADDIFSVNITDQSGNYSQECGSFRLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 197 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYERLKDEATSCSLHRSAHNATHATYTCHMDVFHFMARDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 198 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMAEDIFSVNITDQSGNYSQECGSFKLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 199 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMAEDIFSVNITDQSGNYSQECGSFRLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 200 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYERLKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 201 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFCADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 202 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFDADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 203 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFGADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 204 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFRADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 205 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMAADIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 206 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMAEDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 207 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMAQDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 208 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMARDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 209 - protein, 667 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADAIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 210 - DNA, 1377 bp, synthetic construct
  source 1..1377: mol_type = other DNA
ccgaaatcatgtgacaaaactcatacttgtcctccatgcccagccccagaattgctggggggaccatctgtgttccttttcccccctaagccaaaagacactctgatgatcagtcgcactcctgaagtgacctgcgtcgtggtagacgtctctcacgaagatcccgaggtcaaatttaactggtatgtggatggcgtggaagttcataacgcaaaaaccaaaccccgcgaagaacaatataatagcacataccgtgttgttagcgttttgacagtccttcaccaggattggctcaacggaaaagagtacaagtgcaaggtgtccaataaagcattgcccgcccctatagagaagactattagcaaggccaaaggtcagccccgggagcctcaggtgtatacattgcctcccagccgcgatgaactcactaaaaaccaagtcagcctcacatgtctggttaaaggtttttaccccagcgatatcgcagtcgagtgggaatctaatgggcagcctgaaaataactataagacaaccccaccagtgttggatagcgatggcagcttttttctttactctaagttgactgttgacaagagcaggtggcaacaaggcaacgtgtttagctgcagtgtcatgcacgaagcactccacaatcattacacccagaagagtctgagcttgtcacctggaaagggtggaggcggttcaggcggaggtggctctggcggtggcggatcatgtcctgacctggtgtgctacaccgactacctgcagaccgtgatctgcatcctggagatgtggaacctgcatccttctaccctgacactgacctggcaggaccagtacgaggaactgaaggacgaggccacctcctgctccctgcacagatctgctcacaacgccacccacgctacctacacctgtcacatggacgtgttccacttcatggccgacgacatcttttctgtgaacatcaccgatcagtctggcaactactcccaagagtgcggctctttcctgctggccgagtccatcaagcctgctcctcctttcaacgtgaccgtgaccttctccggccagtacaacatctcttggcggtccgactacgaggaccccgccttctacatgctgaagggcaagctgcagtacgagctgcagtaccggaacagaggcgacccttgggccgtgtcccctagaagaaagctgatctccgtggactccagatccgtgtctctgctgcctctggaattccggaaggactctagctacgaactgcaagtgcgggctggccctatgcctggctcctcctaccagggaacatggtccgagtggagcgatcctgtgatcttccagacccagtccgaagagctgaaagag

SEQ ID NO: 211 - DNA, 2001 bp, synthetic construct
  source 1..2001: mol_type = other DNA
caggtgcagctggtggagtccggaggaggagtggtgcagccaggcaggtccctgcggctggactgtaaggcctccggcatcaccttttctaactccggaatgcattgggtgaggcaggctccaggcaagggcctggagtgggtggctgtgatctggtacgacggcagcaagcggtactatgccgattctgtgaagggcagattcacaatctctcgcgacaactccaagaataccctgtttctgcagatgaactctctgagggccgaggatacagccgtgtactattgcgctaccaatgacgattactggggccagggcacactggtgaccgtgtccagcgccagcacaaagggaccatccgtgttcccactggctccatgcagccggtctacatccgagagcaccgccgctctgggatgtctggtgaaggattatttccctgagccagtgaccgtgagctggaactccggcgccctgacatctggcgtgcacacctttcctgctgtgctgcagtcttccggcctgtactccctgagctctgtggtgacagtgccctccagctctctgggcaccaagacatatacctgcaacgtggaccataagccttccaataccaaggtggataagagagtggagagcaagtacggaccaccttgcccaccatgtccagctcctgagtttctgggaggaccatccgtgttcctgtttcctccaaagcctaaggacaccctgatgatcagccgcacacctgaggtgacctgcgtggtggtggacgtgtctcaggaggaccccgaggtgcagttcaactggtacgtggatggcgtggaggtgcacaatgctaagaccaagcctagagaggagcagtttaactccacataccgcgtggtgagcgtgctgaccgtgctgcatcaggactggctgaacggcaaggagtataagtgcaaggtgtccaataagggcctgccatccagcatcgagaagacaatcagcaaggccaagggccagcctagggagccacaggtgtacaccctgcccccttctcaggaggagatgacaaagaaccaggtgtccctgtcctgtgccgtgaagggcttctatccaagcgacatcgctgtggagtgggagtctaatggccagcccgagaacaattacaagaccacaccacccgtgctggactccgatggcagcttctttctggtctccaggctgacagtggataagagccggtggcaggagggcaacgtgttttcttgttccgtgatgcacgaggctctgcacaatcattacacccagaagagcctgtctctgtccctgggcggtggcggtggctctggcggaggtggctcaggtggcggcggatcctgtcctgatctcgtgtgctataccgactacctccagaccgttatttgtatccttgagatgtggaatttgcacccatcaacactgactctgacttggcaggatcaatacgaggagctgaaagacgaggccacatcctgctccttgcatcgatcagcacacaacgccactcatgcaacatacacttgccatatggatgtgttccacttcatggcagatgatattttttcagttaacattacagatcaatccggcaactattcacaggaatgtggctcttttcttctggcagaatcaataaagcccgcacctcctttcaacgtgactgtcaccttctcaggacaatataatatcagctggcgatctgactatgaggaccctgccttttacatgctgaaaggcaagctccaatacgaacttcaatatcgtaataggggggacccatgggccgtcagtcctcgacggaagctgatatccgtggactctagaagtgtctctctcttgcccctcgaatttaggaaagactcatcctacgagcttcaagttcgggcaggtcccatgcccggctcaagctatcaggggacatggagcgagtggtccgacccagtaattttccaaacccaaagcgaggaattgaaagag

SEQ ID NO: 212 - protein, 5 aa, synthetic construct
GGGGS

SEQ ID NO: 213 - protein, 10 aa, synthetic construct
GGGGSGGGGS

SEQ ID NO: 214 - protein, 15 aa, synthetic construct
GGGGSGGGGSGGGGS

SEQ ID NO: 215 - protein, 20 aa, synthetic construct
GGGGSGGGGSGGGGSGGGGS

SEQ ID NO: 216 - protein, 6 aa, synthetic construct
GGGGGG

SEQ ID NO: 217 - protein, 8 aa, synthetic construct
GGGGGGGG

SEQ ID NO: 218 - protein, 5 aa, synthetic construct
EAAAK

SEQ ID NO: 219 - protein, 10 aa, synthetic construct
EAAAKEAAAK

SEQ ID NO: 220 - protein, 15 aa, synthetic construct
EAAAKEAAAKEAAAK

SEQ ID NO: 221 - protein, 46 aa, synthetic construct
AEAAAKEAAAKEAAAKEAAAKALEAEAAAKEAAAKEAAAKEAAAKA

SEQ ID NO: 222 - protein, 5 aa, synthetic construct
PAPAP

SEQ ID NO: 223 - protein, 12 aa, synthetic construct
AEAAAKEAAAKA

SEQ ID NO: 224 - protein, 30 aa, synthetic construct
  VARIANT 1..30: note = This sequence may encompass 5-15 AP repeating units
APAPAPAPAPAPAPAPAPAPAPAPAPAPAP

SEQ ID NO: 225 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 447 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 214 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 593 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 230 - protein, 675 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 231 - protein, 675 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSCPDLVCYTDYLQTVICILEMWNLHPSTLTLTWQDQYEELKDEATSCSLHRSAHNATHATYTCHMDVFHFDADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 232 - protein, 675 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSCPDLVCYTDYLQTVICILEMWNLHPSTLTLTWQDQYEELKDEATSCSLHRSAHNATHATYTCHMDVFHFMAADIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 233 - protein, 601 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 234 - protein, 683 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 235 - protein, 683 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSCPDLVCYTDYLQTVICILEMWNLHPSTLTLTWQDQYEELKDEATSCSLHRSAHNATHATYTCHMDVFHFDADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 236 - protein, 683 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSCPDLVCYTDYLQTVICILEMWNLHPSTLTLTWQDQYEELKDEATSCSLHRSAHNATHATYTCHMDVFHFMAADIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 237 - protein, 592 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 238 - protein, 674 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 239 - protein, 674 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFDADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 240 - protein, 674 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSCPDLVCYTDYLQTVICILEMWNLHPSTLTLTWQDQYEELKDEATSCSLHRSAHNATHATYTCHMDVFHFMAADIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE